(19) United States
(12) Patent Application Publication
(10) Pub. No.: US 2026/0295189 A1
(43) Pub. Date: Oct. 1, 2026

(54) PATIENT INTERFACE AND CUSHION THEREOF

(71) Applicant: RESMED PTY LTD, Bella Vista, New South Wales (AU)

(72) Inventors: Andrew James BATE, Sydney (AU); Vanessa GRAY, Sydney (AU); Michael Christopher HOGG, Sydney (AU); Stewart Joseph WAGNER, Hawkesbury (AU); Jie YUAN, Sydney (AU); Aaron Samuel DAVIDSON, Sydney (AU); Ian Andrew LAW, Sydney (AU)

(21) Appl. No.: 18/992,836

(22) PCT Filed: Jul. 14, 2023
(86) PCT No.: PCT/AU2023/050649

§ 371 Date: Jan. 9, 2025

(30) Foreign Application Priority Data
Jul. 14, 2022 (AU) 2022901964

(51) Int. Cl. A61M 16/06

(52) U.S. Cl. CPC A61M 16/0622; A61M 2016/0661; A61M 2205/02

(57) ABSTRACT

A patient interface comprising a plenum chamber, a seal-forming structure constructed and arranged to form a seal with a region of the patient's face surrounding an entrance to the patient's airways, and a vent to allow a continuous flow of gases exhaled by the patient from an interior of the plenum chamber to ambient. The seal-forming structure may comprise a cushion, the cushion being deformable and resilient and at least partially formed by a lattice structure.

### 1 CROSS-REFERENCE TO RELATED APPLICATIONS

This application claims the benefit of Australian Patent Application No. 2022901964 filed 14 Jul. 2022, the entire contents of which are hereby incorporated by reference herein.

### 2 BACKGROUND OF THE TECHNOLOGY

### 2.1 Field of the Technology

The present technology relates to one or more of the screening, diagnosis, monitoring, treatment, prevention and amelioration of respiratory-related disorders.

The present technology also relates to medical devices or apparatus, and their use.

### 2.2 Description of the Related Art

### 2.2.1 Human Respiratory System and its Disorders

The respiratory system of the body facilitates gas exchange. The nose and mouth form the entrance to the airways of a patient.

The airways include a series of branching tubes, which become narrower, shorter and more numerous as they penetrate deeper into the lung. The prime function of the lung is gas exchange, allowing oxygen to move from the inhaled air into the venous blood and carbon dioxide to move in the opposite direction. The trachea divides into right and left main bronchi, which further divide eventually into terminal bronchioles. The bronchi make up the conducting airways, and do not take part in gas exchange. Further divisions of the airways lead to the respiratory bronchioles, and eventually to the alveoli. The alveolated region of the lung is where the gas exchange takes place, and is referred to as the respiratory zone. See “Respiratory Physiology”, by John B. West, Lippincott Williams & Wilkins, 9th edition published 2012.

A range of respiratory disorders exist. Certain disorders may be characterised by particular events, e.g. apneas, hypopneas, and hyperpneas.

Examples of respiratory disorders include Obstructive Sleep Apnea (OSA), Cheyne-Stokes Respiration (CSR), respiratory insufficiency, Obesity Hyperventilation Syndrome (OHS), Chronic Obstructive Pulmonary Disease (COPD), Neuromuscular Disease (NMD) and Chest wall disorders.

Obstructive Sleep Apnea (OSA), a form of Sleep Disordered Breathing (SDB), is characterised by events including occlusion or obstruction of the upper air passage during sleep. It results from a combination of an abnormally small upper airway and the normal loss of muscle tone in the region of the tongue, soft palate and posterior oropharyngeal wall during sleep. The condition causes the affected patient to stop breathing for periods typically of 30 to 120 seconds in duration, sometimes 200 to 300 times per night. It often causes excessive daytime somnolence, and it may cause cardiovascular disease and brain damage. The syndrome is a common disorder, particularly in middle aged overweight males, although a person affected may have no awareness of the problem. See U.S. Pat. No. 4,944,310 (Sullivan).

Cheyne-Stokes Respiration (CSR) is another form of sleep disordered breathing. CSR is a disorder of a patient's respiratory controller in which there are rhythmic alternating periods of waxing and waning ventilation known as CSR cycles. CSR is characterised by repetitive de-oxygenation and re-oxygenation of the arterial blood. It is possible that CSR is harmful because of the repetitive hypoxia. In some patients CSR is associated with repetitive arousal from sleep, which causes severe sleep disruption, increased sympathetic activity, and increased afterload. See U.S. Pat. No. 6,532,959 (Berthon-Jones).

Respiratory failure is an umbrella term for respiratory disorders in which the lungs are unable to inspire sufficient oxygen or exhale sufficient CO2 to meet the patient's needs. Respiratory failure may encompass some or all of the following disorders.

A patient with respiratory insufficiency (a form of respiratory failure) may experience abnormal shortness of breath on exercise.

Obesity Hyperventilation Syndrome (OHS) is defined as the combination of severe obesity and awake chronic hypercapnia, in the absence of other known causes for hypoventilation. Symptoms include dyspnea, morning headache and excessive daytime sleepiness.

Chronic Obstructive Pulmonary Disease (COPD) encompasses any of a group of lower airway diseases that have certain characteristics in common. These include increased resistance to air movement, extended expiratory phase of respiration, and loss of the normal elasticity of the lung. Examples of COPD are emphysema and chronic bronchitis. COPD is caused by chronic tobacco smoking (primary risk factor), occupational exposures, air pollution and genetic factors. Symptoms include: dyspnea on exertion, chronic cough and sputum production.

Neuromuscular Disease (NMD) is a broad term that encompasses many diseases and ailments that impair the functioning of the muscles either directly via intrinsic muscle pathology, or indirectly via nerve pathology. Some NMD patients are characterised by progressive muscular impairment leading to loss of ambulation, being wheelchair-bound, swallowing difficulties, respiratory muscle weakness and, eventually, death from respiratory failure. Neuromuscular disorders can be divided into rapidly progressive and slowly progressive: (i) Rapidly progressive disorders: Characterised by muscle impairment that worsens over months and results in death within a few years (e.g. Amyotrophic lateral sclerosis (ALS) and Duchenne muscular dystrophy (DMD) in teenagers); (ii) Variable or slowly progressive disorders: Characterised by muscle impairment that worsens over years and only mildly reduces life expectancy (e.g. Limb girdle, Facioscapulohumeral and Myotonic muscular dystrophy). Symptoms of respiratory failure in NMD include: increasing generalised weakness, dysphagia, dyspnea on exertion and at rest, fatigue, sleepiness, morning headache, and difficulties with concentration and mood changes.

Chest wall disorders are a group of thoracic deformities that result in inefficient coupling between the respiratory muscles and the thoracic cage. The disorders are usually characterised by a restrictive defect and share the potential of long term hypercapnic respiratory failure. Scoliosis and/or kyphoscoliosis may cause severe respiratory failure. Symptoms of respiratory failure include: dyspnea on exertion, peripheral oedema, orthopnea, repeated chest infections, morning headaches, fatigue, poor sleep quality and loss of appetite.

A range of therapies have been used to treat or ameliorate such conditions. Furthermore, otherwise healthy individuals may take advantage of such therapies to prevent respiratory disorders from arising. However, these have a number of shortcomings.

### 2.2.2 Therapies

Various respiratory therapies, such as Continuous Positive Airway Pressure (CPAP) therapy, Non-invasive ventilation (NIV), Invasive ventilation (IV), and High Flow Therapy (HFT) have been used to treat one or more of the above respiratory disorders.

### 2.2.2.1 Respiratory Pressure Therapies

Respiratory pressure therapy is the application of a supply of air to an entrance to the airways at a controlled target pressure that is nominally positive with respect to atmosphere throughout the patient's breathing cycle (in contrast to negative pressure therapies such as the tank ventilator or cuirass).

Continuous Positive Airway Pressure (CPAP) therapy has been used to treat Obstructive Sleep Apnea (OSA). The mechanism of action is that continuous positive airway pressure acts as a pneumatic splint and may prevent upper airway occlusion, such as by pushing the soft palate and tongue forward and away from the posterior oropharyngeal wall. Treatment of OSA by CPAP therapy may be voluntary, and hence patients may elect not to comply with therapy if they find devices used to provide such therapy one or more of: uncomfortable, difficult to use, expensive and aesthetically unappealing.

### 2.2.3 Respiratory Therapy Systems

These respiratory therapies may be provided by a respiratory therapy system or device. Such systems and devices may also be used to screen, diagnose, or monitor a condition without treating it.

A respiratory therapy system may comprise a Respiratory Pressure Therapy Device (RPT device), an air circuit, a humidifier, a patient interface, an oxygen source, and data management.

Another form of therapy system is a mandibular repositioning device.

### 2.2.3.1 Patient Interface

A patient interface may be used to interface respiratory equipment to its wearer, for example by providing a flow of air to an entrance to the airways. The flow of air may be provided via a mask to the nose and/or mouth, a tube to the mouth or a tracheostomy tube to the trachea of a patient. Depending upon the therapy to be applied, the patient interface may form a seal, e.g., with a region of the patient's face, to facilitate the delivery of gas at a pressure at sufficient variance with ambient pressure to effect therapy, e.g., at a positive pressure of about 10 cmH₂O relative to ambient pressure. For other forms of therapy, such as the delivery of oxygen, the patient interface may not include a seal sufficient to facilitate delivery to the airways of a supply of gas at a positive pressure of about 10 cmH₂O. For flow therapies such as nasal HFT, the patient interface is configured to insufflate the nares but specifically to avoid a complete seal. One example of such a patient interface is a nasal cannula.

Certain other mask systems may be functionally unsuitable for the present field. For example, purely ornamental masks may be unable to maintain a suitable pressure. Mask systems used for underwater swimming or diving may be configured to guard against ingress of water from an external higher pressure, but not to maintain air internally at a higher pressure than ambient.

Certain masks may be clinically unfavourable for the present technology e.g. if they block airflow via the nose and only allow it via the mouth.

Certain masks may be uncomfortable or impractical for the present technology if they require a patient to insert a portion of a mask structure in their mouth to create and maintain a seal via their lips.

Certain masks may be impractical for use while sleeping, e.g. for sleeping while lying on one's side in bed with a head on a pillow.

The design of a patient interface presents a number of challenges. The face has a complex three-dimensional shape. The size and shape of noses and heads varies considerably between individuals. Since the head includes bone, cartilage and soft tissue, different regions of the face respond differently to mechanical forces. The jaw or mandible may move relative to other bones of the skull. The whole head may move during the course of a period of respiratory therapy.

As a consequence of these challenges, some masks suffer from being one or more of obtrusive, aesthetically undesirable, costly, poorly fitting, difficult to use, and uncomfortable especially when worn for long periods of time or when a patient is unfamiliar with a system. Wrongly sized masks can give rise to reduced compliance, reduced comfort and poorer patient outcomes. Masks designed solely for aviators, masks designed as part of personal protection equipment (e.g. filter masks), SCUBA masks, or for the administration of anaesthetics may be tolerable for their original application, but nevertheless such masks may be undesirably uncomfortable to be worn for extended periods of time, e.g., several hours. This discomfort may lead to a reduction in patient compliance with therapy. This is even more so if the mask is to be worn during sleep.

CPAP therapy is highly effective to treat certain respiratory disorders, provided patients comply with therapy. If a mask is uncomfortable, or difficult to use a patient may not comply with therapy. Since it is often recommended that a patient regularly wash their mask, if a mask is difficult to clean (e.g., difficult to assemble or disassemble), patients may not clean their mask and this may impact on patient compliance.

While a mask for other applications (e.g. aviators) may not be suitable for use in treating sleep disordered breathing, a mask designed for use in treating sleep disordered breathing may be suitable for other applications.

For these reasons, patient interfaces for delivery of CPAP during sleep form a distinct field.

### 2.2.3.1.1 Seal-Forming Structure

Patient interfaces may include a seal-forming structure. Since it is in direct contact with the patient's face, the shape and configuration of the seal-forming structure can have a direct impact the effectiveness and comfort of the patient interface.

A patient interface may be partly characterised according to the design intent of where the seal-forming structure is to engage with the face in use. In one form of patient interface, a seal-forming structure may comprise a first sub-portion to form a seal around the left naris and a second sub-portion to form a seal around the right naris. In one form of patient interface, a seal-forming structure may comprise a single element that surrounds both nares in use. Such single element may be designed to for example overlay an upper lip region and a nasal bridge region of a face. In one form of patient interface a seal-forming structure may comprise an element that surrounds a mouth region in use, e.g. by forming a seal on a lower lip region of a face. In one form of patient interface, a seal-forming structure may comprise a single element that surrounds both nares and a mouth region in use. These different types of patient interfaces may be known by a variety of names by their manufacturer including nasal masks, full-face masks, nasal pillows, nasal puffs and oro-nasal masks.

A seal-forming structure that may be effective in one region of a patient's face may be inappropriate in another region, e.g. because of the different shape, structure, variability and sensitivity regions of the patient's face. For example, a seal on swimming goggles that overlays a patient's forehead may not be appropriate to use on a patient's nose.

Certain seal-forming structures may be designed for mass manufacture such that one design is able to fit and be comfortable and effective for a wide range of different face shapes and sizes. To the extent to which there is a mismatch between the shape of the patient's face, and the seal-forming structure of the mass-manufactured patient interface, one or both must adapt in order for a seal to form.

One type of seal-forming structure extends around the periphery of the patient interface, and is intended to seal against the patient's face when force is applied to the patient interface with the seal-forming structure in confronting engagement with the patient's face. The seal-forming structure may include an air or fluid filled cushion, or a moulded or formed surface of a resilient seal element made of an elastomer such as a rubber. With this type of seal-forming structure, if the fit is not adequate, there will be gaps between the seal-forming structure and the face, and additional force will be required to force the patient interface against the face in order to achieve a seal.

Another type of seal-forming structure incorporates a flap seal of thin material positioned about the periphery of the mask so as to provide a self-sealing action against the face of the patient when positive pressure is applied within the mask. Like the previous style of seal forming portion, if the match between the face and the mask is not good, additional force may be required to achieve a seal, or the mask may leak. Furthermore, if the shape of the seal-forming structure does not match that of the patient, it may crease or buckle in use, giving rise to leaks.

Another type of seal-forming structure may comprise a friction-fit element, e.g. for insertion into a naris, however some patients find these uncomfortable.

Another form of seal-forming structure may use adhesive to achieve a seal. Some patients may find it inconvenient to constantly apply and remove an adhesive to their face.

A range of patient interface seal-forming structure technologies are disclosed in the following patent applications, assigned to ResMed Limited: WO 1998/004,310; WO 2006/074,513; WO 2010/135,785.

One form of nasal pillow is found in the Adam Circuit manufactured by Puritan Bennett. Another nasal pillow, or nasal puff is the subject of U.S. Pat. No. 4,782,832 (Trimble et al.), assigned to Puritan-Bennett Corporation.

ResMed Limited has manufactured the following products that incorporate nasal pillows: SWIFT™ nasal pillows mask, SWIFT™ II nasal pillows mask, SWIFT™ LT nasal pillows mask, SWIFT™ FX nasal pillows mask and MIRAGE LIBERTY™ full-face mask. The following patent applications, assigned to ResMed Limited, describe examples of nasal pillows masks: International Patent Application WO2004/073,778 (describing amongst other things aspects of the ResMed Limited SWIFT™ nasal pillows), US Patent Application 2009/0044808 (describing amongst other things aspects of the ResMed Limited SWIFT™ LT nasal pillows); International Patent Applications WO 2005/063,328 and WO 2006/130,903 (describing amongst other things aspects of the ResMed Limited MIRAGE LIBERTY™ full-face mask); International Patent Application WO 2009/052,560 (describing amongst other things aspects of the ResMed Limited SWIFT™ FX nasal pillows).

### 2.2.3.1.2 Positioning and Stabilising

A seal-forming structure of a patient interface used for positive air pressure therapy is subject to the corresponding force of the air pressure to disrupt a seal. Thus a variety of techniques have been used to position the seal-forming structure, and to maintain it in sealing relation with the appropriate portion of the face.

One technique is the use of adhesives. See for example US Patent Application Publication No. US 2010/0000534. However, the use of adhesives may be uncomfortable for some.

Another technique is the use of one or more straps and/or stabilising harnesses. Many such harnesses suffer from being one or more of ill-fitting, bulky, uncomfortable and awkward to use.

### 2.2.3.1.3 Pressurised Air Conduit

In one type of treatment system, a flow of pressurised air is provided to a patient interface through a conduit in an air circuit that fluidly connects to the patient interface so that, when the patient interface is positioned on the patient's face during use, the conduit extends out of the patient interface forwards away from the patient's face. This may sometimes be referred to as a “tube down” configuration.

Some patients find such interfaces to be unsightly or to create a feeling of claustrophobia and are consequently deterred from wearing them, reducing patient compliance. Additionally, conduits connecting to an interface at the front of a patient's face may sometimes be vulnerable to becoming tangled up in bed clothes.

### 2.2.3.1.4 Pressurised Air Conduit Used for Positioning/Stabilising the Seal-Forming Structure

An alternative type of treatment system which seeks to address these problems comprises a patient interface in which a tube that delivers pressurised air to the patient's airways also functions as part of the headgear to position and stabilise the seal-forming portion of the patient interface at the appropriate part of the patient's face. This type of patient interface may be referred to as having “conduit headgear” or “headgear tubing”. Such patient interfaces allow the conduit in the air circuit providing the flow of pressurised air from a respiratory pressure therapy device to connect to the patient interface in a position other than in front of the patient's face. One example of such a treatment system is disclosed in US Patent Publication No. US 2007/0246043, the contents of which are incorporated herein by reference, in which the conduit connects to a tube in the patient interface through a port positioned in use on top of the patient's head.

Patient interfaces incorporating headgear tubing may provide some advantages, for example avoiding a conduit connecting to the patient interface at the front of a patient's face, which may be unsightly and obtrusive. However, it is desirable for patient interfaces incorporating headgear tubing to be comfortable for a patient to wear over a prolonged duration when the patient is asleep, form an air-tight and stable seal with the patient's face, while also able to fit a range of patient head shapes and sizes.

2.2.3.2 Respiratory Pressure Therapy (rpt) Device

A respiratory pressure therapy (RPT) device may be used individually or as part of a system to deliver one or more of a number of therapies described above, such as by operating the device to generate a flow of air for delivery to an interface to the airways. The flow of air may be pressure-controlled (for respiratory pressure therapies) or flow-controlled (for flow therapies such as HFT). Thus RPT devices may also act as flow therapy devices. Examples of RPT devices include a CPAP device and a ventilator.

The designer of a device may be presented with an infinite number of choices to make. Design criteria often conflict, meaning that certain design choices are far from routine or inevitable. Furthermore, the comfort and efficacy of certain aspects may be highly sensitive to small, subtle changes in one or more parameters.

### 2.2.3.3 Air Circuit

An air circuit is a conduit or a tube constructed and arranged to allow, in use, a flow of air to travel between two components of a respiratory therapy system such as the RPT device and the patient interface. In some cases, there may be separate limbs of the air circuit for inhalation and exhalation. In other cases, a single limb air circuit is used for both inhalation and exhalation.

### 2.2.3.4 Humidifier

Delivery of a flow of air without humidification may cause drying of airways. The use of a humidifier with an RPT device and the patient interface produces humidified gas that minimizes drying of the nasal mucosa and increases patient airway comfort. In addition, in cooler climates, warm air applied generally to the face area in and about the patient interface is more comfortable than cold air.

### 2.2.3.5 Data Management

There may be clinical reasons to obtain data to determine whether the patient prescribed with respiratory therapy has been “compliant”, e.g. that the patient has used their RPT device according to one or more “compliance rules”. One example of a compliance rule for CPAP therapy is that a patient, in order to be deemed compliant, is required to use the RPT device for at least four hours a night for at least 21 of 30 consecutive days. In order to determine a patient's compliance, a provider of the RPT device, such as a health care provider, may manually obtain data describing the patient's therapy using the RPT device, calculate the usage over a predetermined time period, and compare with the compliance rule. Once the health care provider has determined that the patient has used their RPT device according to the compliance rule, the health care provider may notify a third party that the patient is compliant.

There may be other aspects of a patient's therapy that would benefit from communication of therapy data to a third party or external system.

Existing processes to communicate and manage such data can be one or more of costly, time-consuming, and error-prone.

### 2.2.3.6 Vent Technologies

Some forms of treatment systems may include a vent to allow the washout of exhaled carbon dioxide. The vent may allow a flow of gas from an interior space of a patient interface, e.g., the plenum chamber, to an exterior of the patient interface, e.g., to ambient.

### 2.2.4 Screening, Diagnosis, and Monitoring Systems

Polysomnography (PSG) is a conventional system for diagnosis and monitoring of cardio-pulmonary disorders, and typically involves expert clinical staff to apply the system. PSG typically involves the placement of 15 to 20 contact sensors on a patient in order to record various bodily signals such as electroencephalography (EEG), electrocardiogramalectromyography (EMG), etc. PSG for sleep disordered breathing has involved two nights of observation of a patient in a clinic, one night of pure diagnosis and a second night of titration of treatment parameters by a clinician. PSG is therefore expensive and inconvenient. In particular, it is unsuitable for home screening/diagnosis/monitoring of sleep disordered breathing.

Screening and diagnosis generally describe the identification of a condition from its signs and symptoms. Screening typically gives a true/false result indicating whether or not a patient's SDB is severe enough to warrant further investigation, while diagnosis may result in clinically actionable information.

Screening and diagnosis tend to be one-off processes, whereas monitoring the progress of a condition can continue indefinitely. Some screening/diagnosis systems are suitable only for screening/diagnosis, whereas some may also be used for monitoring.

Clinical experts may be able to screen, diagnose, or monitor patients adequately based on visual observation of PSG signals. However, there are circumstances where a clinical expert may not be available, or a clinical expert may not be affordable. Different clinical experts may disagree on a patient's condition. In addition, a given clinical expert may apply a different standard at different times.

### 3 BRIEF SUMMARY OF THE TECHNOLOGY

The present technology is directed towards providing medical devices used in the screening, diagnosis, monitoring, amelioration, treatment, or prevention of respiratory disorders having one or more of improved comfort, cost, efficacy, ease of use and manufacturability.

A first aspect of the present technology relates to apparatus used in the screening, diagnosis, monitoring, amelioration, treatment or prevention of a respiratory disorder.

Another aspect of the present technology relates to methods used in the screening, diagnosis, monitoring, amelioration, treatment or prevention of a respiratory disorder.

An aspect of certain forms of the present technology is to provide methods and/or apparatus that improve the compliance of patients with respiratory therapy.

Another aspect of one form of the present technology is a patient interface that is moulded or otherwise constructed with a perimeter shape which is complementary to that of an intended wearer.

3.1 Cushion having a Lattice Structure

Another aspect of the present technology comprises a patient interface comprising:
a plenum chamber pressurisable to a therapeutic pressure of at least 4 cmH₂O above ambient air pressure, said plenum chamber including a plenum chamber inlet port sized and structured to receive a flow of air at the therapeutic pressure for breathing by a patient;
a seal-forming structure constructed and arranged to form a seal with a region of the patient's face surrounding an entrance to the patient's airways, said seal-forming structure having a hole therein such that the flow of air at said therapeutic pressure is delivered to at least an entrance to the patient's nares, the seal-forming structure constructed and arranged to maintain said therapeutic pressure in the plenum chamber throughout the patient's respiratory cycle in use;
a vent to allow a continuous flow of gases exhaled by the patient from an interior of the plenum chamber to ambient, said vent being sized and shaped to maintain the therapeutic pressure in the plenum chamber in use; and
wherein the seal-forming structure comprises a cushion, the cushion being deformable and resilient and at least partially formed by a lattice structure;
wherein the patient interface is configured to allow the patient to breathe from ambient through their mouth in the absence of a flow of pressurised air through the plenum chamber inlet port, or the patient interface is configured to leave the patient's mouth uncovered in use.

In examples:
the seal-forming structure comprises a face engaging membrane configured to contact the patient's face, the face engaging membrane being flexible and resilient and at least partially covering the cushion in use;
the patient interface comprises a chassis portion at least partially forming the plenum chamber, the seal-forming structure being attached to and supported by the chassis portion, the chassis portion being stiffer than the seal-forming structure;
the face engaging membrane extends from the chassis portion;
the face engaging membrane is formed from an elastomeric material;
the cushion is positioned exterior to the plenum chamber;
the patient interface comprises a positioning and stabilising structure to provide a force to hold the seal-forming structure in a therapeutically effective position on the patient's head;
the chassis portion and seal-forming structure together form a cushion module removably attached to the positioning and stabilising structure;
the patient interface comprises a frame, the frame being configured to connect the positioning and stabilising structure to the cushion module;
the cushion module is removably attached to the frame;
the positioning and stabilising structure comprises a pair of gas delivery tubes configured to provide the flow of air at therapeutic pressure to the plenum chamber and configured to provide a force to hold the seal-forming structure in sealing position;
the cushion is formed flat and bent into a three-dimensional shape during assembly with the face engaging membrane; and/or
the cushion is formed in a three-dimensional shape.

In further examples:
the lattice structure is 3D printed;
the lattice structure is 3D printed in a shape corresponding to a unique patient's face;
the lattice structure is injection moulded;
the lattice structure is formed from TPU;
the lattice structure is formed from silicone;
the lattice structure is formed from a material having a Durometer hardness within the range of 20 Shore A to 80 Shore A;
the lattice structure comprises a two-dimensional structure;
the lattice structure comprises a three-dimensional structure;
the lattice structure comprises one of a fluorite structure, truncated cube structure, IsoTruss structure, hexagonal honeycomb structure, gyroid structure, and Schwarz structure;
the cushion is formed from foam having holes therein forming the lattice structure; and/or
the size, shape and/or spacing of the holes varies along a length of the cushion and/or between a first side of the cushion and a second side of the cushion.

In further examples:
the cushion comprises one or more characteristics that vary between different locations at which the seal-forming structure engages the patient's face;
the one or more characteristics of the cushion include stiffness of the cushion;
the one or more characteristics of the cushion include one or more characteristics of the lattice structure;
the one or more characteristics of the lattice structure include shape, thickness, density, spacing, relative orientation and/or material of unit cells forming the lattice structure;
the seal-forming structure is configured to seal to the patient's face at the patient's lip superior, on the lateral sides of the patient's nose and at the patient's nasal ridge;
the cushion comprises a lip superior portion provided within a portion of the seal-forming structure configured to seal to the patient's face at the patient's lip superior, the cushion comprises side of nose portions provided within respective portions of the seal-forming structure configured to seal to the patient's face at the sides of the patient's nose, and the cushion is stiffer at the side of nose portions than at the lip superior portion;
the cushion comprises a nasal ridge portion provided within a portion of the seal-forming structure configured to seal to the patient's face at the patient's nasal ridge and the cushion is stiffer at the side of nose portions than at the nasal ridge portion;
the seal-forming structure is configured to seal to the patient's face at the patient's lip inferior, at the patient's cheeks, on the lateral sides of the patient's nose and at the patient's nasal ridge;
the cushion comprises a lip inferior portion provided within a portion of the seal-forming structure configured to seal to the patient's face at the patient's lip inferior, the cushion comprises a pair of cheek portions provided within respective portions of the seal-forming structure configured to seal to the patient's face at the patient's cheeks, and the cushion is stiffer in the cheek portions than in the lip inferior portion;
the cushion comprises side of nose portions provided within respective portions of the seal-forming structure configured to seal to the patient's face on the lateral sides of the patient's nose, the cushion comprises a nasal ridge region provided within a portion of the seal-forming structure configured to seal to the patient's face at the patient's nasal ridge, and the cushion is stiffer in the side of nose portions than the nasal ridge portion;
the seal-forming structure is configured to seal to the patient's face at the patient's lip inferior, at the patient's cheeks, at the patient's lip superior, and at an inferior periphery of the patient's nose including the nasal alae and a pronasale region of the patient's nose;
the cushion comprises a lip inferior portion provided within a portion of the seal-forming structure configured to seal to the patient's face at the patient's lip inferior, the cushion comprises cheek portions provided within respective portions of the seal-forming structure configured to seal to the patient's face at the patient's cheeks, the cushion is stiffer in the cheek portions than in the lip inferior portion;
the cushion comprises a lip superior portion provided within a portion of the seal-forming structure configured to seal to the patient's face at the patient's lip superior, the cushion comprises an inferior nose periphery portion provided within a portion of the seal-forming structure configured to seal to the patient's face at the inferior periphery of the patient's nose, and the cushion is stiffer in the lip superior portion than in the inferior nose periphery portion;
the seal-forming structure is configured to seal to the patient's face at the patient's lip superior, between the nasal alae and the nasolabial sulci, and at an inferior periphery of the patient's nose including the nasal alae and a pronasale region of the patient's nose;
the cushion comprises a lip superior portion provided within a portion of the seal-forming structure configured to seal to the patient's face at the patient's lip superior, the cushion comprises a pair of posterior corner portions provided within a portion of the seal-forming structure configured to seal to the patient's face between the nasal alae and the nasolabial sulci, and the cushion is stiffer in the posterior corner portions than in the lip superior portion; and/or
the cushion comprises an inferior nose periphery portion provided within a portion of the seal-forming structure configured to seal to the patient's face at the inferior periphery of the patient's nose, and the cushion is stiffer in the posterior corner portions than in the inferior nose periphery portion.

In further examples:
the lattice structure comprises one or more characteristics that vary between a patient-facing side of the cushion corresponding to a side of the seal-forming structure configured to contact the patient's face in use and a non-patient facing side of the cushion corresponding to a side of the seal-forming structure configured to face away from the patient's face in use;
the lattice structure on the patient-facing side of the cushion is configured to avoid leaving red marks on the patient's face;
the lattice structure on the non-patient facing side of the cushion is configured to adapt readily to the shape of the patient's face;
the lattice structure comprises smaller unit cells on the patient-facing side than on the non-patient facing side;
the variation in the one or more characteristics of the lattice structure causes the cushion to be less stiff on the patient-facing side of the cushion than on the non-patient facing side of the cushion;
the material forming the unit cells of the lattice structure is thinner on the patient-facing side of the cushion than on the non-patient facing side of the cushion;
the material forming the unit cells of the lattice structure has a thickness within the range of 0.3-0.5 mm on the patient-facing side of the cushion;
the material forming the unit cells of the lattice structure has a thickness of within a range of 0.8-1.2 mm on the non-patient facing side of the cushion, such as 1 mm;
the lattice structure comprises one or more characteristics that vary along a length of the cushion, wherein in use the cushion receives a distributed load along said length of the cushion applied to a non-patient facing side of the cushion, and wherein due to the variation in the one or more characteristics the cushion applies a different distributed load to the patient's face along said length of the cushion;
the lattice structure comprises one or more characteristics that vary at and/or proximate a location corresponding to a sensitive facial feature on the patient's face;
the variation of the one or more characteristics causes the cushion to apply less pressure on the sensitive facial feature in use than would be applied without the variation of the one or more characteristics;
the variation of the one or more characteristics causes the cushion to apply less pressure on the sensitive facial feature in use than the cushion applies to the patient's face around the sensitive facial feature;
the variation of the one or more characteristics of the lattice structure results in lesser stiffness in the cushion at and/or proximate the location corresponding to the sensitive facial feature;
the cushion comprises a recess configured to be aligned in use with a sensitive facial feature on the patient's face, the recess shaped to receive the sensitive facial feature;
the recess is shaped to provide clearance between the cushion and the sensitive facial feature in an undeformed state;
the cushion comprises one or more force redistribution features configured to in use at least partially redirect forces received on a non-patient facing side of the cushion in a region of the cushion aligned with a sensitive facial feature into one or more regions of cushion alongside or spaced from the sensitive facial feature;
the one or more force redistribution features comprises a beam structure within the cushion positioned to in use span from a first region of the cushion located on a first side of the sensitive facial feature through a second region of the cushion overlying the sensitive facial feature and into a third region of the cushion on a second side of the sensitive facial feature;
at least one of the one or more force redistribution features comprises a stiffened region within the cushion being stiffer than one or more adjacent regions within the cushion, the stiffened region being positioned to in use span from a first region of the cushion located on a first side of the sensitive facial feature through a second region of the cushion overlying the sensitive facial feature and into a third region of the cushion on a second side of the sensitive facial feature, the stiffened region being stiffened by a variation in one or more characteristics of the lattice structure at the stiffened region;
the variation in one or more characteristics of the lattice structure includes variation in shape, thickness, density, spacing, relative orientation and/or material of unit cells forming the lattice structure;
the cushion is stiffer proximate the patient's face in the first region and in the third region than in the second region;
the sensitive facial feature is the patient's nose ridge;
a patient-facing side of the cushion is defined by unit cells of the lattice structure exposed to contact the face engaging membrane;
the cushion comprises a uniform surface on a patient-facing side of the cushion covering unit cells of the lattice structure; and/or
the uniform surface is integrally formed with unit cells of the lattice structure.

In further examples:
the cushion is removable from the patient interface;
at least some of the flow of air passing through the plenum chamber passes through the lattice structure forming the cushion;
the cushion forms a heat and moisture exchanger (HMX);
the cushion covers the plenum chamber inlet port; and/or
the cushion fills a majority of the plenum chamber.

In another aspect of the disclosed technology, a patient interface for delivering a flow of air to a patient for treatment of sleep disordered breathing comprises a plenum chamber and a seal-forming structure. The plenum chamber is pressurisable to a therapeutic pressure of at least 4 cmH₂O above ambient air pressure and includes a plenum chamber inlet port sized and structured to receive a flow of air at the therapeutic pressure for breathing by a patient. The seal-forming structure is constructed and arranged to form a seal with a region of the patient's face surrounding an entrance to the patient's airways, the seal-forming structure having a hole therein such that the flow of air at the therapeutic pressure is delivered to at least an entrance to the patient's nares, the seal-forming structure constructed and arranged to maintain the therapeutic pressure in the plenum chamber throughout the patient's respiratory cycle in use.

In an example, the seal-forming structure includes a cushion configured to be disposed between the plenum chamber and the patient's face in use, the cushion including a plurality of interconnected struts forming a plurality of voids.

In a further example, in use, when the seal-forming structure is in engagement with the patient's face, the struts are configured to flex thereby altering the size, shape and/or orientation of the voids to allow the cushion to conform to the patient's face.

In further examples: (a) the struts are resilient; (b) a characteristic of the cushion varies across the cushion such that in a first portion of the cushion the characteristic is different than in a second portion of the cushion, the first portion of the cushion having a level of flexibility that is different than the second portion of the cushion; (c) the characteristic of the cushion is 1) a thickness of the struts, 2) a density of the struts, 3) an orientation of the struts, 4) a spacing of the struts, 5) a size of the voids, 6) an orientation of the voids, and/or 7) a density of the voids; (d) the thickness of the struts in a first portion of the cushion is different than the thickness of the struts in a second portion of the cushion; (e) the size of the voids in the first portion of the cushion is different than the size of the voids in the second portion of the cushion; (f) the first portion of the cushion corresponds to a sensitive facial feature of the patient, and the second portion of the cushion does not correspond to a sensitive facial feature; (g) the sensitive facial feature is the patient's nasal ridge; (h) the first portion of the cushion has greater flexibility as compared to the second portion of the cushion; and/or (i) the struts and voids form a lattice structure.

In further examples: (a) the cushion is not formed from a foam material; and (b) the cushion is constructed from a foam material and has a plurality of macroscopic holes formed therein to form the voids.

In another aspect of the disclosed technology, a patient interface includes a seal-forming structure having a cushion, the cushion resembling bubble wrap.

In another aspect of the disclosed technology, a patient interface includes a seal-forming structure having a cushion, the cushion including a plurality of bladders (e.g., air-filled bladders).

In a further example, a plurality of hinge portions is interspersed between the bladders such that each bladder is movable relative to an adjacent bladder via a hinge portion.

In a further example, the hinge portions are thinned regions (e.g., living hinges).

In a further example, a stiffness or flexibility of the plurality of bladders may vary from bladder to bladder. In a further example, the stiffness or flexibility may vary by adjusting the amount of fluid in each bladder.

In another aspect of the disclosed technology, a patient interface includes a seal-forming structure having a cushion, the cushion including a plurality of relatively flexible, relatively thin hinge portions interspersed between relatively stiff portions. In a further example, the hinge portions and the relatively stiff portions form a grid.

### 3.2 Automatic Sizing

One form of the present technology comprises automatic sizing of a component of a patient interface which interfaces with the patient's face (also referred to as “facial interface” hereinafter) without the assistance of a trained individual or others.

Another aspect of one form of the present technology is the automatic measurement of a subject's (e.g. a patient's/user's) facial features based on data collected from the user.

Another aspect of one form of the present technology is the automatic recommendation of a facial interface size based on a comparison between data collected from a user to a corresponding data record.

Another aspect of one form of the present technology is the automatic recommendation of a customized facial interface size based on a data collected from a user. The customized facial interface may be unique to a given user based on his/her facial geometry.

Another aspect of one form of the present technology is a mobile application that conveniently determines an appropriate facial interface size for a particular user based on a two-dimensional image.

Another aspect of one form of the present technology is a mobile application that conveniently determines an appropriate facial interface size for a particular user based on a three-dimensional image.

Some versions of the present technology include automated method(s) for selecting a facial interface according to facial interface size. The method(s) may operate in one or more processors. The method may include receiving image data captured by an image sensor. The captured image data may contain one or more facial features of an intended user of the facial interface in association with a predetermined reference feature having a known dimension. The method may include detecting one or more facial features of the user in the captured image data. The method may include detecting the predetermined reference feature in the captured image data. The method may include processing image pixel data of the image to measure an aspect of the one or more facial features detected in the image based on the predetermined reference feature. The method may include selecting a facial interface size from a group of standard facial interface sizes based on a comparison between the measured aspect of the one or more facial features and a data record relating sizing information of the group of standard facial interface sizes and the measured aspect of the one or more facial features.

In some versions, the aspect of the one or more facial features may include a distance between a sellion and supramenton of the user. The method may include calculating a value of the measured aspect based on a scaling factor derived from the reference feature. The method may include adjusting a value of the measured aspect with an anthropometric correction factor. The anthropometric correction factor may be calculated based on facial interface return data. The method may include calculating the scaling factor as a function of the known dimension of the predetermined reference feature and a detected pixel count for the detected reference feature. The predetermined reference feature may be a coin. The detecting the reference feature may include applying a cascade classifier to the captured image data. The method may include calculating a value of the measured aspect based on a scaling factor derived from the coin. The method may include calculating the scaling factor as a function of the known dimension of the coin in the captured image data and a detected pixel count for the coin that is detected. The detected pixel count for the coin that is detected may be a width of an ellipse fitted to the coin. The predetermined reference feature may be a cornea or iris of the user.

In some versions, the method may include, for image capture, displaying the reference feature on a display interface of a display device coupled with the image sensor. The display interface may include a targeting guide and a live action preview of content detected by the image sensor. The content may include the reference feature as displayed on the display interface. The method may include controlling capturing of the image data to satisfy at least one alignment condition. The at least one alignment condition may include detection of positioning of the reference feature of the live action preview within a box of the targeting guide. The at least one alignment condition may include detection of a tilt condition being within about +/−10 degrees of a superior-inferior extending axis. The at least one alignment condition may include detection of a tilt condition being within about +/−5 degrees of a superior-inferior extending axis. Detection of a tilt condition may be performed by reading an inertial measurement unit (IMU).

In some versions, the predetermined reference feature may be a QR code. Optionally, the processing image pixel data may include counting pixels. The method may include generating an automated electronic offer for purchase and/or automated shipment instructions for a facial interface based on the selected facial interface size. The method may include calculating an average of the measured aspect of the facial feature from a plurality of captured images of the one or more facial features. Optionally, the method may include automatic recommendation of a customized facial interface size based on a data collected from a user and the customized facial interface may be unique to a given user based on his/her facial geometry.

Some versions of the present technology include a system(s) for automatically recommending a facial interface size complementary to a particular user's facial features. The system(s) may include one or more servers. The one or more servers may be configured to communicate with a computing device over a network. The one or more servers may be configured to receive image data captured by an image sensor, where the captured image data may contain one or more facial features of an intended user of the facial interface in association with a predetermined reference feature having a known dimension. The one or more servers may be configured to detect one or more facial features of the user in the captured image data. The one or more servers may be configured to detect the predetermined reference feature in the captured image data. The one or more servers may be configured to process image pixel data of the image to measure an aspect of the one or more facial features detected in the image based on the predetermined reference feature. The one or more servers may be configured to select a facial interface size from a group of standard facial interface sizes based on a comparison between the measured aspect of the one or more facial features and a data record relating sizing information of the group of standard facial interface sizes and the measured aspect of the one or more facial features.

In some versions, the aspect of the one or more facial features may include a distance between a sellion and supramenton of the user. The one or more servers may be configured to calculate a value of the measured aspect based on a scaling factor derived from the reference feature. The one or more servers may be configured to adjust a value of the measured aspect with an anthropometric correction factor. The anthropometric correction factor may be calculated based on facial interface return data. The one or more servers may be configured to calculate the scaling factor as a function of the known dimension of the predetermined reference feature and a detected pixel count for the detected reference feature. The predetermined reference feature may include a coin. The one or more servers may be configured to detect the reference feature by applying a cascade classifier to the captured image data. The one or more servers may be further configured to calculate a value of the measured aspect based on a scaling factor derived from the coin. The one or more servers may be configured to calculate the scaling factor as a function of the known dimension of the coin in the captured image data and a detected pixel count for the coin that is detected. The detected pixel count for the coin that is detected may be a width of an ellipse fitted to the coin. The predetermined reference feature may be a cornea of the user.

In some versions, the system may include the computing device. The computing devices may be configured to, for image capture, generate a display of the reference feature on a display interface of a display device that may be coupled with the image sensor. The display interface may include a targeting guide and a live action preview of content detected by the image sensor. The content may include the reference feature as displayed on the display interface. The computing device may be further configured to control capturing of the image data to satisfy at least one alignment condition. The at least one alignment condition may include detection of positioning of the reference feature of the live action preview within a box of the targeting guide. The at least one alignment condition may include detection of a tilt condition being within about +/−10 degrees of a superior-inferior extending axis. The at least one alignment condition may include detection of a tilt condition being within about +/−5 degrees of a superior-inferior extending axis. The detection of a tilt condition may be performed by reading an inertial measurement unit (IMU).

In some versions, the predetermined reference feature may include a QR code. In some cases, to process image pixel data, the one or more servers may be configured to count pixels. The one or more servers may be configured to generate an automated electronic offer for purchase and/or automated shipment instructions for a facial interface based on the selected facial interface size. The one or more servers may be configured to calculate an average of the measured aspect of the facial feature from a plurality of captured images of the facial features. The one or more servers may be configured to communicate the selected facial interface size to the computing device over the network. Optionally, the server may be configured to automatically recommend a customized facial interface size based on a data collected from a user and the customized facial interface may be unique to a given user based on his/her facial geometry.

Some versions of the present technology include a system(s) for automatically recommending a facial interface size complementary to a particular user's facial features. The system(s) may include a mobile computing device. The mobile computing device may be configured to communicate with one or more servers over a network. The mobile computing device may be configured to receive captured image data of an image. The captured image data may contain one or more facial features of a user in association with a predetermined reference feature having a known dimension. The image data may be captured with an image sensor. The mobile computing device may be configured to detect one or more facial features of the user in the captured image data. The mobile computing device may be configured to detect the predetermined reference feature in the captured image data. The mobile computing device may be configured to process image pixel data of the image to measure an aspect of the one or more facial features detected in the image based on the predetermined reference feature. The mobile computing device may be configured to select a facial interface size from a group of standard facial interface sizes based on a comparison between the measured aspect of the one or more facial features and a data record relating sizing information of the group of standard facial interface sizes and the measured aspect of the one or more facial features.

In some versions, the aspect of the one or more facial features may include a distance between a sellion and supramenton of the user. The mobile computing device may be configured to calculate a value of the measured aspect based on a scaling factor derived from the reference feature. The mobile computing device may be further configured to adjust a value of the measured aspect with an anthropometric correction factor. The anthropometric correction factor may be calculated based on facial interface return data. The mobile computing device may be configured to calculate the scaling factor as a function of the known dimension of the predetermined reference feature and a detected pixel count for the detected reference feature. The predetermined reference feature may be a coin. The mobile computing device may be configured to detect the reference feature by applying a cascade classifier to the captured image data. The mobile computing device may be configured to calculate a value of the measured aspect based on a scaling factor derived from the coin. The mobile computing device may be configured to calculate the scaling factor as a function of the known dimension of the coin in the captured image data and a detected pixel count for the coin that is detected. The detected pixel count for the coin that is detected may be a width of an ellipse fitted to the coin. In some versions, the predetermined reference feature may be a cornea or iris of the user.

The mobile computing device may be configured to, for the image capture, generate a display of the reference feature on a display interface of a display device that may be coupled with the image sensor. The display interface may include a targeting guide and a live action preview of content detected by the image sensor. The content may include the reference feature as displayed on the display interface. The mobile computing device may be configured to control capturing of the image data to satisfy at least one alignment condition. The at least one alignment condition may include detection of positioning of the reference feature of the live action preview within a box of the targeting guide. The at least one alignment condition may include detection of a tilt condition being within about +/−10 degrees of a superior-inferior extending axis. The at least one alignment condition may include detection of a tilt condition being within about +/−5 degrees of a superior-inferior extending axis. Detection of a tilt condition may be performed by reading an inertial measurement unit (IMU).

In some versions, the predetermined reference feature may be a QR code. In some cases, to process image pixel data, the mobile computing device may be configured to count pixels. The mobile computing device may be configured to request an automated electronic offer for purchase and/or automated shipment instructions for an interface based on the selected facial interface size. The mobile computing device may be configured to calculate an average of the measured aspect of the facial feature from a plurality of captured images of the facial features. The mobile computing device may be configured to communicate the selected facial interface size to a server over the network. Optionally, the mobile phone may be configured to automatic recommend a customized facial interface size based on a data collected from a user, where the customized facial interface may be unique to a given user based on his/her facial geometry.

Some versions of the present technology include apparatus for automatically recommending a facial interface size complementary to a particular user's facial features. The apparatus may include means for receiving image data captured by an image sensor. The captured image data may contain one or more facial features of an intended user of the facial interface in association with a predetermined reference feature having a known dimension. The apparatus may include means for detecting one or more facial features of the user in the captured image data. The apparatus may include means for detecting the predetermined reference feature in the captured image data. The apparatus may include means for processing image pixel data of the image to measure an aspect of the one or more facial features detected in the image based on the predetermined reference feature. The apparatus may include means for selecting a facial interface size from a group of standard facial interface sizes based on a comparison between the measured aspect of the one or more facial features and a data record relating sizing information of the group of standard facial interface sizes and the measured aspect of the one or more facial features.

### 3.3 Personalisation

An aspect of one form of the present technology is a processor-implemented method for producing a lattice structure of a customised patient interface component, the method comprising:
receiving, using communication circuitry, data representative of one or more landmark features of a head of a human;
identifying, using at least one processor, one or more landmark feature locations of the landmark features based on the data;
determining, using the at least one processor, a set of manufacturing specifications for production of the lattice structure of the patient interface component based on the one or more landmark feature locations; and
controlling one or more manufacturing machines to produce the lattice structure of the patient interface component based on the set of manufacturing specifications.

An aspect of one form of the present technology is a processor-implemented method for producing a lattice structure of a customised patient interface component, the method comprising:
receiving, using communication circuitry, data representative of one or more landmark features of a head of a human;
identifying, using at least one processor, one or more landmark feature locations of the landmark features based on the data;
determining, using the at least one processor, a set of manufacturing specifications for production of the lattice structure of the patient interface component based on the one or more landmark feature locations; and
causing one or more manufacturing machines to produce the lattice structure of the patient interface component based on the set of manufacturing specifications.

In examples: (a) the data is representative of one or more landmark features of a head of an intended user of the patient interface; (b) the data comprises image data; (c) at least a portion of the image data is captured by an image sensor (d) the method comprises the step of capturing at least a portion of the image data with an image sensor; (e) the data comprises two-dimensional image data; and/or (f) the data comprises three-dimensional image data.

In an example, causing one or more manufacturing machines to produce the lattice structure of the patient interface component includes, controlling the one or more manufacturing machines to produce the lattice structure of the patient interface component based on the set of manufacturing specifications.

In an example, the method is performed by a manufacturing system including the at least one processor and the communication circuitry.

In examples the method comprises; (a) the step of capturing at least a portion of the data with an image sensor; and/or (b) the step of identifying at least one relationship between two or more of the landmark feature locations, wherein determining the set of manufacturing specifications is based at least in part on the at least one relationship between the two or more of the landmark feature locations.

In examples identifying the at least one relationship between the two or more of the landmark feature locations comprises determining distance between two or more of: a subnasale, a sellion, a tragion, a posterior-most point of the head, a superior-most point of the head, a lateral-most point of the right orbital margin, a lateral-most point of the left orbital margin, an inferior-most point of the orbital margin, the Frankfort horizontal plane, and a coronal plane aligned with the tragion.

In examples identifying the at least one relationship between the two or more of the landmark feature locations comprises: (a) determining a distance in the sagittal plane between the subnasale and the tragion; (b) determining a vertical distance in the sagittal plane between the subnasale and the sellion; (c) determining a distance between the subnasale and the coronal plane aligned with the tragion, the distance being normal to said coronal plane; (d) determining a distance between the lateral-most point of the left or right orbital margin and the coronal plane aligned with the tragion, the distance being normal to said coronal plane; (e) determining a vertical distance between the subnasale and the superior-most point of the head; (f) determining a vertical distance between the superior-most point of the head and the Frankfort horizontal plane; (g) determining a distance between the rearmost point of the head and a coronal plane aligned with the tragion, the distance being normal to said coronal plane; and/or (h) determining a distance between the lateral-most points of the left and right orbital margins.

In examples: (a) the method comprises the step of determining at least one performance requirement for the lattice structure of the patient interface component based on the one or more landmark feature locations; (b) the at least one performance requirement comprises one or more of: stiffness, contact pressure, compliance, a force to be applied by or to the component, elasticity, dimensions and positioning on the head; (c) the lattice structure of the patient interface component comprises a plurality of regions, and at least one performance requirement is determined for each region; (d) the at least one performance requirement is determined based at least in part on properties of another component of the patient interface intended for use with the customised patient interface component; and/or (e) determining the set of manufacturing specifications is based at least in part on the at least one performance requirement.

In some examples the step of determining the at least one performance requirement comprises receiving and analysing facial movement data representing changes in shape and/or size of the patient's face during facial movement.

In examples the set of manufacturing specifications comprises: (a) at least one material specification; (b) at least one construction technique specification; and/or (c) at least one dimension specification.

In examples, determining the set of manufacturing specifications comprises: (a) selecting the set of manufacturing specifications from a plurality of pre-existing sets of manufacturing specifications; (b) selecting the set of pre-existing manufacturing specifications is based on a comparison between the one or more landmark feature locations determined for the human, and one or more landmark feature locations associated with the set of pre-existing manufacturing specifications; and/or (c) selecting a plurality of manufacturing specifications to form the set of manufacturing specifications from a plurality of pre-existing manufacturing specifications.

In examples, the method comprises producing manufacturing machine programming instructions for production of the lattice structure of the patient interface component based on the set of manufacturing specifications. In examples, producing the lattice structure of the patient interface component comprises programming at least one manufacturing machine with the manufacturing machine programming instructions, and operating the at least one manufacturing machine according to the manufacturing machine programming instructions.

In examples: (a) producing the manufacturing machine programming instructions comprises generating a map representing the set of manufacturing specifications, and generating the manufacturing machine programming instructions based on the map; and/or (b) producing the manufacturing machine programming instructions comprises generating a model of the lattice structure of the patient interface component based on the set of manufacturing specifications, and generating the manufacturing machine programming instructions based on the model.

In examples, producing the lattice structure of the patient interface component comprises (a) additive manufacturing of the lattice structure; (b) 3D printing the lattice structure; (c) laser cutting the lattice structure; (d) knitting the lattice structure; (e) weaving the lattice structure; and/or (f) generating instructions for one or more manufacturing apparatuses configured to produce the lattice structure of controlling the one or more manufacturing apparatuses to produce the lattice structure based on the generated instructions.

In an example the patient interface component comprises a cushion of a seal-forming structure of the patient interface.

An aspect of one form of the present technology is a system for producing a lattice structure of a customised patient interface component, the system comprising:
one or more processors for receiving data representative of one or more landmark features of a human;
the one or more processors further configured to identify one or more landmark feature locations of the landmark features based on the data;
the one or more processors further configured to determine a set of manufacturing specifications for production of the lattice structure of the patient interface component based on the one or more landmark feature locations; and
at least one manufacturing machine configured to produce the lattice structure of the patient interface component based on the set of manufacturing specifications.

An aspect of one form of the present technology is a processor-implemented method performed by a processing system including at least one processor and communication circuitry for production of a lattice structure of a patient interface component, the method comprising:
receiving, using the communication circuitry, data representative of one or more landmark features of a head of a human;
identifying, using the processing system, one or more landmark feature locations of the landmark features based on the data;
determining, using the processing system, a set of manufacturing specifications for production of the lattice structure of the patient interface component based on the one or more landmark feature locations; and
communicating, using the communication circuitry, the set of manufacturing specifications to a manufacturing system comprising at least one manufacturing machine configured to produce the lattice structure of the patient interface component based on the set of manufacturing specifications.

An aspect of one form of the present technology is a system for producing a lattice structure of a customised patient interface component, the system comprising:
one or more processors for receiving data representative of one or more landmark features of a head of a human;
the one or more processors further configured to identify one or more landmark feature locations of the landmark features based on the data;
the one or more processors further configured to determine a set of manufacturing specifications for production of the lattice structure of the patient interface component based on the one or more landmark feature locations; and
the one or more processors further configured to communicate the set of manufacturing specifications to a manufacturing system comprising at least one manufacturing machine configured to produce the lattice structure of the patient interface component based on the set of manufacturing specifications.

An aspect of one form of the present technology is a processor-implemented method performed by a processing system including at least one processor and communication circuitry for production of a lattice structure of a patient interface component, the method comprising:
receiving, using the communication circuitry, data representative of one or more landmark feature locations of landmark features of a head of a human;
determining, using the processing system, a set of manufacturing specifications for production of the lattice structure of the patient interface component based on the one or more landmark feature locations; and
communicating, using the communication circuitry, the set of manufacturing specifications to a manufacturing system comprising at least one manufacturing machine configured to produce the lattice structure of the patient interface component based on the set of manufacturing specifications.

An aspect of one form of the present technology is a system for producing a lattice structure of a customised patient interface component, the system comprising:
one or more processors for receiving one or more landmark feature locations of landmark features of a head of a human, the one or more landmark feature locations identified from data representative of the one or more landmark features of the head;
the one or more processors further configured to determine a set of manufacturing specifications for production of the lattice structure of the patient interface component based on the one or more landmark feature locations; and
the one or more processors further configured to communicate the set of manufacturing specifications to a manufacturing system comprising at least one manufacturing machine configured to produce the lattice structure of the patient interface component based on the set of manufacturing specifications.

An aspect of one form of the present technology is a processor-implemented method for production of a lattice structure of a patient interface component, the method comprising:
receiving, using communication circuitry, a set of manufacturing specifications for production of the lattice structure of the patient interface component, wherein the set of manufacturing specifications are determined based on one or more landmark feature locations identified from data representative of one or more landmark features of a head of a human; and
controlling one or more manufacturing machines to produce the lattice structure of the patient interface component using based on the set of manufacturing specifications.

An aspect of one form of the present technology is a processor-implemented method for production of a lattice structure of a patient interface component, the method comprising:
receiving, using communication circuitry, a set of manufacturing specifications for production of the lattice structure of the patient interface component, wherein the set of manufacturing specifications are determined based on one or more landmark feature locations identified from data representative of one or more landmark features of a head of a human; and
causing one or more manufacturing machines to produce the lattice structure of the patient interface component using based on the set of manufacturing specifications.

In one example, causing one or more manufacturing machines to produce the lattice structure of the patient interface component includes controlling the one or more manufacturing machines to produce the lattice structure of the patient interface component.

An aspect of one form of the present technology is a system for producing a lattice structure of a customised patient interface component, the system comprising:
one or more processors for receiving a set of manufacturing specifications for production of the lattice structure of the patient interface component, wherein the set of manufacturing specifications are determined based on one or more landmark feature locations identified from data representative of one or more landmark features of a head of a human; and
at least one manufacturing machine configured to produce the lattice structure of the patient interface component based on the set of manufacturing specifications.

An aspect of one form of the present technology is a processor-implemented method for production of a lattice structure of a patient interface component, the method comprising:
obtaining, based on data received from a device using communication circuitry, information representative of one or more landmark feature locations for a human head;
determining, using at least one processor, a set of manufacturing specifications for production of the lattice structure of the patient interface component based on the one or more landmark feature locations; and
causing one or more manufacturing machines to produce the lattice structure of the patient interface component based on the set of manufacturing specifications.

An aspect of one form of the present technology is a system for producing a lattice structure of a patient interface component, the system comprising:
one or more processors for obtaining information representative of one or more landmark feature locations for a human head;
the one or more processors further configured to determine a set of manufacturing specifications for production of the lattice structure of a patient interface component based on the one or more landmark feature locations; and
the one or more processors further configured to produce the lattice structure of the patient interface component based on the set of manufacturing specifications.

An aspect of one form of the present technology is an apparatus for producing a lattice structure of a patient interface component, the apparatus comprising:
means for obtaining information representative of one or more landmark feature locations for a human's head;
means for determining a set of manufacturing specifications for production of the lattice structure of the patient interface component based on the one or more landmark feature locations; and
means for producing the lattice structure of the patient interface component based on the set of manufacturing specifications.

In examples, the patient interface component comprises: (a) a cushion for a seal-forming structure of the patient interface.

Another form of the present technology comprises a cushion for a patient interface produced by any one of the above methods and/or systems.

Another form of the present technology comprises a cushion for a patient interface, the cushion comprising a lattice structure being formed by 3D printing based on instructions generated based on identification of facial landmarks and/or distances between said landmarks.

The methods, systems, devices and apparatus described may be implemented so as to improved comfort, cost, efficacy, ease of use and manufacturability of customized patient interface and/or component thereof.

The methods, systems, devices and apparatus described may be implemented so as to improve the functionality of a processor, such as a processor of a specific purpose computer used to identify landmark features and/or their locations, identifying relationships between the landmark features, determining functional requirements (e.g., for a patient interface and/or one or more components thereof), determining manufacturing specifications, and/or producing or generating manufacturing machine programmable instructions. Moreover, the described methods, systems, devices and apparatus can provide improvements in the technological field of automated generation of machine programming instructions for producing a customized patient interface and/or component thereof. Moreover, the described methods systems, devices and apparatus provide increased flexibility in producing customized patient interface and/or component thereof that will properly fit a user and provide the most comfort, and/or faster production of the customized patient interface and/or component thereof. Examples of the present technology provide customized patient interface and/or component thereof faster than conventional methods (e.g., from the time they are requested) and/or with accuracy that cannot be provided by conventional methods, at least because a patient, clinician and/or manufacturer cannot accurately consider and implement all of the factors that go into providing a customized patient interface and/or component thereof with accuracy, improved comfort and/or without significant cost and/or time.

An aspect of certain forms of the present technology is a medical device that is easy to use, e.g. by a person who does not have medical training, by a person who has limited dexterity, vision or by a person with limited experience in using this type of medical device.

An aspect of one form of the present technology is a portable RPT device that may be carried by a person, e.g., around the home of the person.

An aspect of one form of the present technology is a patient interface that may be washed in a home of a patient, e.g., in soapy water, without requiring specialised cleaning equipment. An aspect of one form of the present technology is a humidifier tank that may be washed in a home of a patient, e.g., in soapy water, without requiring specialised cleaning equipment.

The methods, systems, devices and apparatus described may be implemented so as to improve the functionality of a processor, such as a processor of a specific purpose computer, respiratory monitor and/or a respiratory therapy apparatus. Moreover, the described methods, systems, devices and apparatus can provide improvements in the technological field of automated management, monitoring and/or treatment of respiratory conditions, including, for example, sleep disordered breathing.

Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 4 BRIEF DESCRIPTION OF THE DRAWINGS

The present technology is illustrated by way of example, and not by way of limitation, in the figures of the accompanying drawings, in which like reference numerals refer to similar elements including:

### 4.1 Respiratory Therapy Systems

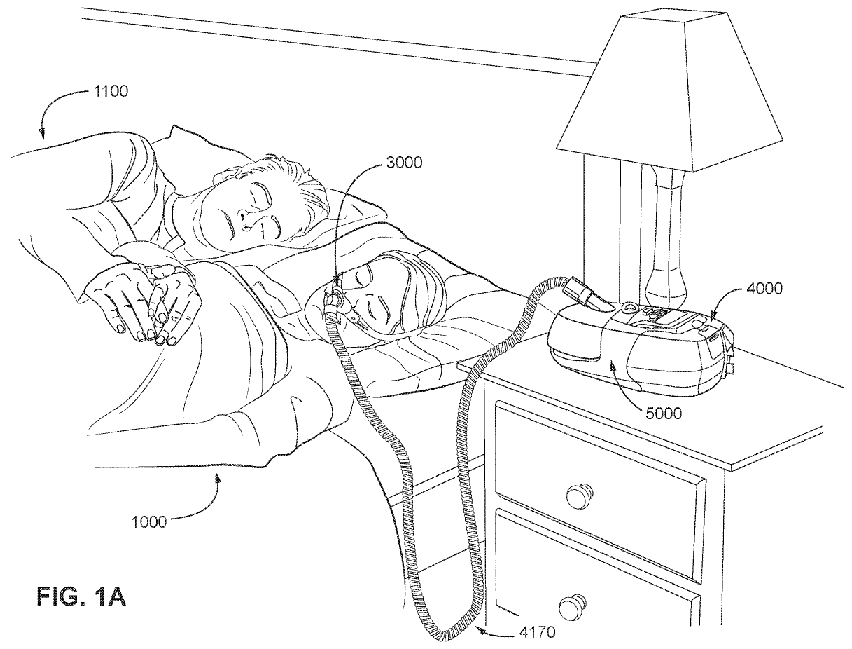

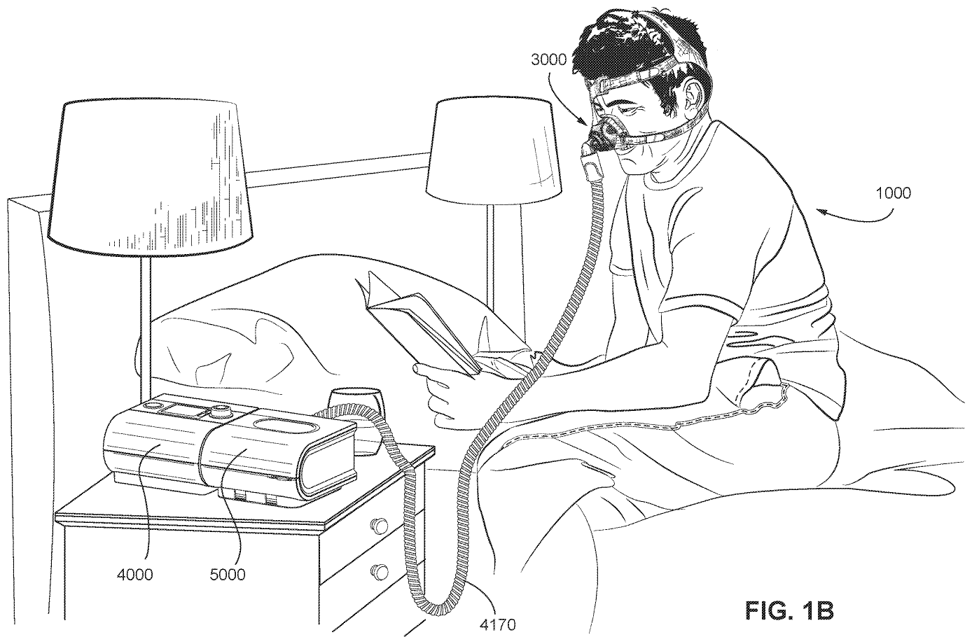

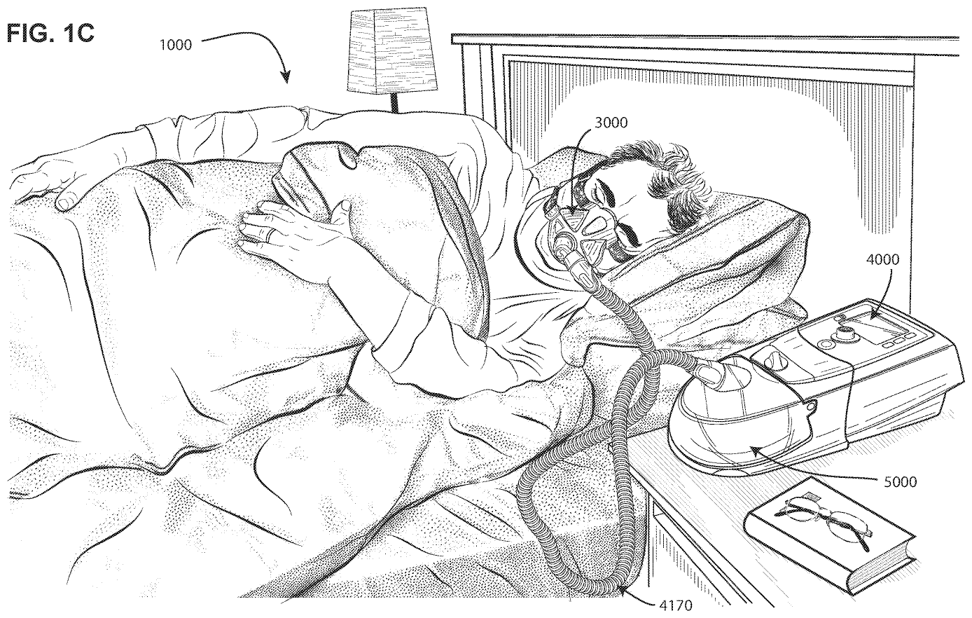

FIG. 1A shows a system including a patient 1000 wearing a patient interface 3000, in the form of nasal pillows, receiving a supply of air at positive pressure from an RPT device 4000. Air from the RPT device 4000 is humidified in a humidifier 5000, and passes along an air circuit 4170 to the patient 1000. A bed partner 1100 is also shown. The patient is sleeping in a supine sleeping position.

FIG. 1B shows a system including a patient 1000 wearing a patient interface 3000, in the form of a nasal mask, receiving a supply of air at positive pressure from an RPT device 4000. Air from the RPT device is humidified in a humidifier 5000, and passes along an air circuit 4170 to the patient 1000.

FIG. 1C shows a system including a patient 1000 wearing a patient interface 3000, in the form of a full-face mask, receiving a supply of air at positive pressure from an RPT device 4000. Air from the RPT device is humidified in a humidifier 5000, and passes along an air circuit 4170 to the patient 1000. The patient is sleeping in a side sleeping position.

### 4.2 Respiratory System and Facial Anatomy

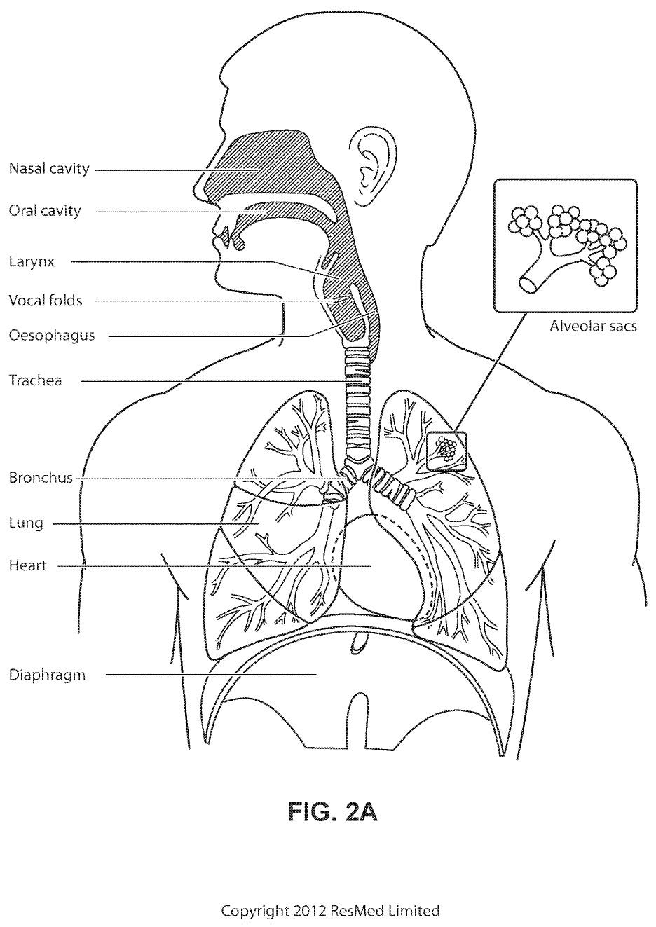

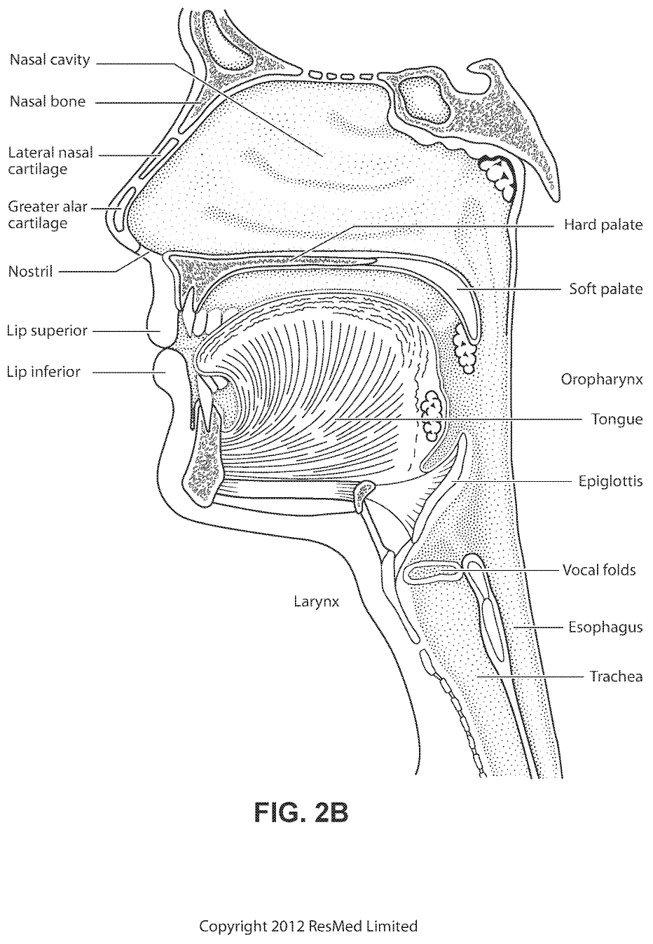

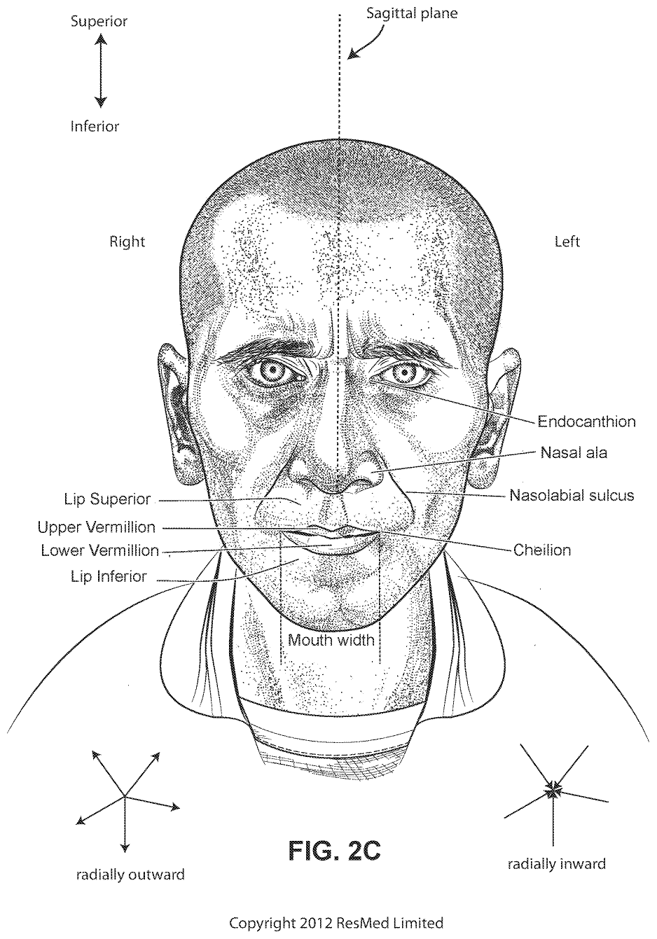

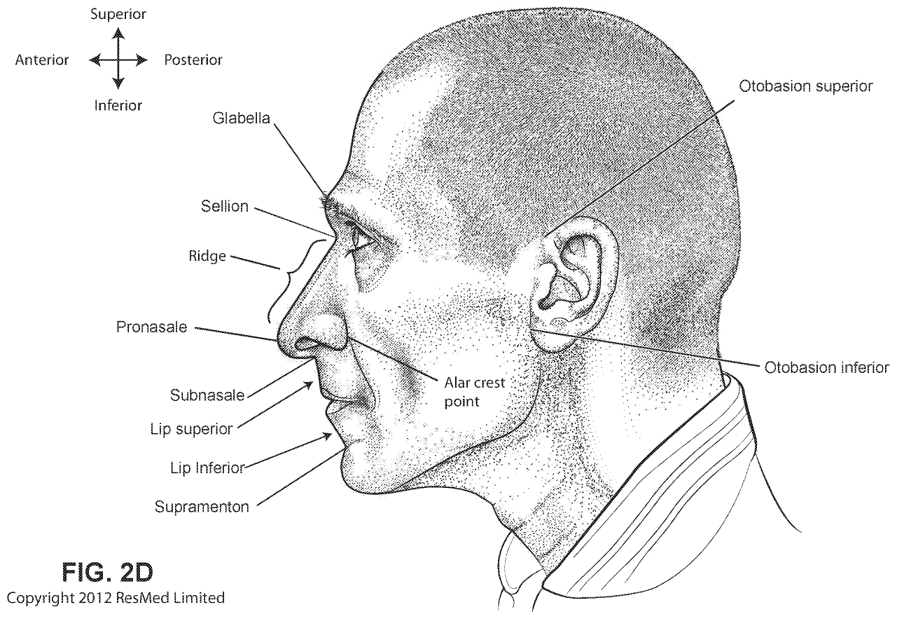

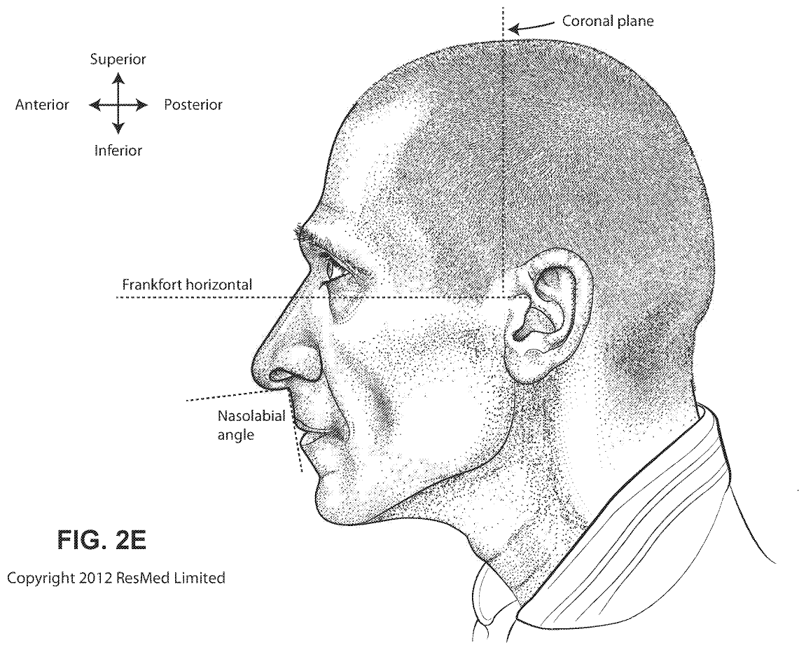

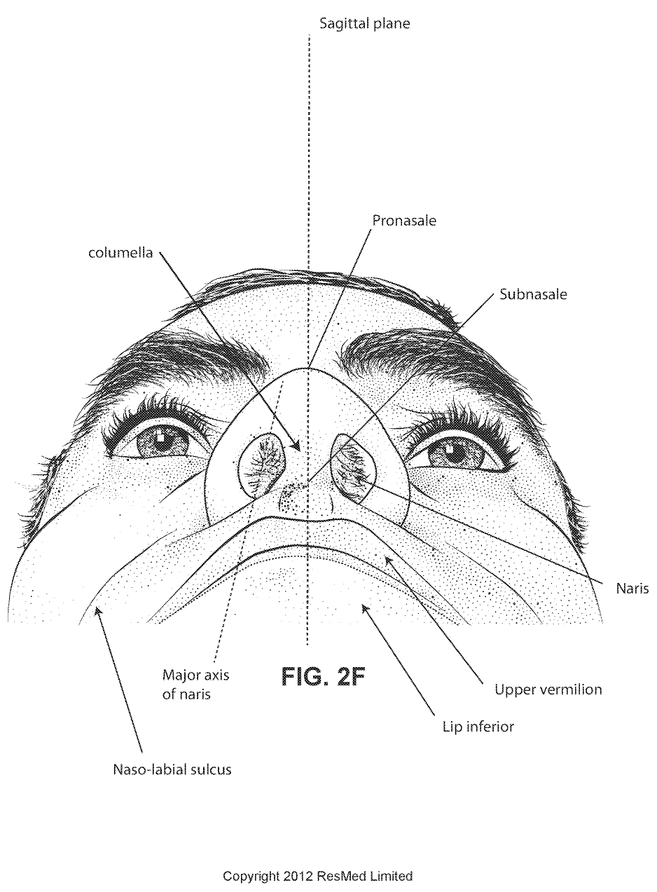

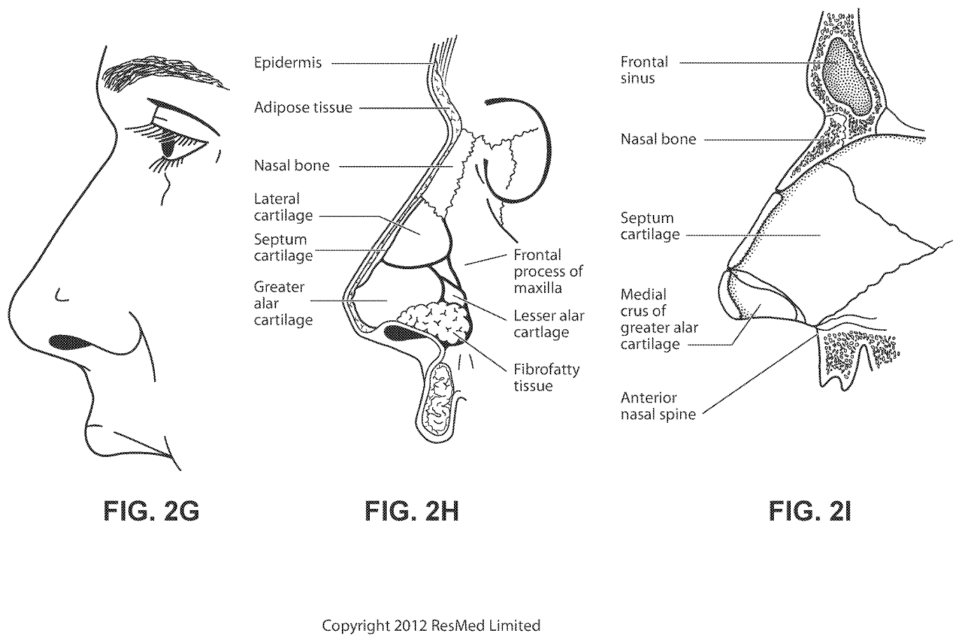

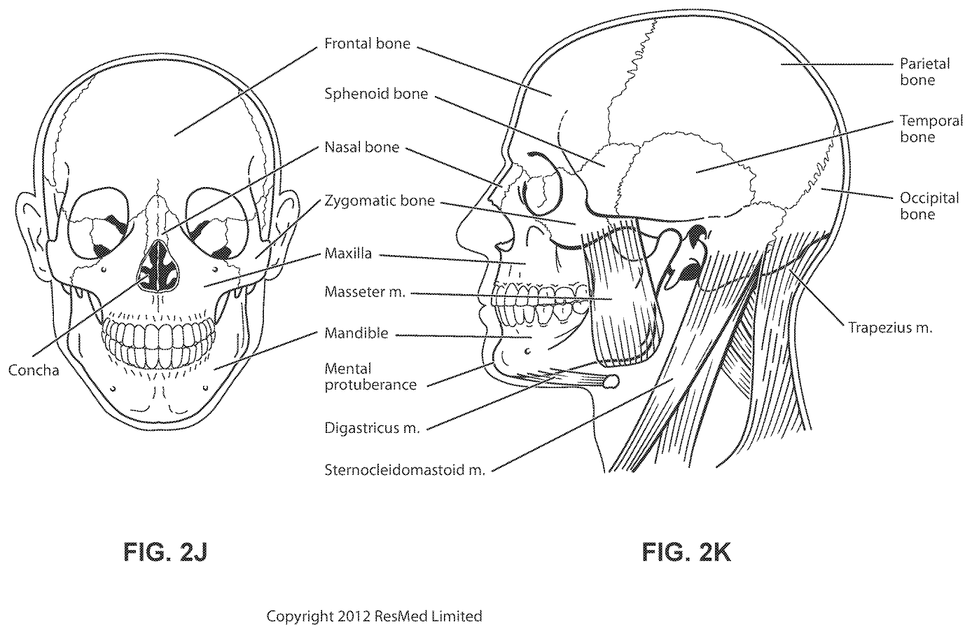

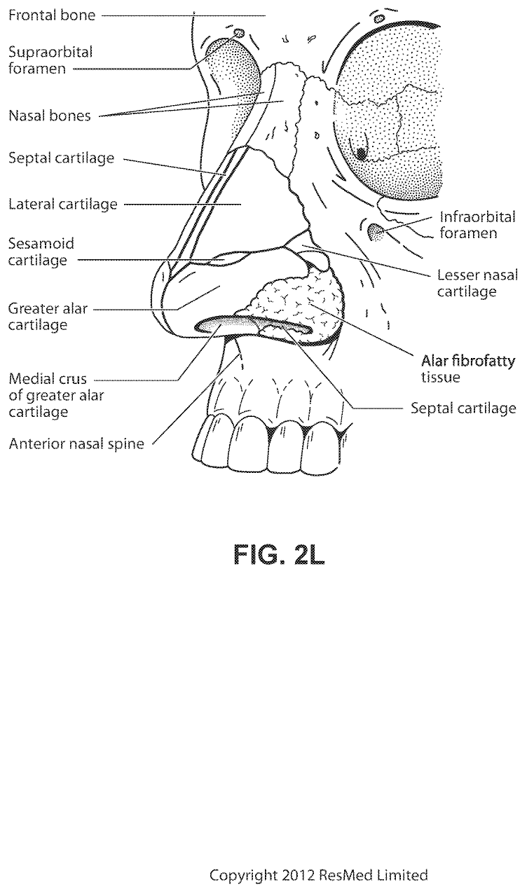

FIG. 2A shows an overview of a human respiratory system including the nasal and oral cavities, the larynx, vocal folds, oesophagus, trachea, bronchus, lung, alveolar sacs, heart and diaphragm.

FIG. 2B shows a view of a human upper airway including the nasal cavity, nasal bone, lateral nasal cartilage, greater alar cartilage, nostril, lip superior, lip inferior, larynx, hard palate, soft palate, oropharynx, tongue, epiglottis, vocal folds, oesophagus and trachea.

FIG. 2C is a front view of a face with several features of surface anatomy identified including the lip superior, upper vermilion, lower vermilion, lip inferior, mouth width, endocanthion, a nasal ala, nasolabial sulcus and cheilion. Also indicated are the directions superior, inferior, radially inward and radially outward.

FIG. 2D is a side view of a head with several features of surface anatomy identified including glabella, sellion, pronasale, subnasale, lip superior, lip inferior, supramenton, nasal ridge, alar crest point, otobasion superior and otobasion inferior. Also indicated are the directions superior & inferior, and anterior & posterior.

FIG. 2E is a further side view of a head. The approximate locations of the Frankfort horizontal and nasolabial angle are indicated. The coronal plane is also indicated.

FIG. 2F shows a base view of a nose with several features identified including naso-labial sulcus, lip inferior, upper Vermilion, naris, subnasale, columella, pronasale, the major axis of a naris and the midsagittal plane.

FIG. 2G shows a side view of the superficial features of a nose.

FIG. 2H shows subcutaneal structures of the nose, including lateral cartilage, septum cartilage, greater alar cartilage, lesser alar cartilage, sesamoid cartilage, nasal bone, epidermis, adipose tissue, frontal process of the maxilla and fibrofatty tissue.

FIG. 2I shows a medial dissection of a nose, approximately several millimeters from the midsagittal plane, amongst other things showing the septum cartilage and medial crus of greater alar cartilage.

FIG. 2J shows a front view of the bones of a skull including the frontal, nasal and zygomatic bones. Nasal concha are indicated, as are the maxilla, and mandible.

FIG. 2K shows a lateral view of a skull with the outline of the surface of a head, as well as several muscles. The following bones are shown: frontal, sphenoid, nasal, zygomatic, maxilla, mandible, parietal, temporal and occipital. The mental protuberance is indicated. The following muscles are shown: digastricus, masseter, sternocleidomastoideod trapezius.

FIG. 2L shows an anterolateral view of a nose.

### 4.3 Patient Interface

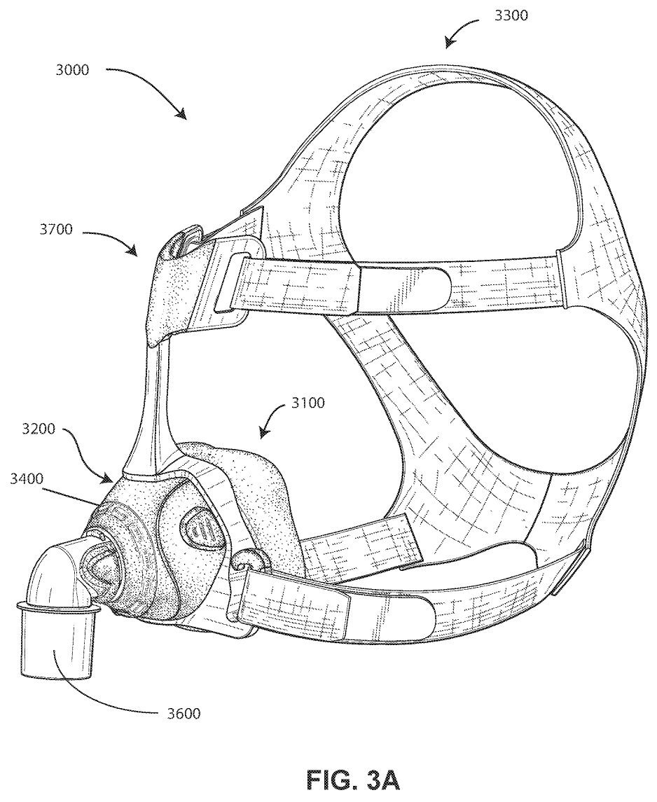

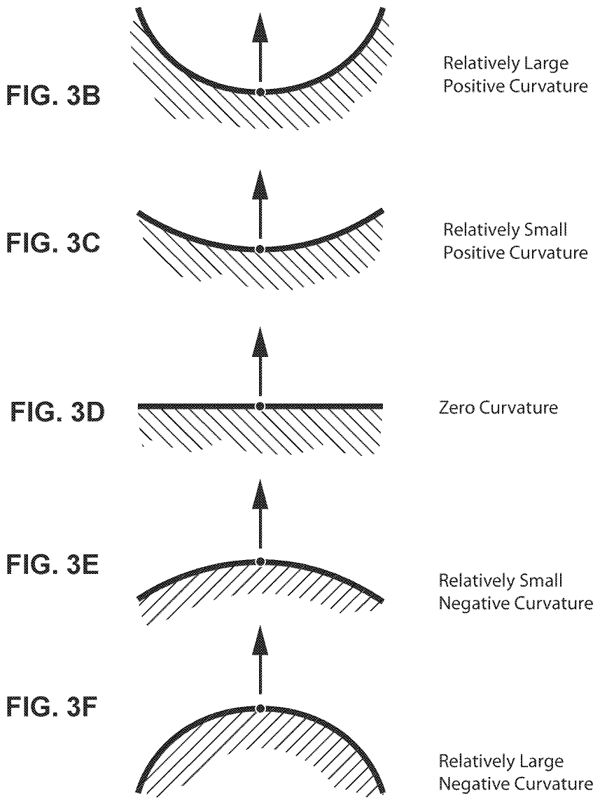

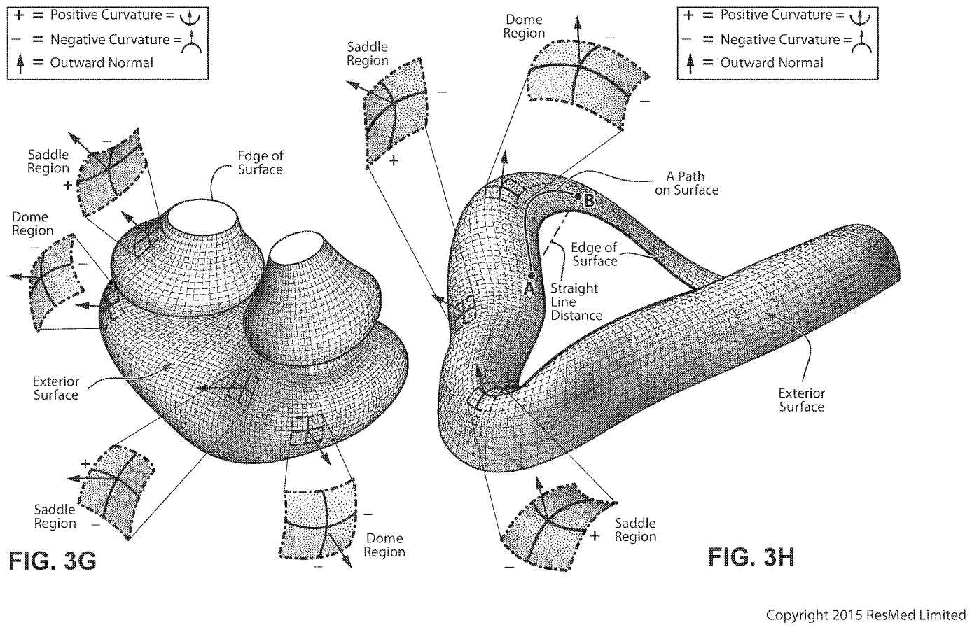

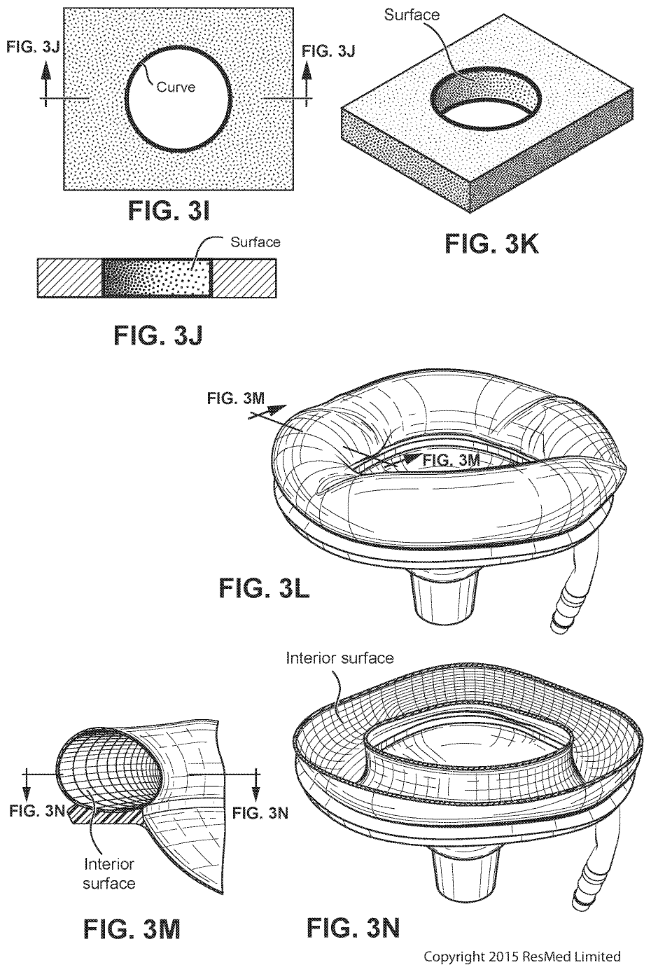

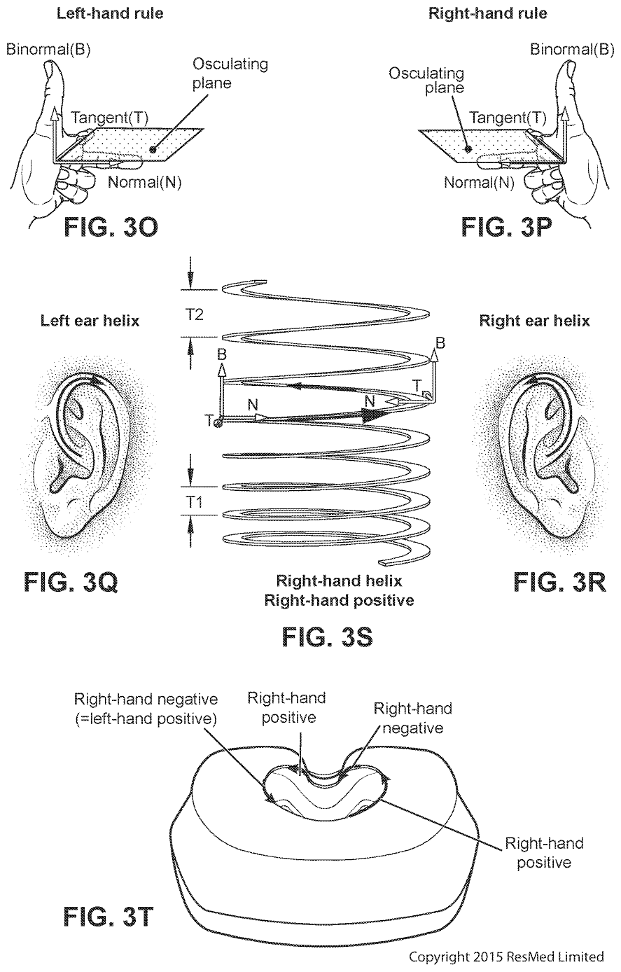

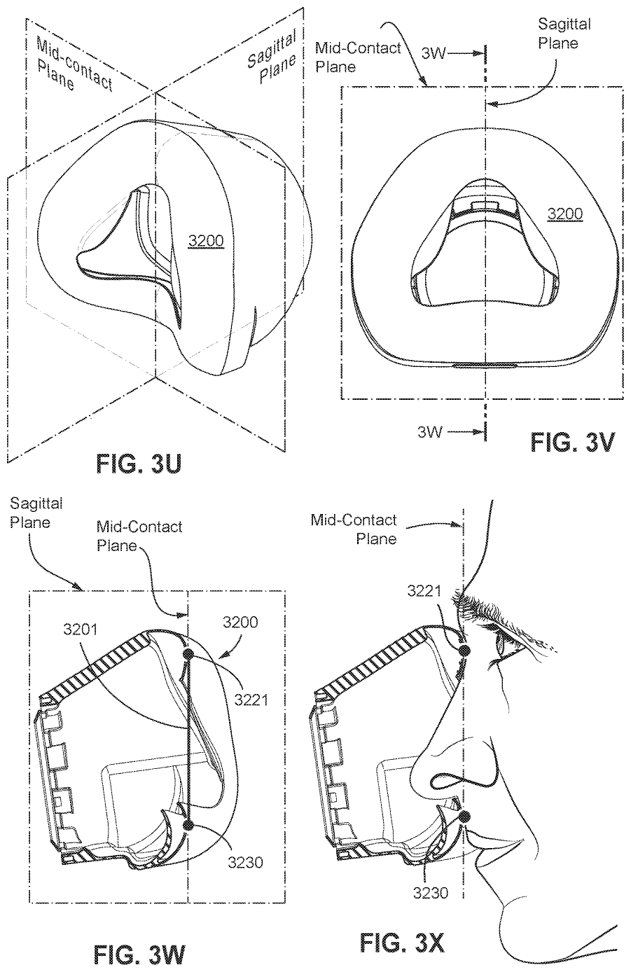

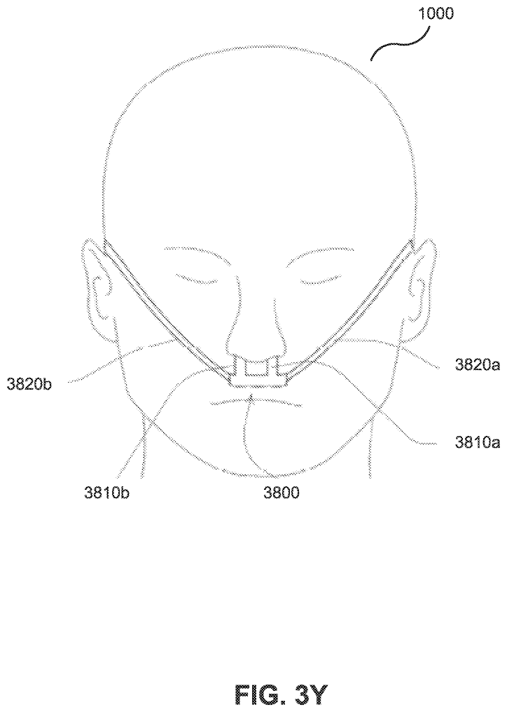

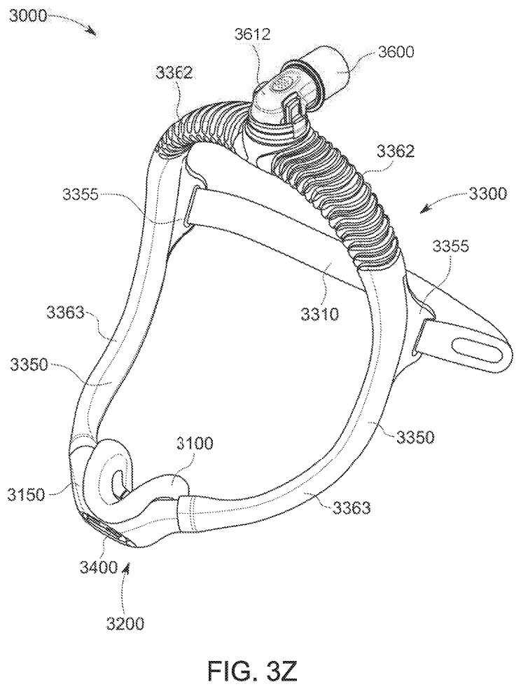

FIG. 3A shows a patient interface in the form of a nasal mask in accordance with one form of the present technology.

FIG. 3B shows a schematic of a cross-section through a structure at a point. An outward normal at the point is indicated. The curvature at the point has a positive sign, and a relatively large magnitude when compared to the magnitude of the curvature shown in FIG. 3C.

FIG. 3C shows a schematic of a cross-section through a structure at a point. An outward normal at the point is indicated. The curvature at the point has a positive sign, and a relatively small magnitude when compared to the magnitude of the curvature shown in FIG. 3B.

FIG. 3D shows a schematic of a cross-section through a structure at a point. An outward normal at the point is indicated. The curvature at the point has a value of zero.

FIG. 3E shows a schematic of a cross-section through a structure at a point. An outward normal at the point is indicated. The curvature at the point has a negative sign, and a relatively small magnitude when compared to the magnitude of the curvature shown in FIG. 3F.

FIG. 3F shows a schematic of a cross-section through a structure at a point. An outward normal at the point is indicated. The curvature at the point has a negative sign, and a relatively large magnitude when compared to the magnitude of the curvature shown in FIG. 3E.

FIG. 3G shows a cushion for a mask that includes two pillows. An exterior surface of the cushion is indicated. An edge of the surface is indicated. Dome and saddle regions are indicated.

FIG. 3H shows a cushion for a mask. An exterior surface of the cushion is indicated. An edge of the surface is indicated. A path on the surface between points A and B is indicated. A straight line distance between A and B is indicated. Two saddle regions and a dome region are indicated.

FIG. 3I shows the surface of a structure, with a one dimensional hole in the surface. The illustrated plane curve forms the boundary of a one dimensional hole.

FIG. 3J shows a cross-section through the structure of FIG. 3I. The illustrated surface bounds a two dimensional hole in the structure of FIG. 3I.

FIG. 3K shows a perspective view of the structure of FIG. 3I, including the two dimensional hole and the one dimensional hole. Also shown is the surface that bounds a two dimensional hole in the structure of FIG. 3I.

FIG. 3L shows a mask having an inflatable bladder as a cushion.

FIG. 3M shows a cross-section through the mask of FIG. 3L, and shows the interior surface of the bladder. The interior surface bounds the two dimensional hole in the mask.

FIG. 3N shows a further cross-section through the mask of FIG. 3L. The interior surface is also indicated.

FIG. 3O illustrates a left-hand rule.

FIG. 3P illustrates a right-hand rule.

FIG. 3Q shows a left ear, including the left ear helix.

FIG. 3R shows a right ear, including the right ear helix.

FIG. 3S shows a right-hand helix.

FIG. 3T shows a view of a mask, including the sign of the torsion of the space curve defined by the edge of the sealing membrane in different regions of the mask.

FIG. 3U shows a view of a plenum chamber 3200 showing a sagittal plane and a mid-contact plane.

FIG. 3V shows a view of a posterior of the plenum chamber of FIG. 3U. The direction of the view is normal to the mid-contact plane. The sagittal plane in FIG. 3V bisects the plenum chamber into left-hand and right-hand sides.

FIG. 3W shows a cross-section through the plenum chamber of FIG. 3V, the cross-section being taken at the sagittal plane shown in FIG. 3V. A ‘mid-contact’ plane is shown. The mid-contact plane is perpendicular to the sagittal plane. The orientation of the mid-contact plane corresponds to the orientation of a chord 3201 which lies on the sagittal plane and just touches the cushion of the plenum chamber at two points on the sagittal plane: a superior point 3221 and an inferior point 3230. Depending on the geometry of the cushion in this region, the mid-contact plane may be a tangent at both the superior and inferior points.

FIG. 3X shows the plenum chamber 3200 of FIG. 3U in position for use on a face. The sagittal plane of the plenum chamber 3200 generally coincides with the midsagittal plane of the face when the plenum chamber is in position for use. The mid-contact plane corresponds generally to the ‘plane of the face’ when the plenum chamber is in position for use. In FIG. 3X the plenum chamber 3200 is that of a nasal mask, and the superior point 3221 sits approximately on the sellion, while the inferior point 3230 sits on the lip superior.

FIG. 3Y shows a patient interface in the form of a nasal cannula in accordance with one form of the present technology.

FIG. 3Z shows a patient interface having conduit headgear in accordance with one form of the present technology.

### 4.4 RPT Device

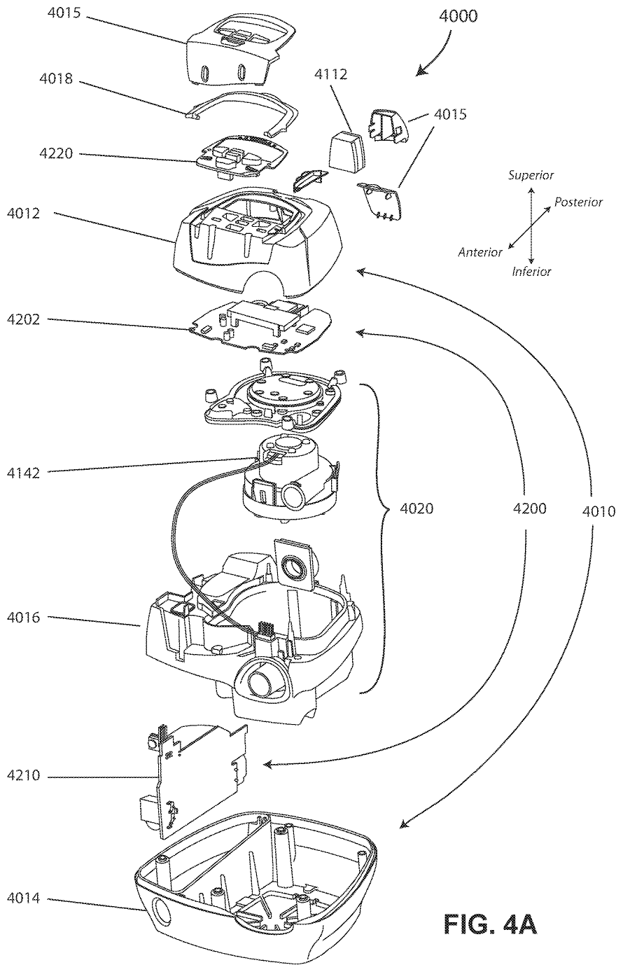

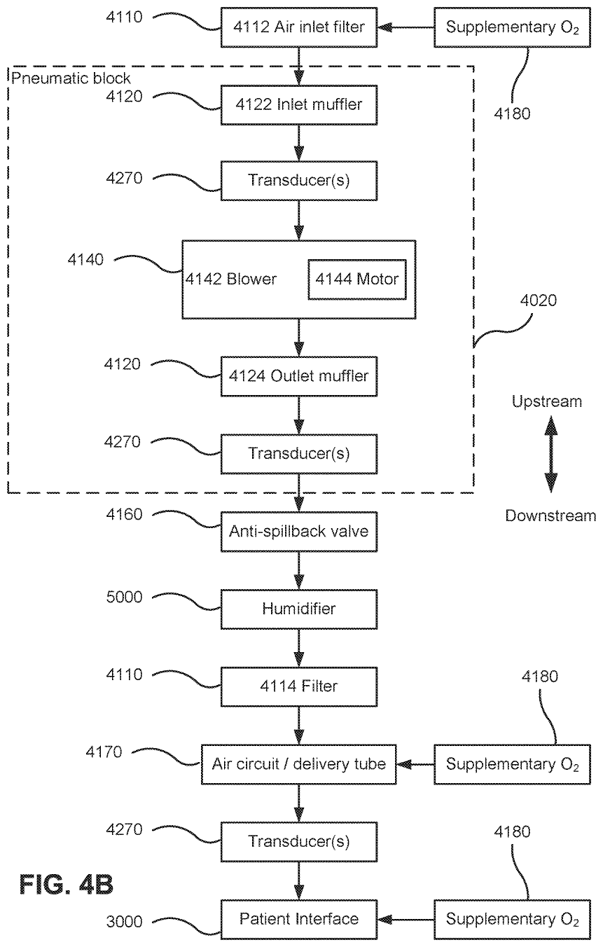

FIG. 4A shows an RPT device in accordance with one form of the present technology.

FIG. 4B is a schematic diagram of the pneumatic path of an RPT device in accordance with one form of the present technology. The directions of upstream and downstream are indicated with reference to the blower and the patient interface. The blower is defined to be upstream of the patient interface and the patient interface is defined to be downstream of the blower, regardless of the actual flow direction at any particular moment. Items which are located within the pneumatic path between the blower and the patient interface are downstream of the blower and upstream of the patient interface.

### 4.5 Humidifier

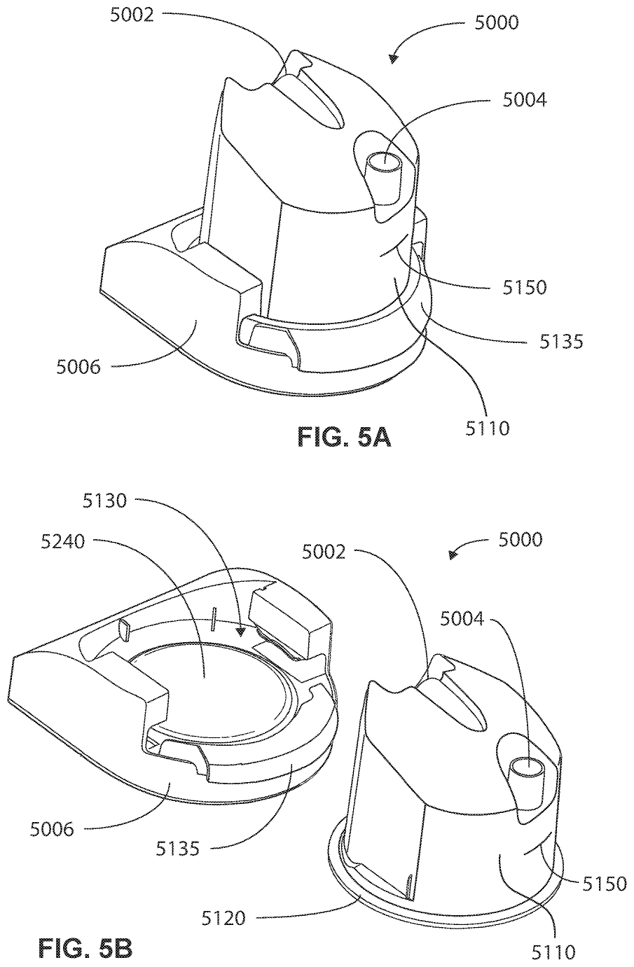

FIG. 5A shows an isometric view of a humidifier in accordance with one form of the present technology.

FIG. 5B shows an isometric view of a humidifier in accordance with one form of the present technology, showing a humidifier reservoir 5110 removed from the humidifier reservoir dock 5130.

### 4.6 Breathing Waveforms

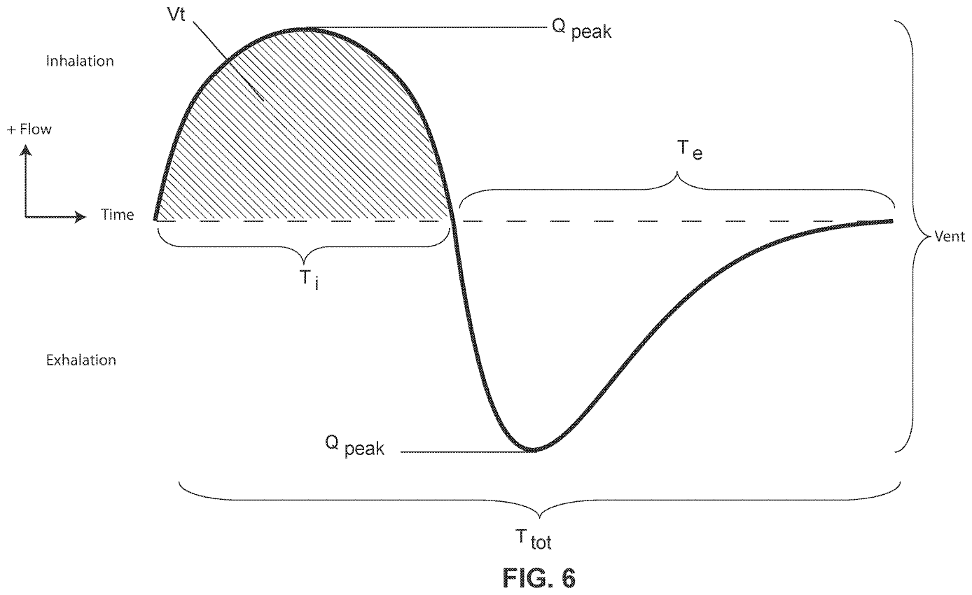

FIG. 6A shows a model typical breath waveform of a person while sleeping.

### 4.7 Patient Interfaces According to Futher Examples of the Present Technology

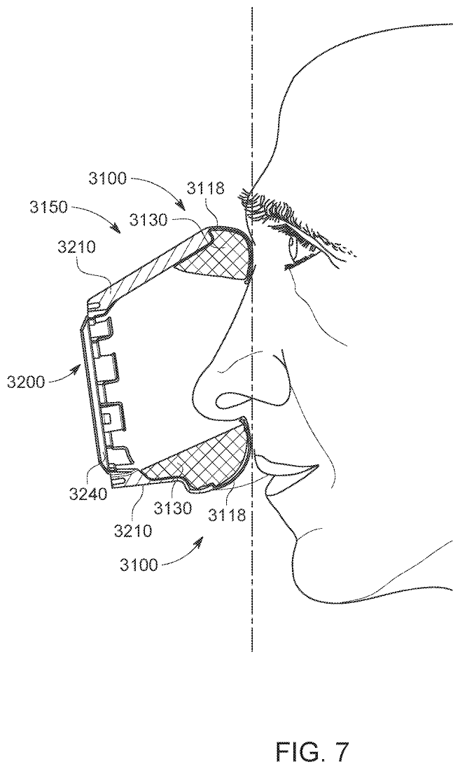

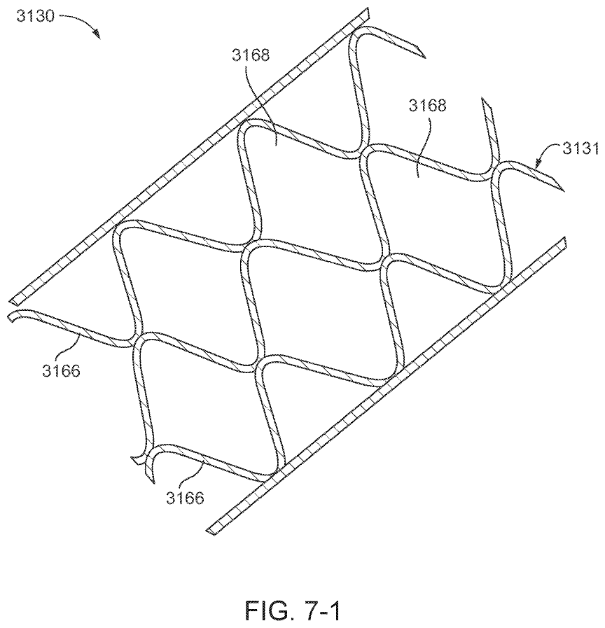

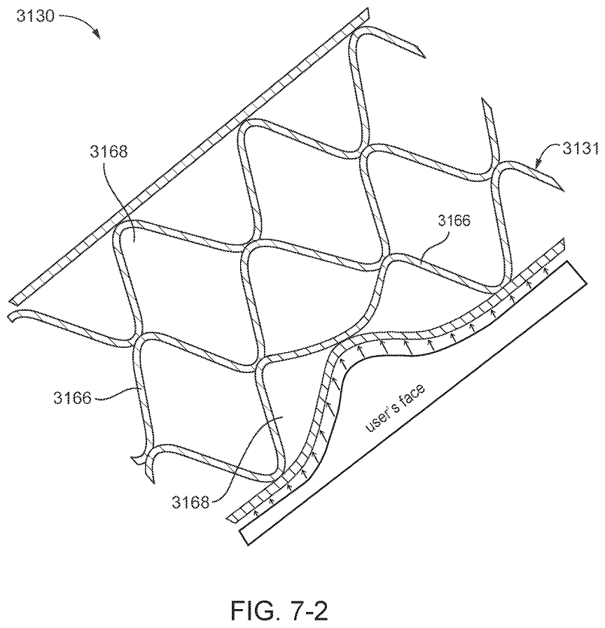

FIG. 7 is a cross section view of a cushion module of a patient interface according to an example of the present technology in an in-use position.

FIG. 7-1 is a detail view of a portion of a cushion of a patient interface according to an example of the present technology.

FIG. 7-2 is a detail view of a portion of the cushion of FIG. 7-1 when in use.

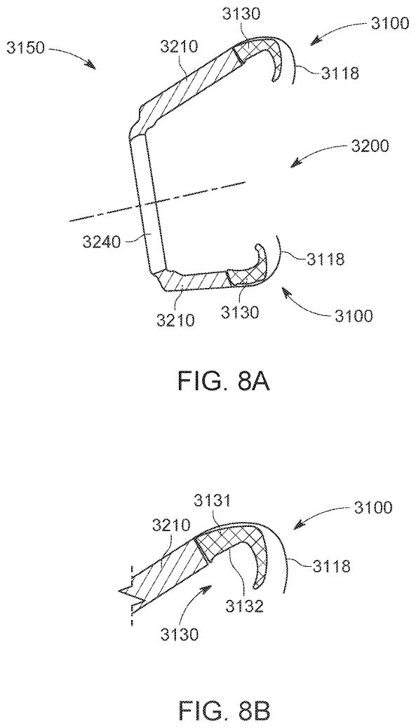

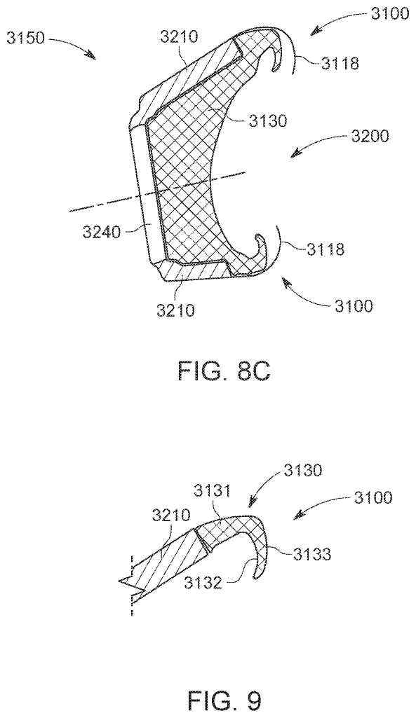

FIG. 8A is a cross section view of a cushion module of a patient interface according to another example of the present technology.

FIG. 8B is a detail view of a portion of the patient interface of FIG. 8A.

FIG. 8C is a cross section view of a portion of a patient interface according to another example of the present technology.

FIG. 9 is a detail view of a portion of a patient interface according to another example of the present technology.

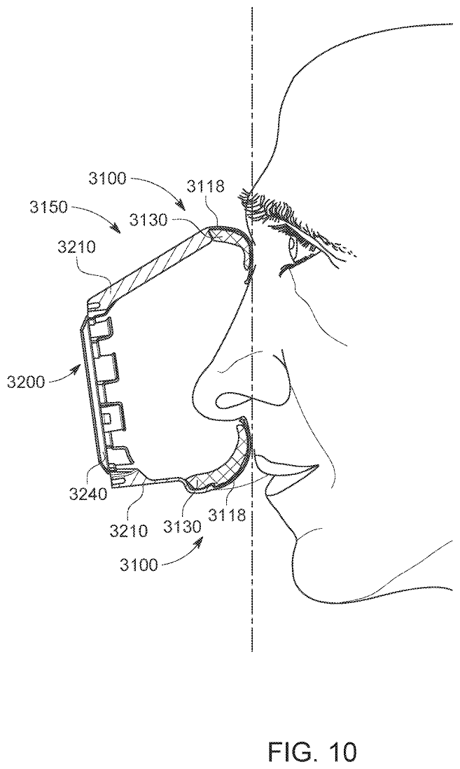

FIG. 10 is a cross section view of a cushion module of a patient interface according to another example of the present technology in an in-use position.

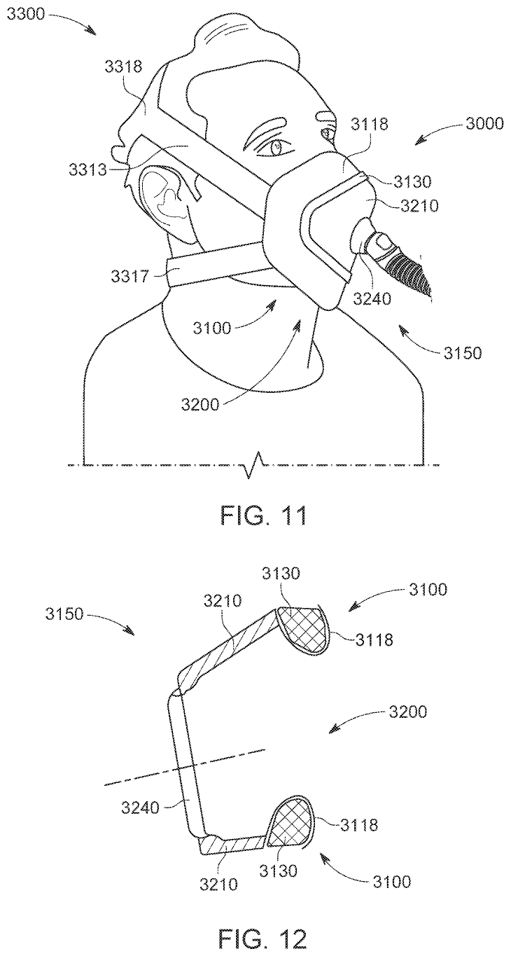

FIG. 11 is a perspective view of a patient interface according to another example of the present technology.

FIG. 12 is a cross section view of a cushion module of a patient interface according to another example of the present technology.

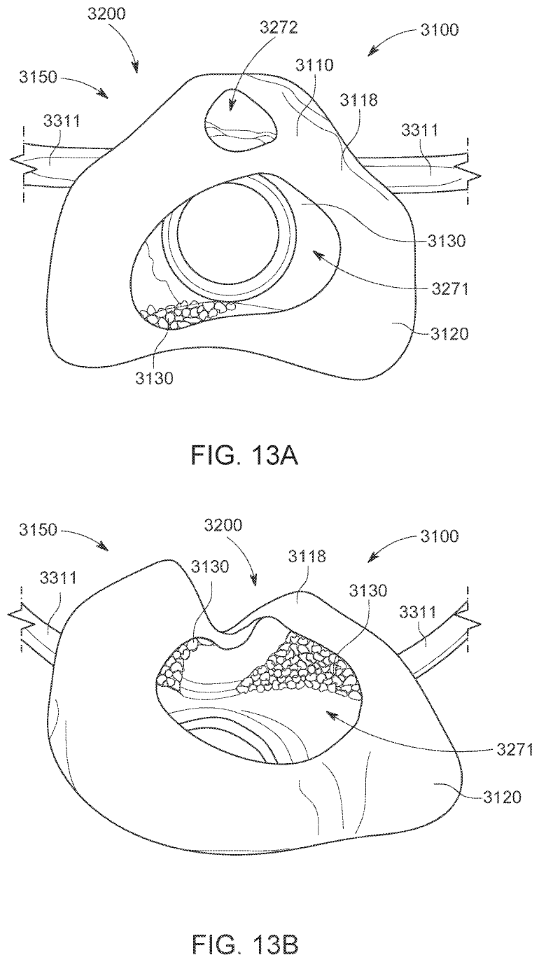

FIG. 13A is a posterior view of a cushion module of a patient interface according to another example of the present technology.

FIG. 13B is a posteroinferior perspective view of the cushion module of FIG. 13A.

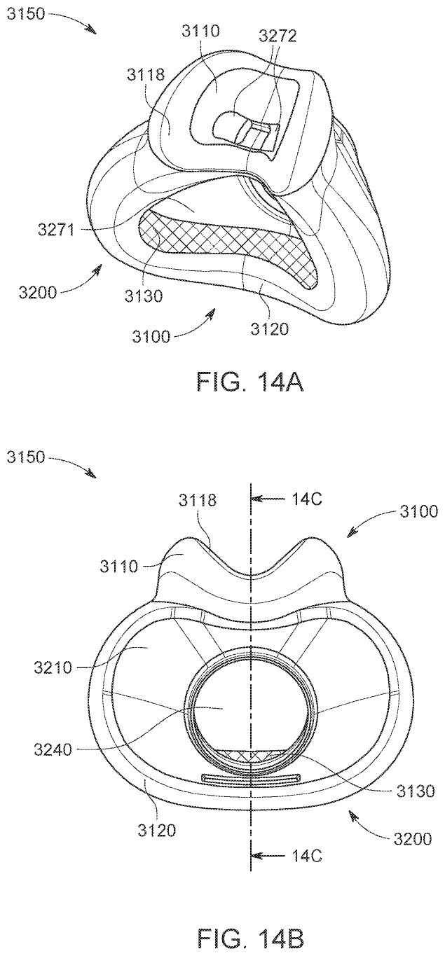

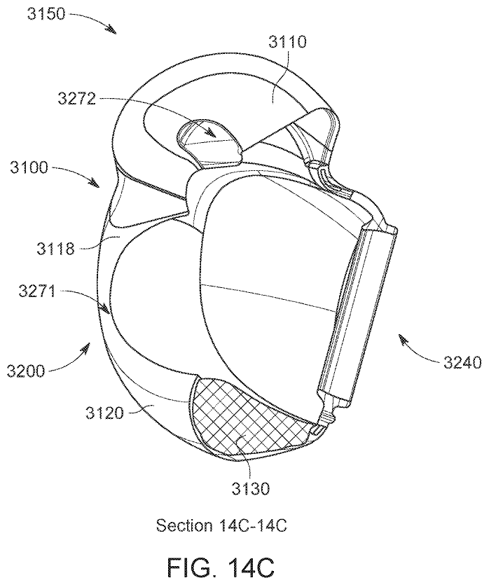

FIG. 14A is a posteriosuperior perspective view of a cushion module of a patient interface according to another example of the present technology.

FIG. 14B is an anterior view of the cushion module of FIG. 14A.

FIG. 14C is a cross section view of the cushion module of FIG. 14A at section 14C-14C identified in FIG. 14B.

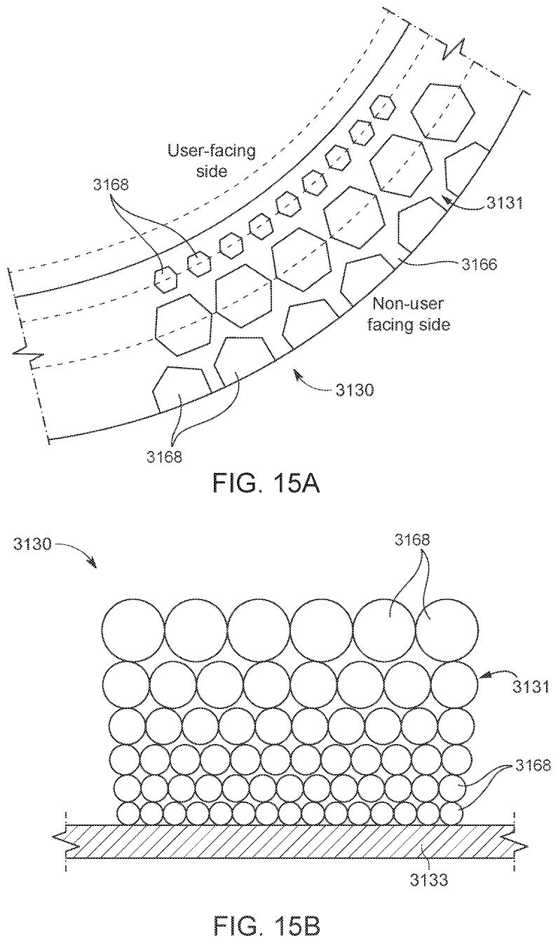

FIG. 15A is a detail view of a portion of a cushion of a patient interface according to another example of the present technology.

FIG. 15B is a schematic view of a portion of a cushion of a patient interface according to another example of the present technology.

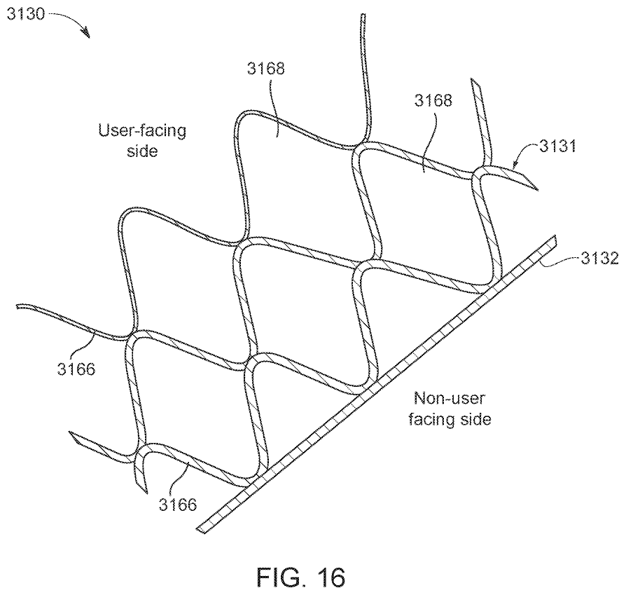

FIG. 16 is a detail view of a portion of a cushion of a patient interface according to another example of the present technology.

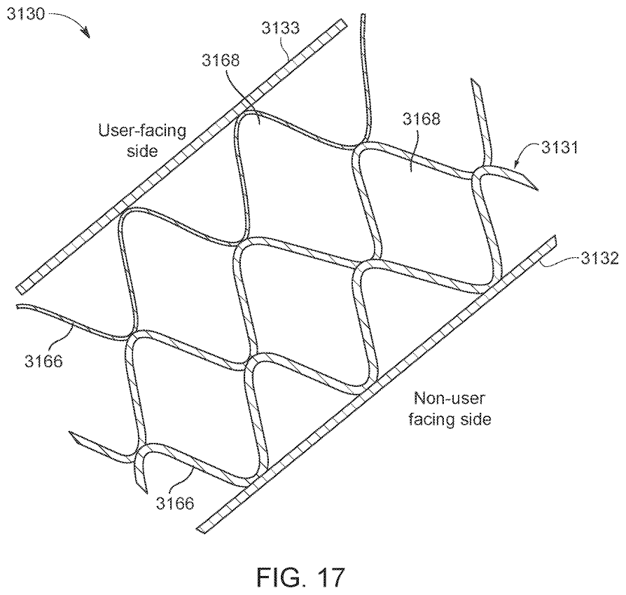

FIG. 17 is a detail view of a portion of a cushion of a patient interface according to another example of the present technology.

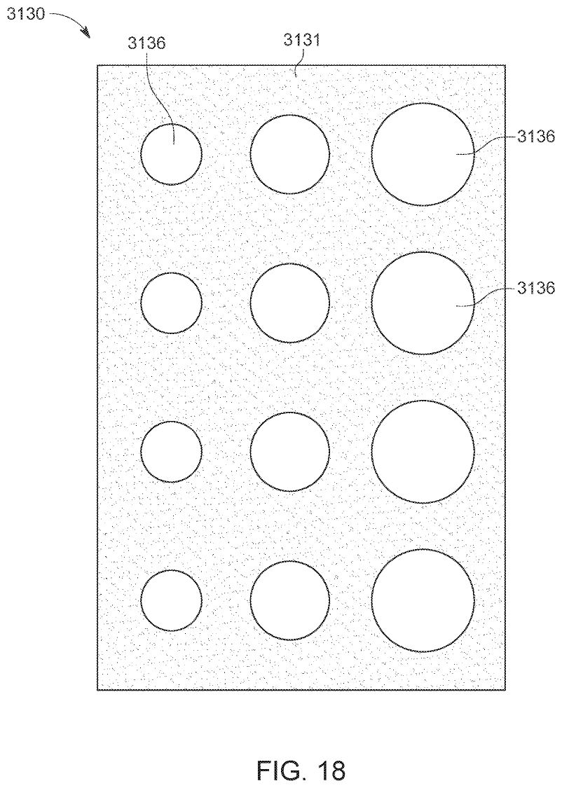

FIG. 18 is a schematic view of a cushion of a patient interface according to another example of the present technology.

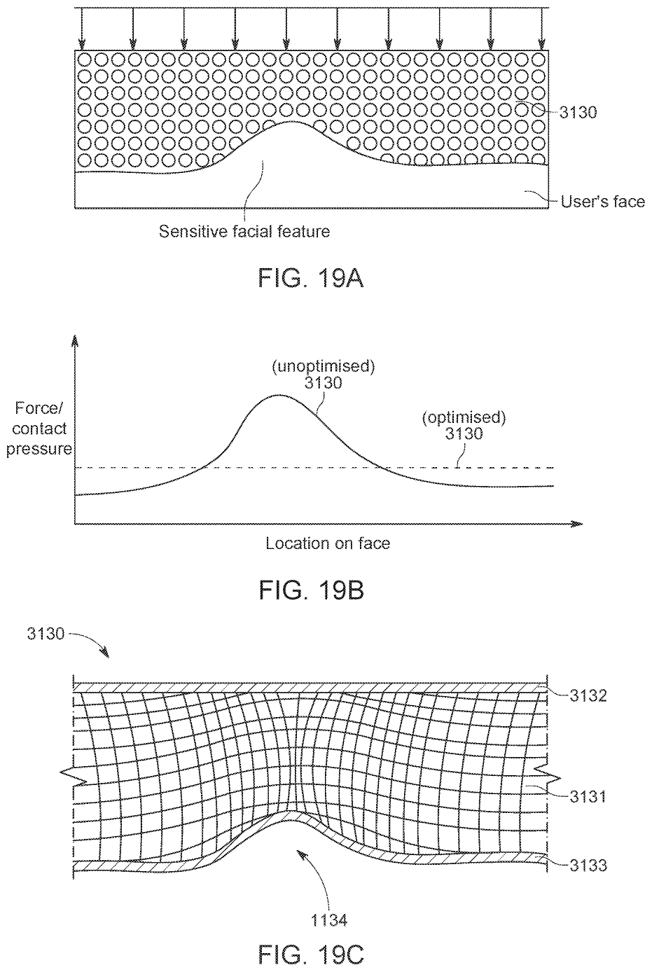

FIG. 19A is a schematic view of a cushion of a patient interface according to another example of the present technology in contact with a user's face, subjected to loading.

FIG. 19B is a plot of force/contact pressure over the user's face for cushions according to examples of the present technology, when subjected to loading as shown in FIG. 19A.

FIG. 19C is a schematic view of a portion of a cushion of a patient interface according to another example of the present technology.

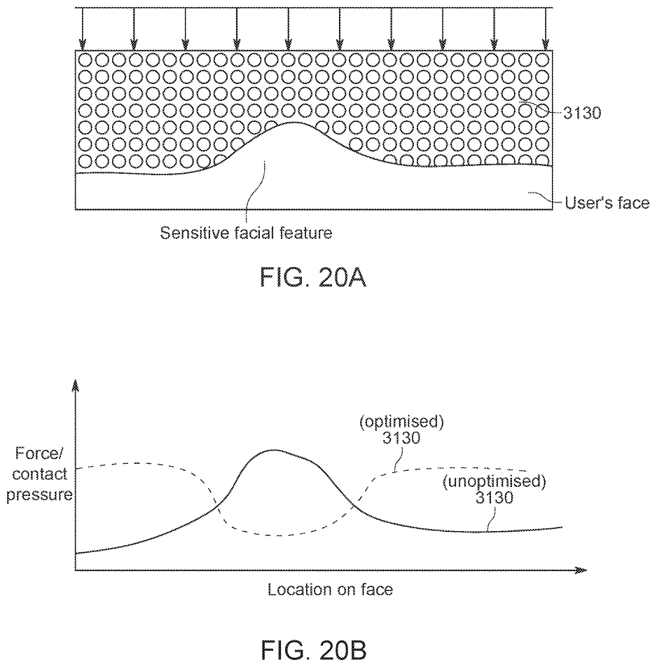

FIG. 20A is a schematic view of a cushion of a patient interface according to another example of the present technology in contact with a user's face, subjected to loading.

FIG. 20B is a plot of force/contact pressure over the user's face for cushions according to examples of the present technology, when subjected to loading as shown in FIG. 20A.

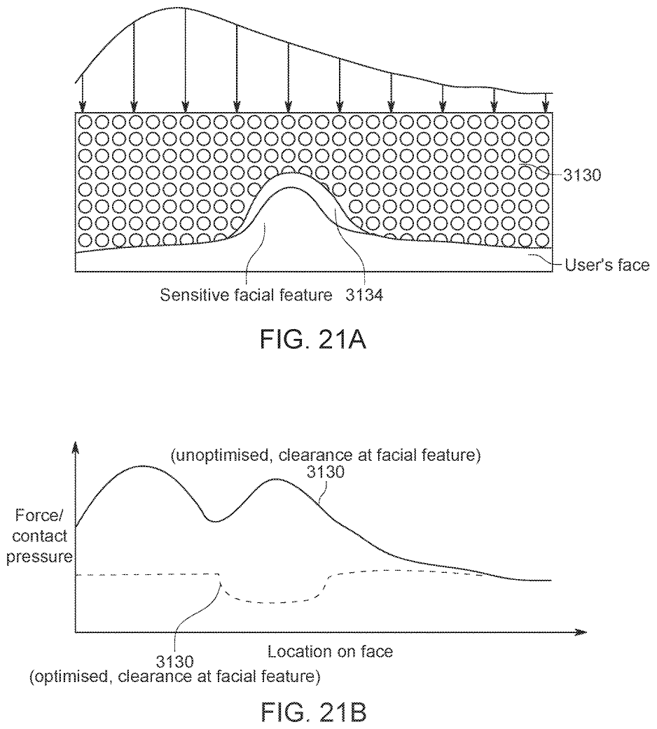

FIG. 21A is a schematic view of a cushion of a patient interface according to another example of the present technology in contact with a user's face, subjected to loading.

FIG. 21B is a plot of force/contact pressure over the user's face for cushions according to examples of the present technology, when subjected to loading as shown in FIG. 21A.

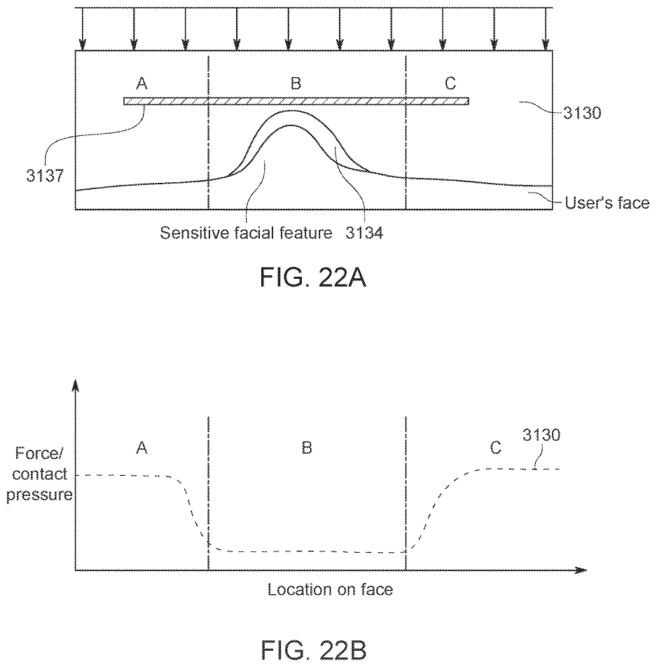

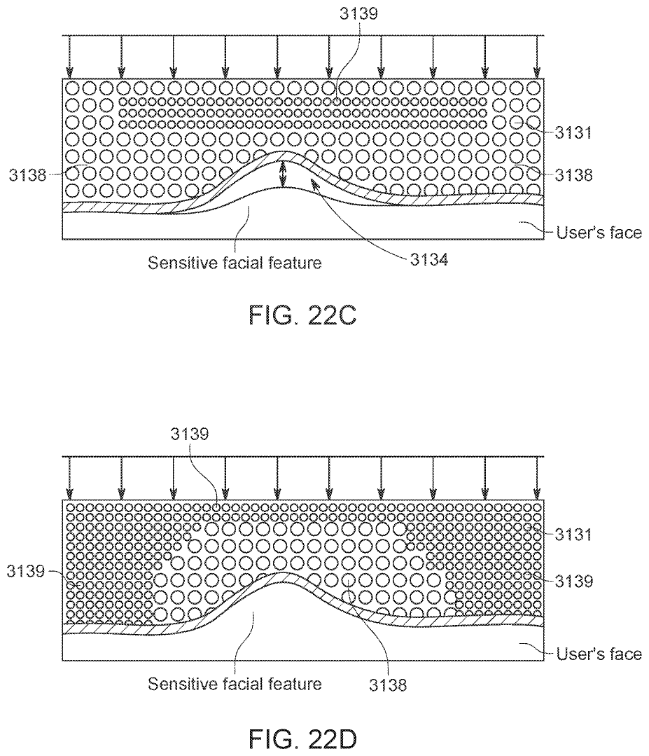

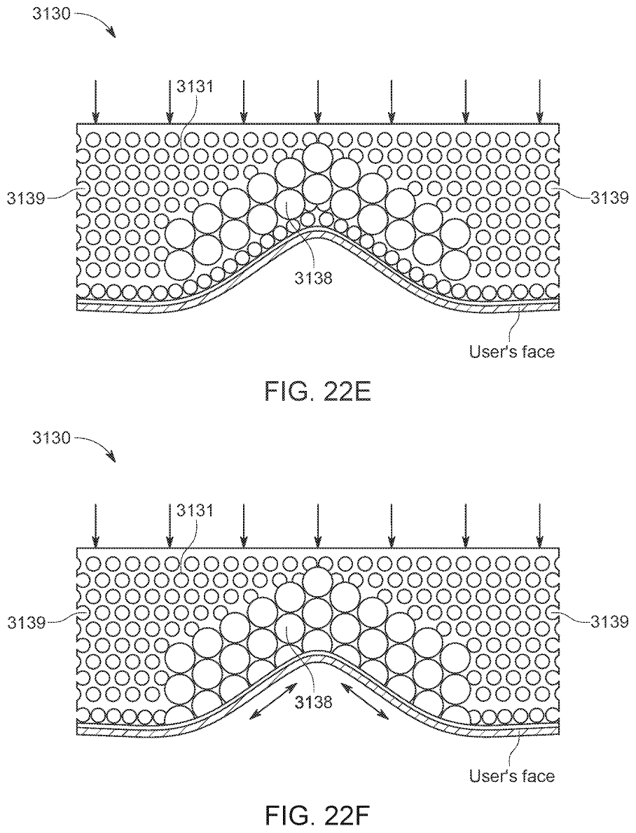

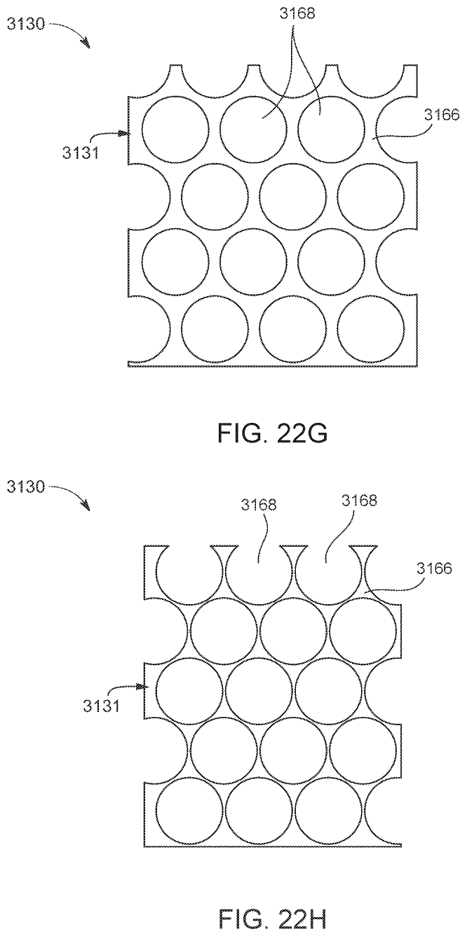

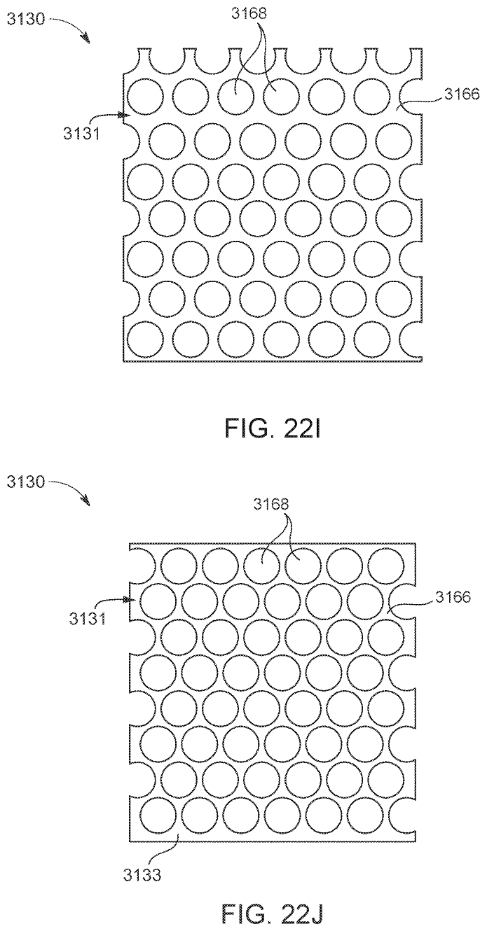

FIG. 22A is a schematic view of a cushion of a patient interface according to another example of the present technology in contact with a user's face, subjected to loading.

FIG. 22B is a plot of force/contact pressure over the user's face for the cushion of FIG. 22A when subjected to loading as shown in FIG. 22A.

FIG. 22C is a schematic view of a cushion of a patient interface according to another example of the present technology in contact with a user's face, subjected to loading.

FIG. 22D is a schematic view of a cushion of a patient interface according to another example of the present technology in contact with a user's face, subjected to loading.

FIG. 22E is a schematic view of a cushion of a patient interface according to another example of the present technology in contact with a user's face, subjected to loading.

FIG. 22F is a schematic view of a cushion of a patient interface according to another example of the present technology in contact with a user's face, subjected to loading.

FIG. 22G is a schematic view of a cushion having a cushion body comprising a lattice structure according to another example of the present technology.

FIG. 22H is a schematic view of a cushion having a cushion body comprising a lattice structure according to another example of the present technology.

FIG. 22I is a schematic view of a cushion having a cushion body comprising a lattice structure according to another example of the present technology.

FIG. 22J is a schematic view of a cushion having a cushion body comprising a lattice structure according to another example of the present technology.

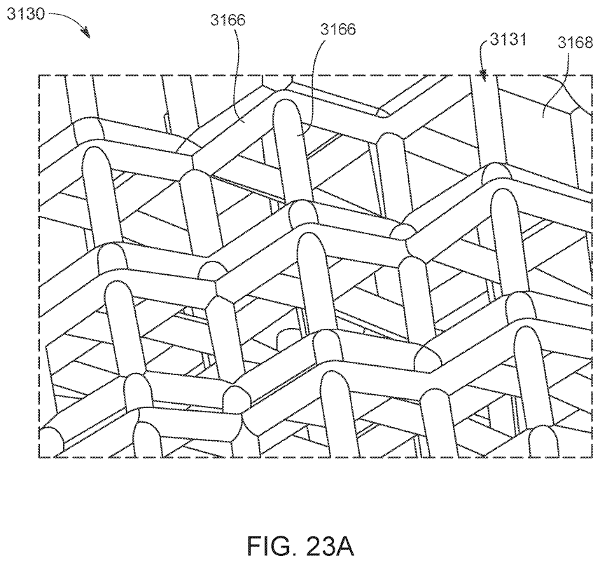

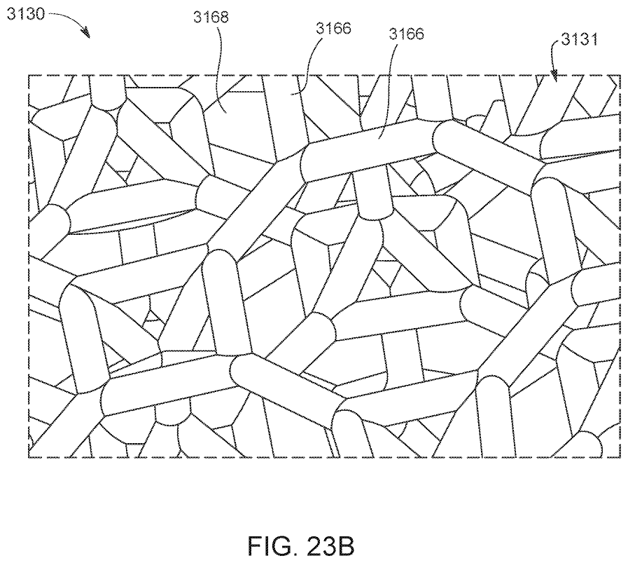

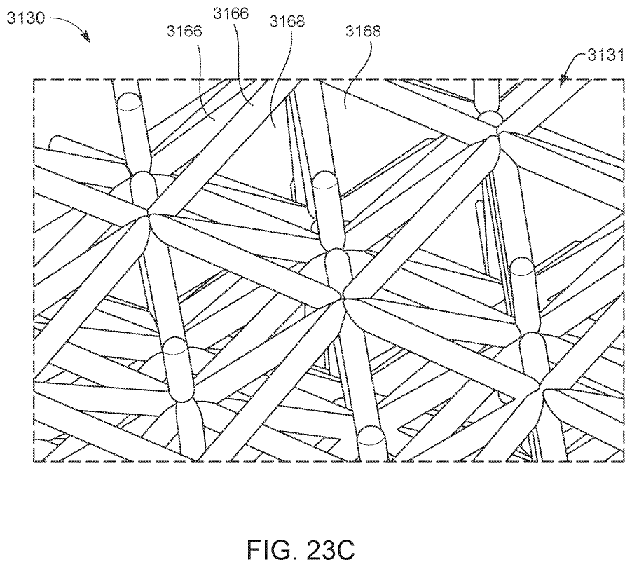

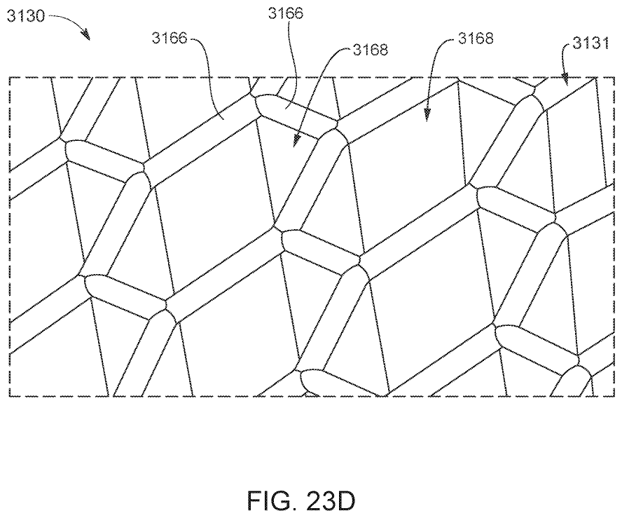

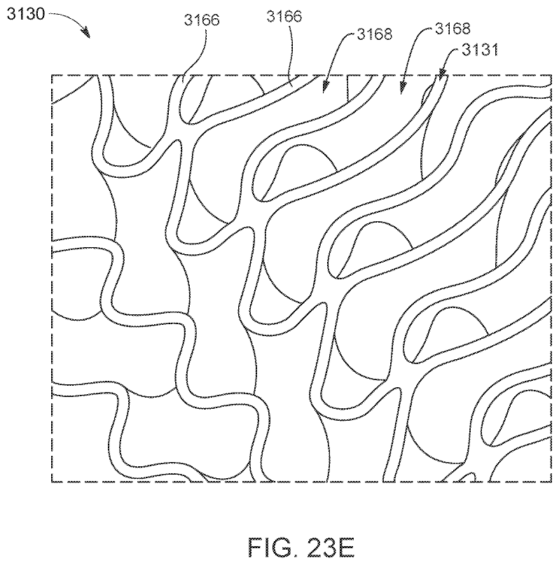

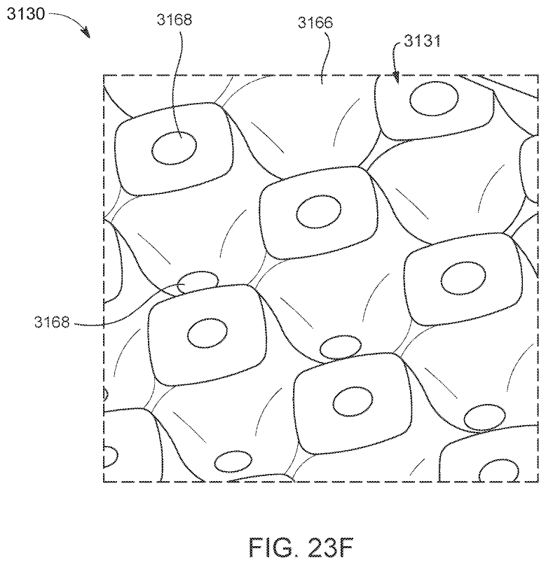

FIGS. 23A-23F are detail views of cushions according to examples of the present technology.

### 4.8 Automatic Sizing

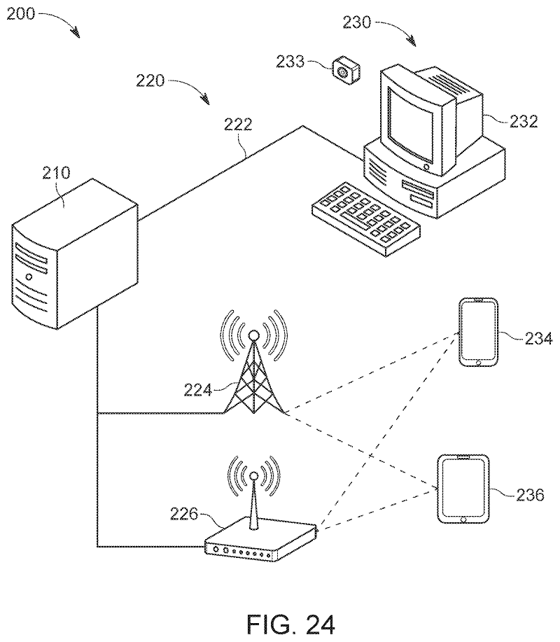

FIG. 24 is a diagram of an example system for automatically sizing a facial interface which includes a computing device.

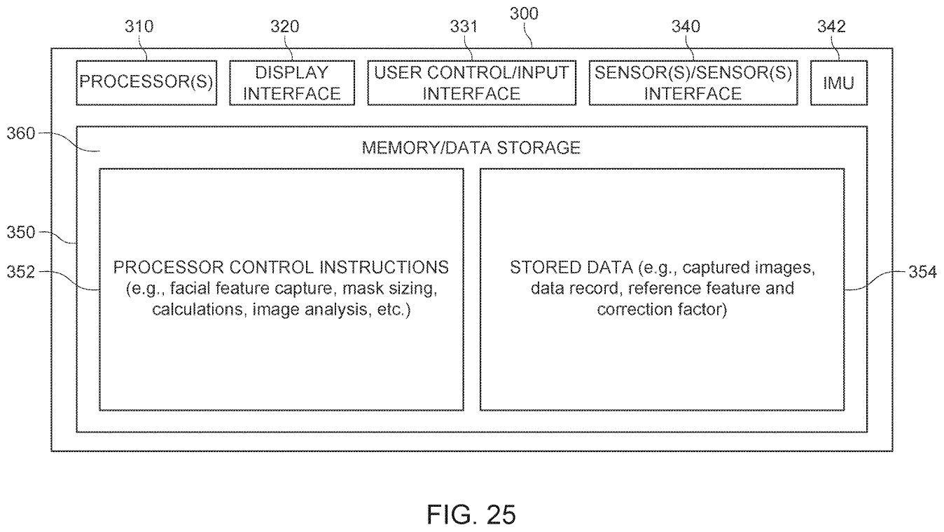

FIG. 25 is a block diagram of an example architecture of a computing device for the system of FIG. 24 including example components suitable for implementing the methodologies of the present technology.

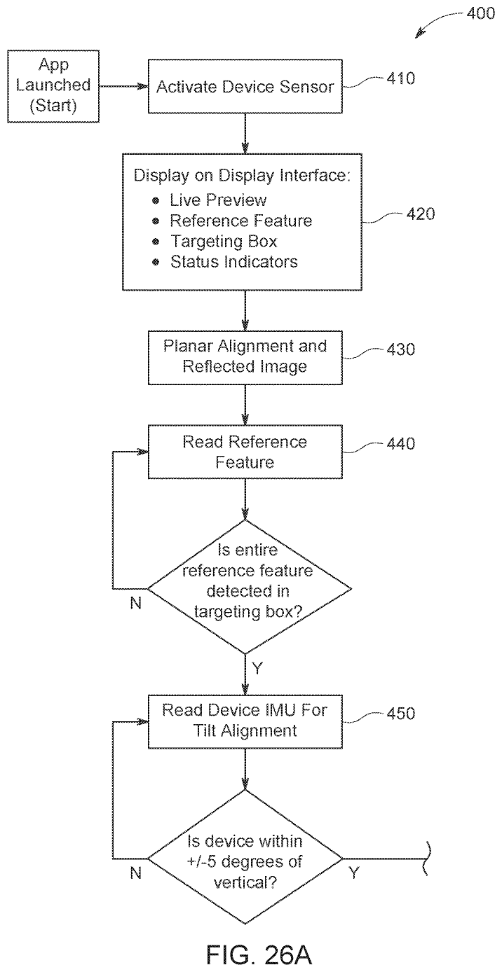

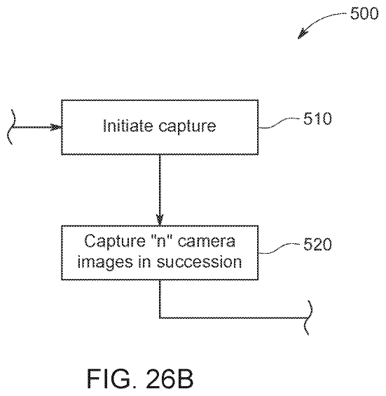

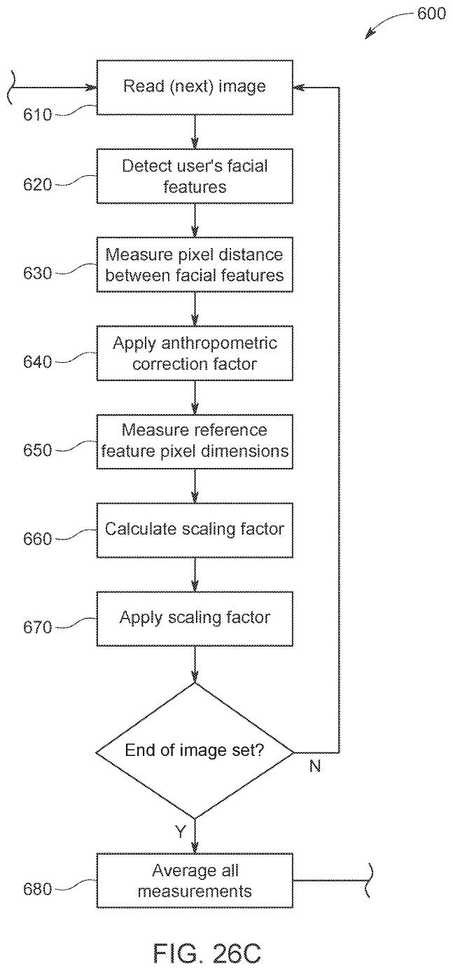

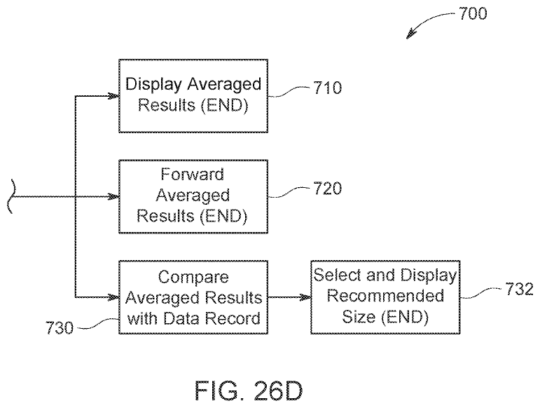

FIG. 26A is a flow diagram of a pre-capture phase method of an example version of the present technology.

FIG. 26B is a flow diagram of a capture phase method of some versions of the present technology.

FIG. 26C is a flow diagram of a post-capture image processing phase method of some versions of the present technology.

FIG. 26D is a flow diagram of a comparison and output phase method of some versions of an exemplary method embodiment of the present technology.

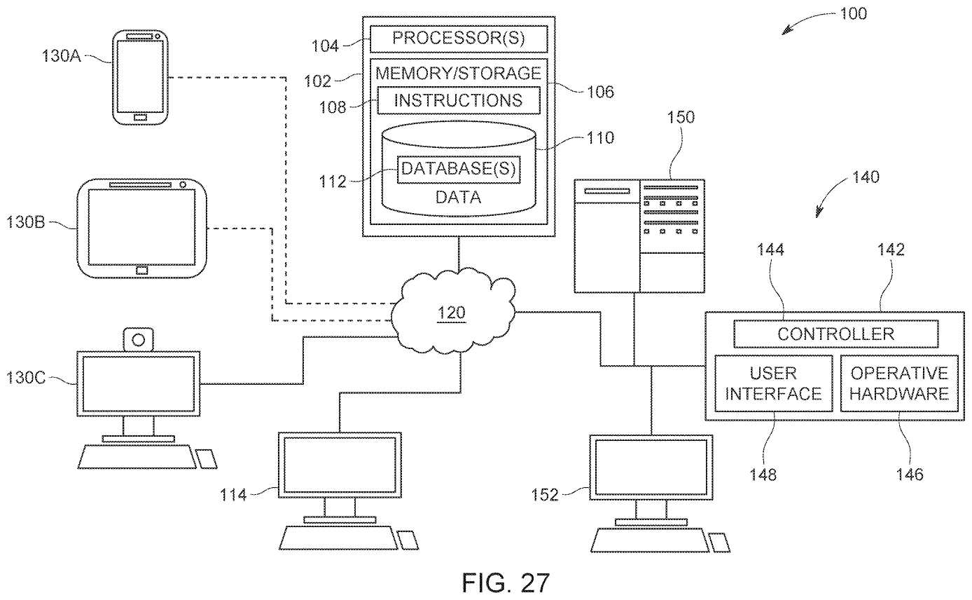

FIG. 27 shows a schematic view of a system 100 according to another example of the present technology.

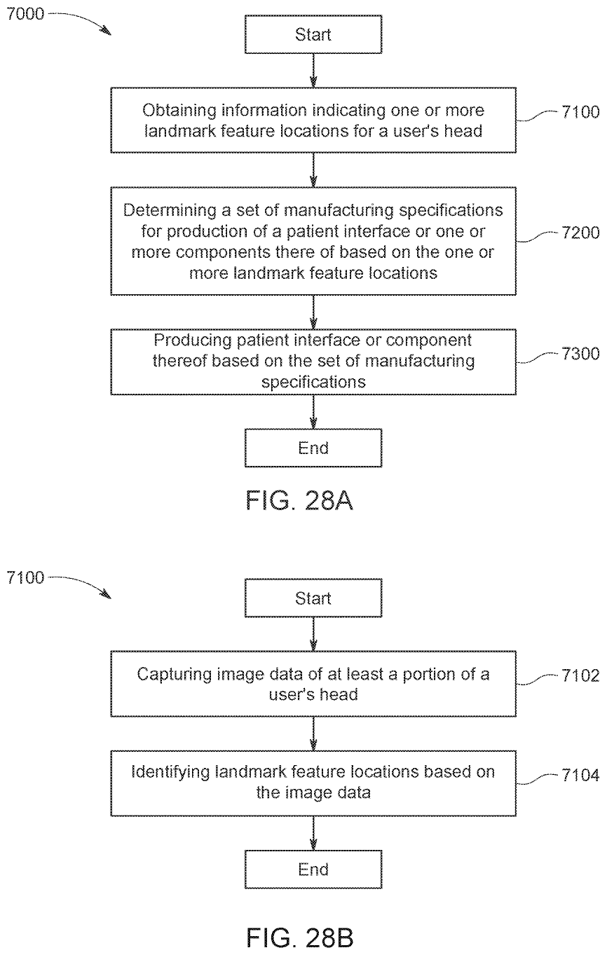

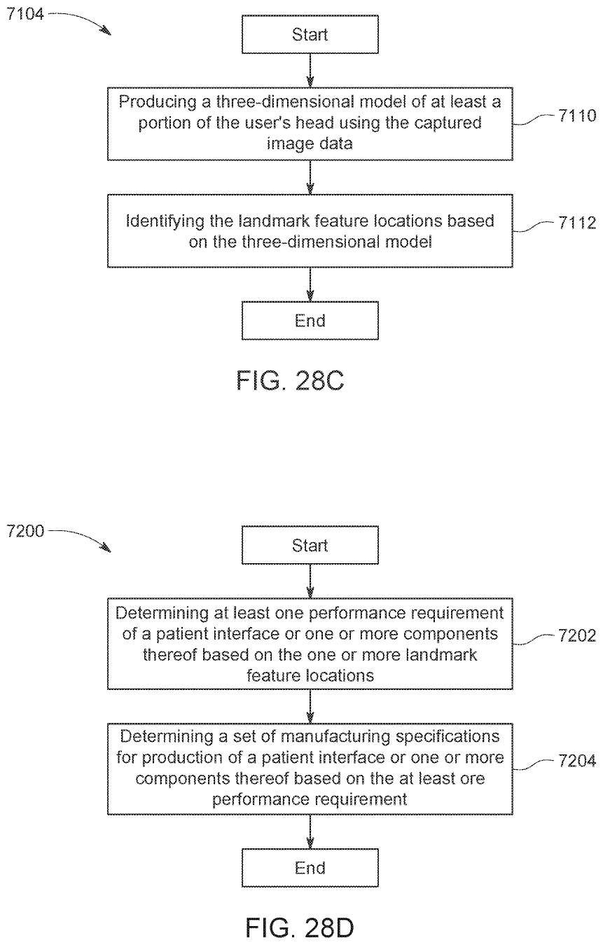

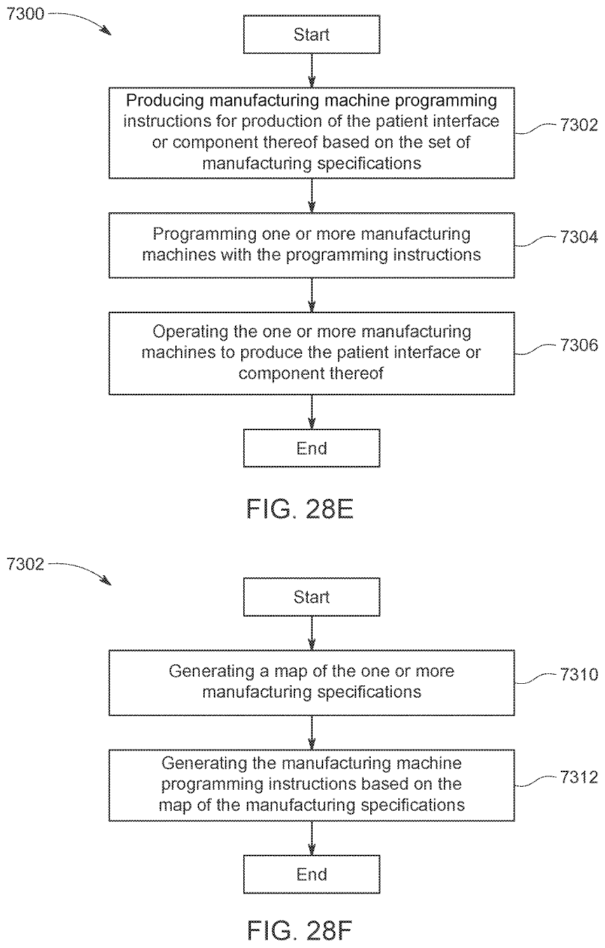

FIG. 28A to 28F show flow charts of a method 7000 and aspects thereof according to another example of the present technology.

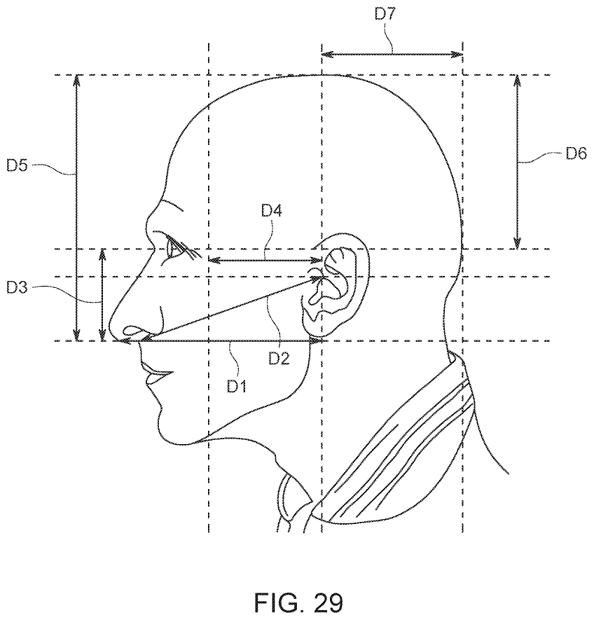

FIG. 29 shows a side view of a user's head having a number of distances identified, relevant to the method 7000.

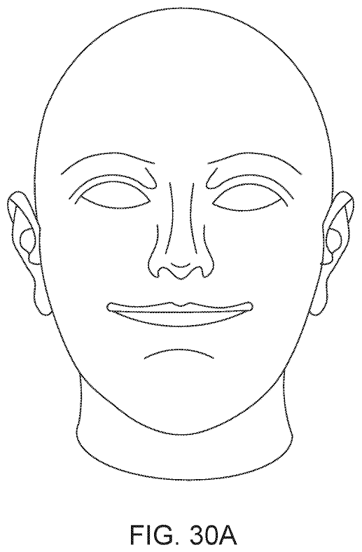

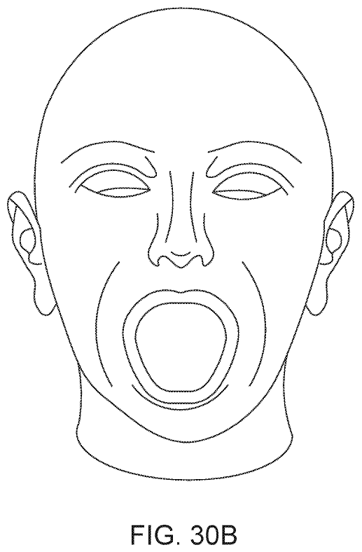

FIGS. 30A and 30B depict facial expressions of a patient which may be captured as part of the method 7000.

### 5 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### 5.1 Therapy

In one form, the present technology comprises a method for treating a respiratory disorder comprising applying positive pressure to the entrance of the airways of a patient 1000.

In certain examples of the present technology, a supply of air at positive pressure is provided to the nasal passages of the patient via one or both nares.

In certain examples of the present technology, mouth breathing is limited, restricted or prevented.

### 5.2 Respiratory Therapy Systems

In one form, the present technology comprises a respiratory therapy system for treating a respiratory disorder. The respiratory therapy system may comprise an RPT device 4000 for supplying a flow of air to the patient 1000 via an air circuit 4170 and a patient interface 3000 or 3800.

### 5.3 Patient Interface

A non-invasive patient interface 3000, such as that shown in FIG. 3A, in accordance with one aspect of the present technology comprises the following functional aspects: a seal-forming structure 3100, a plenum chamber 3200, a positioning and stabilising structure 3300, a vent 3400, one form of connection port 3600 for connection to air circuit 4170, and a forehead support. In some forms a functional aspect may be provided by one or more physical components. In some forms, one physical component may provide one or more functional aspects. In use the seal-forming structure 3100 is arranged to surround an entrance to the airways of the patient so as to maintain positive pressure at the entrance(s) to the airways of the patient 1000. The sealed patient interface 3000 is therefore suitable for delivery of positive pressure therapy.

As shown in FIG. 3Z, a non-invasive patient interface 3000 in accordance with another aspect of the present technology comprises the following functional aspects: a seal-forming structure 3100, a plenum chamber 3200, a positioning and stabilising structure 3300, a vent 3400 and one form of connection port 3600 for connection to an air circuit (such as the air circuit 4170 shown in FIGS. 1A-1C). The plenum chamber 3200 may be formed of one or more modular components in the sense that it or they can be replaced with different components, for example components of a different size.

If a patient interface is unable to comfortably deliver a minimum level of positive pressure to the airways, the patient interface may be unsuitable for respiratory pressure therapy.

The patient interface 3000 in accordance with one form of the present technology is constructed and arranged to be able to provide a supply of air at a positive pressure of at least 6 cmH2 O with respect to ambient.

The patient interface 3000 in accordance with one form of the present technology is constructed and arranged to be able to provide a supply of air at a positive pressure of at least 10 cmH2O with respect to ambient.

The patient interface 3000 in accordance with one form of the present technology is constructed and arranged to be able to provide a supply of air at a positive pressure of at least 20 cmH2O with respect to ambient.

### 5.3.1 Seal-Forming Structure

The patient interface 3000 may comprise a seal-forming structure 3100.

The seal-forming structure 3100 may be constructed and arranged to form a seal with a region of the patient's face surrounding an entrance to the patient's airways. Furthermore, the seal-forming structure 3100 may have a hole therein such that the flow of air at said therapeutic pressure is delivered to at least an entrance to the patient's nares in use. The seal-forming structure 3100 may be constructed and arranged to maintain the therapeutic pressure in the plenum chamber 3200 throughout the patient's respiratory cycle in use.

In one form of the present technology, a seal-forming structure 3100 provides a target seal-forming region, and may additionally provide a cushioning function. The target seal-forming region is a region on the seal-forming structure 3100 where sealing may occur. The region where sealing actually occurs-the actual sealing surface-may change within a given treatment session, from day to day, and from patient to patient, depending on a range of factors including for example, where the patient interface was placed on the face, tension in the positioning and stabilising structure and the shape of a patient's face.

In one form the target seal-forming region is located on an outside surface of the seal-forming structure 3100.

In certain forms of the present technology, the seal-forming structure 3100 is constructed from a biocompatible material, e.g. silicone rubber. In other forms the seal-forming structure 3100 comprises a foam undercushion 3110 and a textile membrane portion 3220, as described further below.

A seal-forming structure 3100 in accordance with the present technology may be constructed from a soft, flexible, resilient material such as silicone.

In certain forms of the present technology, a system is provided comprising more than one a seal-forming structure 3100, each being configured to correspond to a different size and/or shape range. For example the system may comprise one form of a seal-forming structure 3100 suitable for a large sized head, but not a small sized head and another suitable for a small sized head, but not a large sized head. However, examples of the technology may be suitable for a large range of heads, and so may be used by patients having a relatively large head and a relatively small head.

### 5.3.1.1 Sealing Mechanisms

In one form, the seal-forming structure includes a sealing flange utilizing a pressure assisted sealing mechanism. In use, the sealing flange can readily respond to a system positive pressure in the interior of the plenum chamber 3200 acting on its underside to urge it into tight sealing engagement with the face. The pressure assisted mechanism may act in conjunction with elastic tension in the positioning and stabilising structure.

In one form, the seal-forming structure 3100 comprises a sealing flange and a support flange. The sealing flange comprises a relatively thin member with a thickness of less than about 1 mm, for example about 0.25 mm to about 0.45 mm, which extends around the perimeter of the plenum chamber 3200. Support flange may be relatively thicker than the sealing flange. The support flange is disposed between the sealing flange and the marginal edge of the plenum chamber 3200, and extends at least part of the way around the perimeter. The support flange is or includes a spring-like element and functions to support the sealing flange from buckling in use.

In one form, the seal-forming structure may comprise a compression sealing portion or a gasket sealing portion. In use the compression sealing portion, or the gasket sealing portion is constructed and arranged to be in compression, e.g. as a result of elastic tension in the positioning and stabilising structure.

In one form, the seal-forming structure comprises a tension portion. In use, the tension portion is held in tension, e.g. by adjacent regions of the sealing flange.

In one form, the seal-forming structure comprises a region having a tacky or adhesive surface.

In certain forms of the present technology, a seal-forming structure may comprise one or more of a pressure-assisted sealing flange, a compression sealing portion, a gasket sealing portion, a tension portion, and a portion having a tacky or adhesive surface.

### 5.3.1.2 Nose Bridge or Nose Ridge Region

In one form, the non-invasive patient interface 3000 comprises a seal-forming structure that forms a seal in use on a nose bridge region or on a nose-ridge region of the patient's face.

In one form, the seal-forming structure includes a saddle-shaped region constructed to form a seal in use on a nose bridge region or on a nose-ridge region of the patient's face.

### 5.3.1.3 Upper Lip Region

In one form, the non-invasive patient interface 3000 comprises a seal-forming structure that forms a seal in use on an upper lip region (that is, the lip superior) of the patient's face.

In one form, the seal-forming structure includes a saddle-shaped region constructed to form a seal in use on an upper lip region of the patient's face.

### 5.3.1.4 Chin-Region

In one form the non-invasive patient interface 3000 comprises a seal-forming structure that forms a seal in use on a chin-region of the patient's face.

In one form, the seal-forming structure includes a saddle-shaped region constructed to form a seal in use on a chin-region of the patient's face.

### 5.3.1.5 Forehead Region

In one form, the seal-forming structure that forms a seal in use on a forehead region of the patient's face. In such a form, the plenum chamber may cover the eyes in use.

### 5.3.1.6 Nasal Pillows

In one form the seal-forming structure of the non-invasive patient interface 3000 comprises a pair of nasal puffs, or nasal pillows, each nasal puff or nasal pillow being constructed and arranged to form a seal with a respective naris of the nose of a patient.

Nasal pillows in accordance with an aspect of the present technology include: a frusto-cone, at least a portion of which forms a seal on an underside of the patient's nose, a stalk, a flexible region on the underside of the frusto-cone and connecting the frusto-cone to the stalk. In addition, the structure to which the nasal pillow of the present technology is connected includes a flexible region adjacent the base of the stalk. The flexible regions can act in concert to facilitate a universal joint structure that is accommodating of relative movement both displacement and angular of the frusto-cone and the structure to which the nasal pillow is connected. For example, the frusto-cone may be axially displaced towards the structure to which the stalk is connected.

In one example of nasal pillows, at least a portion of the frusto-cone of each nasal pillow may be shaped and dimensioned to enter the corresponding naris of the patient. In another example of nasal pillows, the frusto-cone of each nasal pillow may be shaped and dimensioned so as not to enter the corresponding naris of the patient. Each nasal pillow may be configured to seal against portions of the patient's nose defining a respective naris, including the patient's columella and a respective nasal ala.

In some examples of nasal pillows, each nasal pillow may be stalkless. The frusto-cone of each nasal pillow may be attached directly to a portion of the patient-interface 3000 defining a plenum chamber 3200.

### 5.3.1.7 Nasal Mask

In one form, the non-invasive patient interface 3000 comprises a seal-forming structure 3100 that forms a seal in use to an upper lip region (e.g. the lip superior), to the patient's nose bridge or at least a portion of the nose ridge above the pronasale, and to the patient's face on each lateral side of the patient's nose, for example proximate the patient's nasolabial sulci. The patient interface 3000 shown in FIG. 1B has this type of seal-forming structure 3100. This patient interface 3000 may deliver a supply of air or breathable gas to both nares of patient 1000 through a single orifice. This type of seal-forming structure 3100 may be referred to as a “nasal cushion” and a patient interface 3000 having such a seal-forming structure 3100 may be identified as a “nasal mask”.

### 5.3.1.8 Full Face Mask

In one form the patient interface 3000 comprises a seal-forming structure 3100 that forms a seal in use on a patient's chin-region (which may include the patient's lip inferior and/or a region directly inferior to the lip inferior), to the patient's nose bridge or at least a portion of the nose ridge superior to the pronasale, and to cheek regions of the patient's face. The patient interface 3000 shown in FIG. 1C is of this type. This patient interface 3000 may deliver a supply of air or breathable gas to both nares and mouth of patient 1000 through a single orifice. This type of seal-forming structure 3100 may be referred to as a “full face cushion” and the patient interface 3000 may be identified as a “full-face mask”.

### 5.3.1.9 Ultracompact Full-Face Mask

In one form the patient interface 3000 comprises a seal-forming structure 3100 that forms a seal in use on a patient's chin region (which may include the patient's lip inferior and/or a region directly inferior to the lip inferior), to an inferior and or anterior surface of the patient's pronasale and to the patient's face on each lateral side of the patient's nose, for example proximate the nasolabial sulci. The seal-forming structure 3100 may also form a seal against a patient's lip superior. A patient interface 3000 having this type of seal-forming structure may have a single opening configured to deliver a flow of air or breathable gas to both nares and mouth of a patient, may have an oral hole configured to provide air or breathable gas to the mouth and a nasal hole configured to provide air or breathable gas to the nares, or may have an oral hole for delivering air to the patient's mouth and two nasal holes for delivering air to respective nares. This type of patient interface 3000 may be known as an ultra-compact full face mask and may comprise an ultra-compact full face cushion.

### 5.3.1.10 Nasal Cradle Mask

In one form, for example as shown in FIG. 3Z, the seal-forming structure 3100 is configured to form a seal in use with inferior surfaces of the nose around the nares. The seal-forming structure 3100 may be configured to seal around the patient's nares at an inferior periphery of the patient's nose including to an inferior and/or anterior surface of the patient's pronasale and to the patient's nasal alae. The seal-forming structure 3100 may seal to the patient's lip superior. This type of seal-forming structure 3100 may be referred to as a “cradle cushion”, “nasal cradle cushion” or “under-the-nose cushion”, for example.

The shape of the seal-forming structure 3100 may be configured to match or closely follow the underside of the patient's nose and may not contact a nasal bridge region of the patient's nose or any portion of the patient's nose superior to the pronasale. In one form of nasal cradle cushion, the seal-forming structure 3100 comprises a bridge portion dividing the opening into two orifices, each of which, in use, supplies air or breathable gas to a respective one of the patient's nares. The bridge portion may be configured to contact or seal against the patient's columella in use. Alternatively, the seal-forming structure 3100 may comprise a single opening to provide a flow or air or breathable gas to both of the patient's nares.

### 5.3.2 Plenum Chamber

The plenum chamber 3200 may be formed by a portion of the patient interface 3000 that has a perimeter that is shaped to be complementary to the surface contour of the face of an average person in the region where a seal will form in use. In use, a marginal edge of the portion of the patient interface 3000 forming the plenum chamber 3200 is positioned in close proximity to an adjacent surface of the face. Actual contact with the face is provided by the seal-forming structure 3100. The seal-forming structure 3100 may extend in use about the entire perimeter of a portion of the patient interface 3000 forming the plenum chamber 3200. In some forms, the plenum chamber 3200 and the seal-forming structure 3100 are formed from a single homogeneous piece of material.

In certain forms of the present technology, the plenum chamber 3200 does not cover the eyes of the patient in use. In other words, the eyes are outside the pressurised volume defined by the plenum chamber. Such forms tend to be less obtrusive and/or more comfortable for the wearer, which can improve compliance with therapy.

In certain forms of the present technology, the plenum chamber 3200 is formed by one or more components constructed from a transparent material, e.g. a transparent polycarbonate. The use of a transparent material can reduce the obtrusiveness of the patient interface, and help improve compliance with therapy. The use of a transparent material can aid a clinician to observe how the patient interface is located and functioning.

In certain forms of the present technology, the plenum chamber 3200 is formed by one or more components constructed from a translucent material. The use of a translucent material can reduce the obtrusiveness of the patient interface, and help improve compliance with therapy.

### 5.3.3 Positioning and Stabilising Structure

The seal-forming structure 3100 of the patient interface 3000 of the present technology may be held in sealing position in use by the positioning and stabilising structure 3300. The positioning and stabilising structure 3300 may comprise and function as “headgear” since it engages the patient's head in order to hold the patient interface 3000 in a sealing position.

In one form the positioning and stabilising structure 3300 provides a retention force at least sufficient to overcome the effect of the positive pressure in the plenum chamber 3200 to lift off the face.

In one form the positioning and stabilising structure 3300 provides a retention force to overcome the effect of the gravitational force on the patient interface 3000.

In one form the positioning and stabilising structure 3300 provides a retention force as a safety margin to overcome the potential effect of disrupting forces on the patient interface 3000, such as from tube drag, or accidental interference with the patient interface.

In one form of the present technology, a positioning and stabilising structure 3300 is provided that is configured in a manner consistent with being worn by a patient while sleeping. In one example the positioning and stabilising structure 3300 has a low profile, or cross-sectional thickness, to reduce the perceived or actual bulk of the apparatus. In one example, the positioning and stabilising structure 3300 comprises at least one strap having a rectangular cross-section. In one example the positioning and stabilising structure 3300 comprises at least one flat strap.

In one form of the present technology, a positioning and stabilising structure 3300 is provided that is configured so as not to be too large and bulky to prevent the patient from lying in a supine sleeping position with a back region of the patient's head on a pillow.

In one form of the present technology, a positioning and stabilising structure 3300 is provided that is configured so as not to be too large and bulky to prevent the patient from lying in a side sleeping position with a side region of the patient's head on a pillow.

In one form of the present technology, a positioning and stabilising structure 3300 is provided with a decoupling portion located between an anterior portion of the positioning and stabilising structure 3300, and a posterior portion of the positioning and stabilising structure 3300. The decoupling portion does not resist compression and may be, e.g. a flexible or floppy strap. The decoupling portion is constructed and arranged so that when the patient lies with their head on a pillow, the presence of the decoupling portion prevents a force on the posterior portion from being transmitted along the positioning and stabilising structure 3300 and disrupting the seal.

In one form of the present technology, a positioning and stabilising structure 3300 comprises a strap constructed from a laminate of a fabric patient-contacting layer, a foam inner layer and a fabric outer layer. In one form, the foam is porous to allow moisture, (e.g., sweat), to pass through the strap. In one form, the fabric outer layer comprises loop material to engage with a hook material portion.

In certain forms of the present technology, a positioning and stabilising structure 3300 comprises a strap that is extensible, e.g. resiliently extensible. For example the strap may be configured in use to be in tension, and to direct a force to draw a seal-forming structure into sealing contact with a portion of a patient's face. In an example the strap may be configured as a tie.

In one form of the present technology, the positioning and stabilising structure comprises a first tie, the first tie being constructed and arranged so that in use at least a portion of an inferior edge thereof passes superior to an otobasion superior of the patient's head and overlays a portion of a parietal bone without overlaying the occipital bone.

In one form of the present technology suitable for a nasal-only mask or for a full-face mask, the positioning and stabilising structure includes a second tie, the second tie being constructed and arranged so that in use at least a portion of a superior edge thereof passes inferior to an otobasion inferior of the patient's head and overlays or lies inferior to the occipital bone of the patient's head.

In one form of the present technology suitable for a nasal-only mask or for a full-face mask, the positioning and stabilising structure includes a third tie that is constructed and arranged to interconnect the first tie and the second tie to reduce a tendency of the first tie and the second tie to move apart from one another.

In certain forms of the present technology, a positioning and stabilising structure 3300 comprises a strap that is bendable and e.g. non-rigid. An advantage of this aspect is that the strap is more comfortable for a patient to lie upon while the patient is sleeping.

In certain forms of the present technology, a positioning and stabilising structure 3300 comprises a strap constructed to be breathable to allow moisture vapour to be transmitted through the strap, In certain forms of the present technology, a system is provided comprising more than one positioning and stabilizing structure 3300, each being configured to provide a retaining force to correspond to a different size and/or shape range. For example the system may comprise one form of positioning and stabilizing structure 3300 suitable for a large sized head, but not a small sized head, and another. suitable for a small sized head, but not a large sized head.

### 5.3.3.1 Conduit Headgear

### 5.3.3.1.1 Conduit Headgear Tubes

In some forms of the present technology, the positioning and stabilising structure 3300 comprises one or more headgear tubes 3350 that deliver pressurised air received from a conduit forming part of the air circuit 4170 from the RPT device to the patient's airways, for example through the plenum chamber 3200 and seal-forming structure 3100. In the form of the present technology illustrated in FIG. 3Z, the positioning and stabilising structure 3300 comprises two tubes 3350 that deliver air to the plenum chamber 3200 from the air circuit 4170. The tubes 3350 are configured to position and stabilise the seal-forming structure 3100 of the patient interface 3000 at the appropriate part of the patient's face (for example, the nose and/or mouth). This allows the conduit of air circuit 4170 providing the flow of pressurised air to connect to a connection port 3600 of the patient interface in a position other than in front of the patient's face, for example on top of the patient's head.

Since air can be contained and passed through headgear tubing in order to deliver pressurised air from the air circuit 4170 to the patient's airways, the positioning and stabilising structure 3300 may be described as being inflatable. It will be understood that an inflatable positioning and stabilising structure 3300 does not require all components of the positioning and stabilising structure 3300 to be inflatable. For example, in the example shown in FIG. 3Z, the positioning and stabilising structure 3300 comprises the tubes 3350, which are inflatable, and the strap portion 3310, which is not inflatable.

In the form of the present technology illustrated in FIG. 3Z, the positioning and stabilising structure 3300 comprises two tubes 3350, each tube 3350 being positioned in use on a different side of the patient's head and extending across the respective cheek region, above the respective ear (superior to the otobasion superior on the patient's head) to the elbow 3612 on top of the head of the patient 1000. This form of technology may be advantageous because, if a patient sleeps with their head on its side and one of the tubes is compressed to block or partially block the flow of gas along the tube, the other tube remains open to supply pressurised gas to the patient. In other examples of the technology, the patient interface 3000 may comprise a different number of tubes, for example one tube, or three or more tubes. In one example in which the patient interface has one tube 3350, the single tube 3350 is positioned on one side of the patient's head in use (e.g. across one cheek region) and a strap forms part of the positioning and stabilising structure 3300 and is positioned on the other side of the patient's head in use (e.g. across the other region) to assist in securing the patient interface 3000 on the patient's head.

In the form of the technology shown in FIG. 3Z the two tubes 3350 are fluidly connected at superior ends to each other and to the connection port 3600. In some examples, the two tubes 3350 are integrally formed while in other examples the tubes 3350 are formed separately but are connected in use and may be disconnected, for example for cleaning or storage. Where separate tubes are used they may be indirectly connected together, for example each may be connected to a T-shaped connector having two arms/branches each fluidly connectable to a respective one of the tubes 3350 and a third arm or opening providing the connection port 3600 for fluid connection to the air circuit 4170 in use.

The tubes 3350 may be formed from a flexible material, such as an elastomer, e.g. silicone or TPE, or from one or more textile and/or foam materials.

The tubes 3350 may have a preformed shape and may be able to be bent or moved into another shape upon application of a force but may return to the original preformed shape in the absence of said force. The tubes 3350 may be generally arcuate or curved in a shape approximating the contours of a patient's head between the top of the head and the nasal or oral region.

As described in U.S. Pat. No. 6,044,844, the contents of which are incorporated herein, the tubes 3350 may be crush resistant to avoid the flow of breathable gas through the tubes being blocked if either is crushed during use, for example if it is squashed between a patient's head and pillow. Crush resistant tubes may not be necessary in all cases as the pressurised gas in the tubes may act as a splint to prevent or at least restrict crushing of the tubes 3350 during use. A crush resistant tube may be advantageous where only a single tube 3350 is present as if the single tube becomes blocked during use the flow of gas would be restricted and therapy will stop or reduce in efficacy. In some examples, the tubes 3350 may be sized such that each tube 3350 is able to provide sufficient flow of gas to the plenum chamber 3200 on its own should one of the tubes 3350 become blocked.

Each tube 3350 may be configured to receive a flow of air from the connection port 3600 on top of the patient's head and to deliver the flow of air to the seal-forming structure 3100 at the entrance of the patient's airways. In the example shown in FIG. 3Z, each tube 3350 lies in use on a path extending from the plenum chamber 3200 across the patient's cheek region and superior to the patient's ear to the elbow 3612. For example, a portion of each tube 3350 proximate the plenum chamber 3200 may overlie a maxilla region of the patient's head in use. Another portion of each tube 3350 may overlie a region of the patient's head superior to an otobasion superior of the patient's head. Each of the tubes 3350 may also lie over the patient's sphenoid bone and/or temporal bone and either or both of the patient's frontal bone and parietal bone. The elbow 3612 may be located in use over the patient's parietal bone, over the frontal bone and/or over the junction therebetween (e.g. the coronal suture).

In certain forms of the present technology the patient interface 3000 is configured such that the connection port 3600 can be positioned in a range of positions across the top of the patient's head so that the patient interface 3000 can be positioned as appropriate for the comfort or fit of an individual patient. In some examples, the headgear tubes 3350 are configured to allow movement of an upper portion of the patient interface 3000 (e.g. a connection port 3600) with respect to a lower portion of the patient interface 3000 (e.g. a plenum chamber 3200). That is, the connection port 3600 may be at least partially decoupled from the plenum chamber 3200. In this way, the seal-forming structure 3100 may form an effective seal with the patient's face irrespective of the position of the connection port 3600 (at least within a predetermined range of positions) on the patient's head.

As described above, in some examples of the present technology the patient interface 3000 comprises a seal-forming structure 3100 in the form of a cradle cushion which lies generally under the nose and seals to an inferior periphery of the nose (e.g. an under-the-nose cushion). The positioning and stabilising structure 3300, including the tubes 3350 may be structured and arranged to pull the seal-forming structure 3100 into the patient's face under the nose with a sealing force vector in a posterior and superior direction (e.g. a posterosuperior direction). A sealing force vector with a posterosuperior direction may facilitate the seal-forming structure 3100 forming a good seal to both the inferior periphery of the patient's nose and the anterior-facing surfaces of the patient's face on either side of the patient's nose and the patient's lip superior.

### 5.3.3.1.2 Extendable and Non-extendable Tube Portions

In some examples of the present technology, one or both of the tubes 3350 are not extendable in length. However, in some forms, the tubes 3350 may comprise one or more extendable tube sections, for example formed by an extendable concertina structure. In some forms, the patient interface 3000 may comprise a positioning and stabilising structure 3300 including at least one gas delivery tube comprising a tube wall having an extendable concertina structure. The patient interface 3000 shown in FIG. 3Z comprises tubes 3350, the superior portions of which comprise extendable tube sections each in the form of an extendable concertina structure 3362.

The cross-sectional shape of the non-extendable tube sections 3363 of the tubes 3350 may be circular, elliptical, oval, D-shaped or a rounded rectangle, for example as described in U.S. Pat. No. 6,044,844. A cross-sectional shape that presents a flattened surface of tube on the side that faces and contacts the patient's face or other part of the head may be more comfortable to wear than, for example a tube with a circular cross-section.

In some examples of the present technology, the non-extendable tube sections 3363 connect to the plenum chamber 3200 from a low angle. The headgear tubes 3350 may extend and inferiorly down the sides of the patient's head and then curve anteriorly and medially to connect to the plenum chamber 3200 in front of the patient's face. The tubes 3350, before connecting to the plenum chamber 3200, may extend to a location at the same vertical position as or, in some examples, inferior to the connection with the plenum chamber 3200. That is, the tubes 3350 may project in an at least partially superior direction before connecting with the plenum chamber 3200. A portion of the tubes 3350 may be located inferior to the cushion module 3150 and/or the seal-forming structure 3100. The low position of the tubes 3350 in front of the patient's face facilitates contact with the patient's face below the patient's cheekbones, which may be more comfortable than contact on the patient's cheekbones and may avoid excessively obscuring the patient's peripheral vision.

### 5.3.3.1.3 Conduit Headgear Connection Port

In certain forms of the present technology, the patient interface 3000 may comprise a connection port 3600 located proximal to a superior, lateral or posterior portion of a patient's head. For example, in the form of the present technology illustrated in FIG. 3Z, the connection port 3600 is located on top of the patient's head (e.g. at a superior location with respect to the patient's head). In this example the patient interface 3000 comprises an elbow 3612 forming the connection port 3600.

The elbow 3612 may be configured to fluidly connect with a conduit of an air circuit 4170. The elbow 3612 may be configured to swivel with respect to the positioning and stabilising structure 3300 to at least partially decouple the conduit from the positioning and stabilising structure 3300. In some examples the elbow 3612 may be configured to swivel by rotation about a substantially vertical axis and, in some particular examples, by rotation about two or more axes. In some examples the elbow may comprise or be connected to the tubes 3350 by a ball-and-socket joint. The connection port 3600 may be located in the sagittal plane of the patient's head in use.

Patient interfaces having a connection port that is not positioned anterior to the patient's face may be advantageous as some patients may find a conduit that connects to a patient interface anterior to their face to be unsightly and/or obtrusive. For example, a conduit connecting to a patient interface anterior to the patient's face may be prone to interference with bedclothes or bed linen, particularly if the conduit extends inferiorly from the patient interface in use. Forms of the present technology comprising a patient interface having a connection port positioned superiorly to the patient's head in use may make it easier or more comfortable for a patient to lie or sleep in one or more of the following positions: a side-sleeping position, a supine position (e.g. on their back, facing generally upwards) or in a prone position (e.g. on their front, facing generally downwards). Moreover, connecting a conduit to an anterior portion of a patient interface may exacerbate a problem known as tube drag in which the conduit exerts an undesired force upon the patient interface during movement of the patient's head or the conduit, thereby causing dislodgement away from the face. Tube drag may be less of a problem when force is received at a superior location of the patient's head than anterior to the patient's face proximate to the seal-forming structure (where tube drag forces may be more likely to disrupt the seal).

### 5.3.3.1.4 Headgear Tube Fluid Connections

The two tubes 3350 are fluidly connected at their inferior ends to the plenum chamber 3200. In certain forms of the technology, the connection between the tubes 3350 and the plenum chamber 3200 is achieved by connection of two rigid connectors. The tubes 3350 and plenum chamber 3200 may be configured to enable the patient to easily connect the two components together in a reliable manner. The tubes 3350 and plenum chamber 3200 may be configured to provide tactile and/or audible feedback in the form of a ‘re-assuring click’ or like sound which may be easy for a patient to use as the patient may know for sure that each tube 3350 has been correctly connected to the plenum chamber 3200. In one form, the tubes 3350 are formed from a silicone or textile material and the inferior end of each of the silicone tubes 3350 is overmolded to a rigid connector made, for example, from polypropylene, polycarbonate, nylon or the like. The rigid connector on each tube 3350 may comprise a female mating feature configured to connect with a male mating feature on the plenum chamber 3200. Alternatively, the rigid connector on each tube 3350 may comprise a male mating feature configured to connect to a female mating feature on the plenum chamber 3200. In other examples the tubes 3350 may each comprise a male or female connector formed from a flexible material, such as silicone or TPE, for example the same material from which the tubes 3350 are formed.

In other examples a compression seal is used to connect each tube 3350 to the plenum chamber 3200. For example, a resiliently flexible (e.g. silicone) tube 3350 without a rigid connector may be configured to be squeezed to reduce its diameter so that it can be compressed into a port in the plenum chamber 3200 and the inherent resilience of the silicone pushes the tube 3350 outwards to seal the tube 3350 in the port in an air-tight manner. Alternatively, in a hard-to-hard type engagement between the tube 3350 and the plenum chamber 3200, each tube 3350 and/or plenum chamber 3200 may comprise a pressure activated seal, for example a peripheral sealing flange. When pressurised gas is supplied through the tubes 3350 the sealing flange may be urged against the join between the tubes and a circumferential surface around a port or connector of the plenum chamber 3200 to form or enhance a seal between the tube 3350 and plenum chamber 3200.

### 5.3.3.1.5 Conduit Headgear Straps

In certain forms of the present technology, the positioning and stabilising structure 3300 comprises at least one headgear strap acting in addition to the tubes 3350 to position and stabilise the seal-forming structure 3100 at the entrance to the patient's airways. As shown in FIG. 3Z, the patient interface 3000 comprises a strap portion 3310 forming part of the positioning and stabilising structure 3300. The strap portion 3310 may be known as a back strap or a rear headgear strap, for example. In other examples of the present technology, one or more further straps may be provided. For example, patient interfaces 3000 according to examples of the present technology having a full face cushion may have a second, lower, strap configured to lie against the patient's head proximate the patient's neck and/or against posterior surfaces of the patient's neck.

In the example shown in FIG. 3Z, strap portion 3310 of the positioning and stabilising structure 3300 is connected between the two tubes 3350 positioned on each side of the patient's head and passing around the back of the patient's head, for example overlying or lying inferior to the occipital bone of the patient's head in use. The strap portion 3310 connects to each tube above the patient's ears. With reference to FIG. 3Z, the positioning and stabilising structure 3300 comprises a pair of tabs 3355. In use a strap portion 3310 may be connected between the tabs 3355, The strap portion 3310 may be sufficiently flexible to pass around the back of the patient's head and lie comfortably against the patient's head, even when under tension in use.

### 5.3.4 Vent

In one form, the patient interface 3000 includes a vent 3400 constructed and arranged to allow for the washout of exhaled gases, e.g. carbon dioxide.

In certain forms the vent 3400 is configured to allow a continuous vent flow from an interior of the plenum chamber 3200 to ambient whilst the pressure within the plenum chamber is positive with respect to ambient. The vent 3400 is configured such that the vent flow rate has a magnitude sufficient to reduce rebreathing of exhaled CO2 by the patient while maintaining the therapeutic pressure in the plenum chamber in use.

One form of vent 3400 in accordance with the present technology comprises a plurality of holes, for example, about 20 to about 80 holes, or about 40 to about 60 holes, or about 45 to about 55 holes.

The vent 3400 may be located in the plenum chamber 3200. Alternatively, the vent 3400 is located in a decoupling structure, e.g., a swivel.

In some forms, the patient interface 3000 may comprise a vent 3400 to allow a continuous flow of gases exhaled by the patient from an interior of the plenum chamber 3200 to ambient, for example throughout the patient's entire respiratory cycle, said vent 3400 being sized and shaped to maintain the therapeutic pressure in the plenum chamber in use.

### 5.3.5 Decoupling Structure(s)

In one form the patient interface 3000 includes at least one decoupling structure, for example, a swivel or a ball and socket.

### 5.3.6 Connection Port

Connection port 3600 allows for connection to the air circuit 4170.

### 5.3.7 Forehead Support

In one form, the patient interface 3000 includes a forehead support. In some examples the forehead support may be a portion of, or connected to, a frame 3700.

### 5.3.8 Anti-Asphyxia Valve

In one form, the patient interface 3000 includes an anti-asphyxia valve.

In some examples the AAV is provided at a connection port 3600 of the patient interface 3000 or a connection between the plenum chamber 3200 and a short tube 3610, such as an inlet port connector.

### 5.3.9 Ports

In one form of the present technology, a patient interface 3000 includes one or more ports that allow access to the volume within the plenum chamber 3200. In one form this allows a clinician to supply supplementary oxygen. In one form, this allows for the direct measurement of a property of gases within the plenum chamber 3200, such as the pressure.

5.3.10 Cushion Module and Portions thereof

In some examples of the present technology, the patient interface 3000 comprises a cushion module 3150. The cushion module 3150 may be formed by the components forming the plenum chamber 3200, seal-forming structure 3100 and, in some examples, additional components or portions.

FIG. 7 shows a cushion module 3150 in contact with a patient's face. In this example the cushion module 3150 comprises a chassis portion 3210 partially forming the plenum chamber 3200. In this example the seal-forming structure 3100 also partially forms the plenum chamber 3200. The chassis portion 3210 and seal-forming structure 3100 together form the plenum chamber 3200 by enclosing a volume of space (e.g., breathing cavity) which can be filled with air at a therapeutic pressure, such as of at least 4 cmH₂O or at least 6 cmH₂O above ambient air pressure.

The cushion module 3150 may form a patient interface 3000 with other components such as a frame and a positioning and stabilising structure 3300 as shown in FIG. 3A. The cushion module 3150 in FIG. 7 is separable from other components of the patient interface 3000. More generally, in some examples of the present technology the patient interface 3000 comprises a removable cushion module 3150.

In some examples, the cushion module 3150 may be replaced in the patient interface 3000 by another cushion module 3150, for example one of a different size (or at least having a seal-forming structure 3100 having a different size or shape).

In other examples the cushion module 3150 may not be separable from other components or portions of the patient interface 3000, such as the positioning and stabilising structure 3300. In some examples the cushion module 3150 may comprise a chassis portion 3210 at least part of which is integrally formed with either or both of the headgear tubes 3350 or integrally formed with portions of the positioning and stabilising structure. Unless the context clearly requires otherwise, features of the patient interfaces 3000 disclosed herein are to be understood as being applicable whether the patient interface has a chassis portion 3210 that is part of a removable cushion module 3150 or not.

The plenum chamber 3200 may include one or more plenum chamber inlet ports 3240 sized and structure to receive a flow of air at the therapeutic pressure for breathing by the patient.

In the example shown in FIG. 7, the plenum chamber 3200 comprises one plenum chamber inlet port 3240. In particular, the chassis portion 3210 defines an opening, configured to be connected to a fluid connector (e.g. an elbow as shown in FIG. 3A, or the like) to which an air circuit 4170 can be connected. This opening forms the plenum chamber inlet port 3240.

As will be described in detail, cushion modules 3150 of patient interfaces 3000 according to examples of the present technology may also comprise cushions 3130. It is to be understood that while in some examples of the present technology a cushion 3130 may form only a small part of a component, that component may still be identified as a cushion module 3150.

FIGS. 8A-8B, 10, 12, 13A-13B, and 14A-14C show other cushion modules 3150 of patient interfaces 3000 according to the present technology. The cushion modules 3150 shown in FIGS. 7-10 and 12 are nasal-only cushion modules 3150, leaving the patient's mouth uncovered. These cushion modules may comprise a single hole and may comprise a seal-forming structure 3100 configured to seal to the patient's nasal ridge and sides of the nose, the patient's cheeks and the patient's upper lip. The cushion modules 3150 shown in FIGS. 11, 13A-13B and 14A-14C are configured to seal around both the patient's nose and mouth. A cushion module 3150 that seals to the patient's nose and mouth may comprise an oral hole 3271 through which the pressurised flow of air may be provided to the patient's mouth, and one or more nasal holes 3272 through which the pressurised flow of air may be provided to the patient's nasal airways. The cushion module 3150 may comprise a single nasal hole 3272 as shown in FIGS. 13A-13B or may comprise two nasal holes 3272 configured to provide a flow of air at positive pressure to respective nares, as shown in FIGS. 14A-14C. The seal-forming structure 3100 may comprise a nasal portion 3110 and an oral portion 3120. The nasal portion 3110 may be configured to seal around the patient's nasal airways in use. The oral portion 3120 may be configured to seal around the patient's mouth in use.

### 5.3.11 Connections to Positioning and Stabilising Structure

The patient interface 3000 may comprise a positioning and stabilising structure 3300 to provide a force to hold the seal-forming structure 3100 in a therapeutically effective position on the patient's head. The positioning and stabilising structure 3300 may comprise one or more straps configured to attach to respective sides of the plenum chamber 3200 and pass around the back of the patient's head.

As described above, the chassis portion 3210 and seal-forming structure 3100 may in some examples form a cushion module 3150. FIGS. 7-10 and 12 all show cushion modules 3150. These cushion modules 3150 may be configured to connect to the positioning and stabilising structure 3300 as shown in FIG. 3A. In the example shown in FIG. 3A, the patient interface comprises a frame 3700. The frame 3700 is configured to connect the positioning and stabilising structure 3300 to the cushion module 3150. In these examples the cushion module 3150 is removably attached to the frame 3700, although in other examples the frame 3700 may be permanently attached the cushion module 3150.

It is to be understood that in some examples, the patient interface 3000 does not comprise a forehead support. For example, the forehead support of the frame 3700 shown in FIG. 3A may be replaced with a pair of upper arms 3311 as shown in FIGS. 13A-13B, each upper arm 3311 extending at least superiorly and laterally such that upper straps 3313 of the positioning and stabilising structure 3300, similar to the upper straps 3313 shown in FIG. 11, may connect to the upper arms 3311.

In some examples the patient interface 3000 does not comprise a frame 3700 as a separable component and instead the cushion module 3150 may comprise a pair of upper arms 3311, as shown in FIGS. 13A-13B. The upper arms 3311 may connect to upper straps 3313 similar to those shown in FIG. 11. The upper straps 3313 may lie superior to an otobasion superior of the patient's head in use and may each pass between a respective eye and ear of the patient's head.

As shown in FIG. 11, the positioning and stabilising structure 3300 may further comprise a pair of lower straps 3317 configured to connect to the cushion module 3150 or a frame on either lateral side of the patient interface 3000. The lower straps 3317 may lie inferior to an otobasion inferior of the patient's head in use.

In other examples, the patient interface may comprise a positioning and stabilising structure 3300 comprising a pair of gas delivery tubes 3350 configured to provide the flow of air at therapeutic pressure the plenum chamber 3200 and configured to provide a force to hold the seal-forming structure 3100 in sealing position. FIG. 3Z shows an example of such a positioning and stabilising structure 3300.

It is to be understood that the cushion modules 3150 and components and portions thereof may be applied in patient interfaces 3000 having any type of positioning and stabilising structure 3300.

5.3.12 Seal-Forming Structure having a Cushion

Some forms of the present technology comprise a patient interface 3000 comprising a seal-forming structure 3000 comprising a cushion 3130. The cushion 3130 may be deformable and resilient, e.g. it may provide a cushioning function when engaged with the patient's face.

The cushion 3130 may be at least partially formed by a lattice structure.

The patient interfaces 3000 or cushion modules 3150 shown in FIGS. 7-14C have cushions 3130 formed from lattice structures.

In some forms of the technology the cushion 3130 is airtight and forms a seal directly to the surface of the patient's face. FIG. 9 shows such an example.

In other forms of the technology, the cushion 3130 is at least partially covered by another portion of the seal-forming structure 3100 which seals to the surface of the patient's face, while the cushion 3130 provides a cushioning function. FIGS. 7, 8A-8C and 10-14C show examples of such a patient interface 3000. In these examples, the seal-forming structure may comprise a face engaging membrane 3118. The face engaging membrane 3118 may be configured to contact the face and may be flexible and resilient.

The face engaging membrane 3118 may at least partially cover the cushion 3130 in use. In some examples, such as those shown in FIGS. 12 and 14A-14C, the face engaging membrane 3118 may cover the cushion 3130 and be in contact with or lie very close to the cushion 3130 when the seal-forming structure 3100 is not engaged with a patient's face. As shown in FIGS. 8A-8C, for example, in some forms at least a portion of the face engaging membrane 3118 may be separated from the cushion 3130 when seal-forming structure 3100 is not engaged with a patient's face, but may contact and at least partially cover the cushion 3130 in use when the seal-forming structure engages the patient's face, as shown in FIGS. 7 and 10 for example.

In some examples, such as those shown in FIGS. 7-14C, the patient interface comprises a chassis portion 3210 at least partially forming the plenum chamber 3200. The seal-forming structure 3100 may be attached to and supported by the chassis portion 3210, which may be stiffer than the seal-forming structure 3100. As shown in cross section in FIGS. 7, 8A-8C, 9, 10 and 12 for example, the chassis portion 3210 is significantly thicker than the face engaging membrane 3118. In the example of FIG. 9, the cushion 3130 is more flexible than the chassis portion 3210 such that in use the chassis portion 3210 supports the cushion 3130 as the cushion 3130 deforms while engaging the patient's face. In these examples the seal-forming structure 3100 (e.g. face engaging membrane 3118 and/or cushion 3130 as the case may be), are attached to a perimeter of a chassis portion 3210 and the chassis portion 3210 defines the majority of the volume of the plenum chamber 3200.

As shown in FIGS. 7, 8A-8C, 9, 10, and 12, by way of example, the face engaging membrane 3118 may extend from the chassis portion 3210. The face engaging membrane 3118 may be formed from an elastomeric material, such as silicone or TPE for example. In some examples the chassis portion 3210 is also formed from an elastomeric material, such as silicone or TPE, and may be formed from the same elastomeric material as the face engaging membrane 3118. In some examples the chassis portion 3210 and face engaging membrane 3118 may be integrally formed, for example moulded together by injection moulding in a single moulding step/shot. In other examples the face engaging membrane 3118 may be overmoulded to the face engaging membrane 3118. In further examples the chassis portion 3210 may be formed from a material that is substantially rigid during normal use, such as polycarbonate or similar, and may have thinner walls in comparison to an elastomeric chassis portion 3210 such that it may be identified as a shell. In further examples the face engaging membrane 3118 may be formed from a textile material, for example a textile material backed with an air-impermeable layer, which may be formed from silicone or TPU, for example.

In some examples the chassis portion 3210 may be formed from an elastomeric material and have a thickness of 2 mm or more, such as 3 mm, 3.5 mm or 4 mm or more. In some examples the face engaging membrane 3118 may be formed from an elastomeric material and may have a thickness of 1.5 mm or less, such as 1 mm, 0.75 mm, 0.5 mm or 0.3 mm or less. In some examples the face engaging membrane 3118 may have a thickness 0.25 mm or 0.2 mm.

In the examples shown in FIGS. 7, 8A-8C, 9, 10 and 13A-13B and 14A-14C, the cushion 3130 is positioned interior to the plenum chamber 3200. The plenum chamber 3200 in the vicinity of the cushion 3130 is instead defined by the chassis portion 3210 and/or the face engaging membrane 3118. Advantageously this means that the cushion 3130 does not need to be made airtight and can be formed from a lattice structure with open cells, which may provide different stiffness characteristics to a sealed lattice structure.

In another example, shown in FIG. 9, the cushion 3130 is not positioned entirely interior to the plenum chamber 3200 but instead partially defines the plenum chamber 3200. In this example there is no face engaging membrane 3118. The cushion 3130 is connected to the chassis portion 3210 around a perimeter of the plenum chamber 3200. The cushion 3130 provides both a cushioning function and also forms a seal to the patient's face. The cushion 3130 in this example is airtight, at least at the surfaces defining the plenum chamber 3200 and forming a seal to the patient's face. The cushion 3130 may be formed from a lattice structure but may have an airtight outer layer. The airtight outer layer may be applied to the lattice structure, e.g. as a coating, for example formed from an elastomeric material such as silicone or TPE, or may be integrally formed with the lattice structure. Advantageously, an integrally formed airtight layer may be formed during the same process by which the lattice structure of the cushion 3130 is formed, such as 3D printing or injection moulding.

In another form, such as the examples shown in FIGS. 11 and 12, the cushion 3130 is positioned exterior to the plenum chamber 3200. That is, the cushion 3130 is outside of the path of the flow of air through the plenum chamber 3200. Advantageously, this arrangement may make the surfaces defining the plenum chamber 3200 easier to clean as the plenum chamber 3200 can be defined by uniform surfaces, such as of the chassis portion 3210 and face engaging membrane 3118. These uniform surfaces are also unobstructed by the cushion 3130. Furthermore, the cushion 3130 may be less susceptible to becoming unclean as it is not in contact with the patient's exhalate and associated moisture and any other fluids. In the examples shown in FIGS. 11 and 12, a cushion 3130 is connected to the chassis portion 3210 and face engaging membrane 3118 but is attached to external surfaces thereof. As shown in FIG. 12 for example, the face engaging membrane 3118 covers a patient-facing side, which in this example is a side of the cushion 3130 facing towards the plenum chamber 3200. In this example the face engaging membrane 3118 wraps around the cushion 3130 to partially cover the cushion 3130 from the inside (e.g. from the plenum chamber 3200 side). In these examples the cushion 3130 may be positioned such that in use it is able to deform against the chassis portion 3210. Advantageously the chassis portion 3210 is stiff and holds the cushion 3130 in position and provides a less deformable structure for the cushion 3130 to deform against.

In some examples the cushion 3130 comprises one or more clips by which it attaches to the chassis portion. In other examples the cushion 3130 is glued or welded to the chassis portion and/or the face engaging membrane 3118, or attached in another suitable manner.

### 5.3.12.1 Cushion Forming Heat and Moisture Exchanger

As described above, in some examples at least a portion of the cushion 3130 is provided within the plenum chamber 3200. Additionally, in some examples, at least some of the flow of air passing through the plenum chamber 3200 passes through the lattice structure forming the cushion 3130. FIG. 8C shows a cushion module 3150 comprising a cushion 3130 in which the flow of air delivered to the patient passes through the cushion 3130. Like other cushions 3130 disclosed herein, the cushion 3130 may be formed from a lattice structure. The lattice structure may be uncovered or comprise uncovered portions and, being permeable to air, may therefore allow for air to flow from one side to the other.

In some examples, the cushion 3130 may primarily function only as a cushion 3130 but, by the nature of the shape and/or size of the plenum chamber 3200 and the cushion 3130, the cushion 3130 may be positioned within the plenum chamber 3200 such that air flows through it within the plenum chamber 3200. In other examples, such as the example shown in FIG. 8C, the cushion 3130 also forms a heat and moisture exchanger (HMX). The HMX may function to increase the temperature and/or humidity of the flow of air before being delivered to the patient. The cushion 3130 may absorb some heat and moisture from gas exhaled by the patient into the plenum chamber 3200. The cushion 3130 may then impart heat and moisture to the flow of air delivered to the plenum chamber 3200 prior to inhalation by the patient. In some examples, the cushion 3130 may function as an HMX, may be formed by a lattice structure and may also function as a cushion 3130 for the seal-forming structure 3100.

In the example shown in FIG. 8C, the cushion 3130 at least partially covers the plenum chamber inlet port 3240. This requires or at least encourages incoming air flow to pass through the cushion 3130. The cushion module 3150 shown in FIG. 8C may be intended for use in a patient interface 3000 such as the one shown in FIG. 3A, but the vent 3400 of the patient interface 3000 may be provided upstream of the plenum chamber inlet port 3240, such as in the connector defining connection port 3600. Therefore, the patient's exhalate must pass back through the plenum chamber inlet port 3240 before reaching the vent 3400, imparting heat and moisture to the lattice structure forming the cushion 3130 on its way. In other examples the vent 3400 may be formed by or provided to one or more walls forming the plenum chamber 3200, downstream of the plenum chamber inlet port 3240 but upstream of the cushion 3130. That is, the vent 3400 may be provided between the plenum chamber inlet port 3240 and the cushion 3130. In such an example some or all of the exhalate may pass through the cushion 3130 but be discharged through the vent 3400 before it is able to pass back through the plenum chamber inlet port 3240.

In the example shown in FIG. 8C, the cushion 3130 fills a majority of the plenum chamber 3200. The cushion 3130 may be structured to fill at least a majority of the dead space that would otherwise exist once portions of the patient's nose are received within the interior of the cushion module 3150 in use. It is to be understood that references to the cushion 3130 filling space within the plenum chamber 3200 are references to the overall size of the cushion, being the overall volume bounded by the outer peripheral surfaces of the cushion 3150, e.g. the volume of material actually occupied by the lattice structure plus the volume of the voids defined by the lattice structure.

In examples in which the cushion 3130 fills at least a majority of the plenum chamber 3200 and/or at least partially covers the plenum chamber inlet port 3240, including examples in which the cushion 3130 forms an HMX, the cushion 3130 may be provided with one or more holes, peripheral channels or other open passages configured to allow free flow of air to and from the patient's airways in the event the lattice structure forming the cushion 3130 becomes unintentionally occluded, for example clogged by excess fluid, moisture etc. as a result of unintended operating conditions, misuse or any other reason. Despite such openings or channels the cushion 3130, during normal operating conditions, may still function as an HMX to at least improve the temperature and/or humidity of the air delivered to the patient's airways.

While the cushion module 3150 shown in FIG. 8C is a nasal cushion, a cushion 3130 formed from a lattice structure and forming an HMX may be provided within any suitable type of cushion module 3150, such as for a full face mask (e.g. the type of patient interface 3000 shown in FIG. 11, an ultra compact full-face mask (e.g. the type of patient interface 3000 shown in FIGS. 13A-13B and 14A-14C), a nasal cradle mask (e.g. the type shown in FIG. 3Z) or any other suitable patient interface 3000 in which a cushion 3130 may be provided, including a patient interface 3000 comprising nasal pillows.

A cushion 3130 that also forms an HMX may comprise a lattice structure formed from a material and/or formed with a particular structure that encourages heat and/or moisture to be absorbed from exhalate and released to the flow of air delivered to the patient's airways. In some examples the lattice structure is formed from a material able to absorb moisture. For example, the lattice structure may be formed from a fibrous material, paper, suitable foams or the like. In one form the lattice structure may be formed by injection moulded or 3D printed paper pulp. In other examples the physical structure of the lattice structure, e.g. the shape and size of unit cells, or general shape and size of the portions forming a stochastic/random lattice, may encourage condensation of moisture onto, and subsequent evaporation from, the surface of the lattice structure material. In some examples the lattice structure may function as an HMX by both absorbing moisture into the material forming the HMX and also by being conducive to condensation of moisture onto its surface. In examples in which it is the structure of the lattice structure that encourages condensation and evaporation to provide the HMX function, the lattice structure may be formed from any suitable material disclosed here, by any suitable process. In further examples, the lattice structure forming the cushion 3130 may impregnated with moisture absorbent material in order to form an HMX.

The cushion 3130 may be removable from the patient interface 3000, for example for cleaning or replacement. This may be particularly useful for a cushion 3130 that also forms an HMX, since regular cleaning or replacement may be required more often than the cushion module 3150 or patient interface 3000 is required to be replaced. It is also to be understood that cushions 3130 described herein that do not also form an HMX may be removable.

While in the FIG. 8C example the cushion 3130 is formed from a lattice structure and also forms an HMX, in some examples a lattice structure disclosed herein may be used to form an HMX that is not a cushion 3130 and instead functions only or primarily as an HMX. For example, such a component may not engage the user's face in use.

### 5.3.13 Lattice Structure

As stated above, the cushion 3130 may be formed by a lattice structure.

As labelled in FIGS. 8B, 8C, 9, 15-18 and 23A-23B for example, the cushion 3130 may comprise a cushion body 3131 formed by a lattice structure. That is, the material forming the cushion 3130 is structured and arranged to form a lattice. The lattice structure may comprise a plurality of unit cells. Where features or properties of a cushion 3130 are described, it is to be understood that those features or properties may be features or properties of a cushion body 3131 and not features or properties of other portions of a cushion 3130, such as any cushion clips or added layers, unless context requires otherwise. For example, a cushion 3130 may be described as being formed by a lattice structure if the cushion body 3131 has a lattice structure, despite the cushion 3130 having cushion clips or an outer layer that are not formed by a lattice structure.

In some examples, as shown in FIG. 7-1, the lattice structure may be formed by a plurality of interconnected struts 3166 that form a plurality of voids 3168. The structure of the struts 3166 may repeat in two or three dimensions to form a plurality of unit cells that make up the lattice. In examples, each void may be considered as the empty space defined by each unit cell. Such lattice structure may be advantageous in providing a relatively lightweight, flexible and breathable structure. In examples, the cushion 3130 may comprise 20 or more voids.

In use, as shown in FIG. 7-2, the lattice structure may provide flexibility to conform to facial features and/or accommodate anthropometric variation. When the patient interface is in engagement with the patient's face, the struts 3166 may flex thereby altering the size, shape and/or orientation of the voids to allow the cushion 3130 to conform to the patient's face. Additionally, as described later in this disclosure, characteristics of the lattice structure may vary in different portions of the lattice to adjust the stiffness or flexibility of the cushion for different areas of the patient's face. For example, stiffness and flexibility may be adjusted by changing or varying the material of the struts, thickness of the struts, density of the struts, orientation of the struts, spacing of the struts, size of the voids, orientation of the voids, density of the voids, arrangement of unit cells, and/or density of unit cells.

The lattice structure is distinguishable from foam materials where the cell/pore structure is formed on a microscopic level typically with an inflating agent. The lattice structure has a repeating macroscopic cellular structure built up by the material forming the struts.

In some examples of the present technology, the lattice structure may be 3D printed. In some particular examples, the lattice structure may be 3D printed in a shape corresponding to a unique patient's face, for example using any one of the personalisation or automatic sizing techniques described herein. In other examples, the lattice structure may be formed by another additive manufacturing technique or another manufacturing technique able to produce a lattice structure, or the lattice structure may be formed by injection moulding. In some examples the lattice structure may be formed by knitting in a form similar to a spacer fabric. In some examples the lattice structure may be woven. In an alternative example, instead of a lattice structure, the cushion may resemble bubble wrap.

In some examples, the lattice structure is formed from an elastomeric material. The lattice structure is formed from silicone or from TPE. In some examples, the lattice structure is formed from TPU. In examples, the lattice structure is formed from a material having a Durometer hardness within the range of 20 Shore A to 80 Shore A. In other examples, depending on geometry, the hardness may be within the range of 15-100 Shore A. Other ranges envisaged are 15-50, 30-80, 30-60, 20-50, and 20-40 Shore A, for example. In one form the hardness is 30 Shore A.

Various possible lattice structures are envisioned. In some examples, the lattice structure comprises a two-dimensional structure (e.g. honeycomb). In further examples, the lattice structure comprises a three-dimensional structure. In examples, the lattice structure may comprise a fluorite structure (shown in FIG. 23A), a truncated cube structure (shown in FIG. 23B), a IsoTruss structure (shown in FIG. 23C), a hexagonal honeycomb structure (shown in FIG. 23D), a gyroid structure (shown in FIG. 23E) or a Schwarz structure (shown in FIG. 23F). In some examples the cushion 3130 may be formed from another lattice structure. In some examples the cushion 3130 may be formed from a plurality of lattice structures.

In some examples of the present technology, the cushion 3130 is formed flat and bent into a three-dimensional shape during assembly with the face engaging membrane 3118. For example, in some forms the cushion 3130 may be 3D printed in a flat configuration. The cushion 3130 may be flexible and able to assume a three-dimensional shape corresponding to a curvature along the length of the face engaging membrane 3118. In other examples, the cushion 3130 may be formed in a three-dimensional shape. In some examples, the cushion 3130 may be 3D printed in a three-dimensional curved shape. A cushion 3D printed in a three-dimensional curved shape may be customised, as will be described.

In some other examples, the lattice structure may be knitted. In further examples, the lattice structure may be formed from foam having holes formed therein to form a lattice structure. The holes may be formed by laser cutting, for example.

FIG. 18 shows schematically a foam cushion 3130 having a cushion body 3131 having a plurality of holes 3136 formed in the cushion body 3131. Forming the holes 3136 removes material from the cushion body 3131 to effectively form it into a lattice structure (although in some examples the cushion body 3131 may be formed, e.g. moulded, with the holes already present meaning no material removal may be required) or at least into a shape such that the cushion body 3131 behaves like a lattice structure. In some examples, the size, shape and/or spacing of the holes 3136 varies along a length of the cushion 3130 and/or between a first side of the cushion 3130 and a second side of the cushion 3130. In some examples the size, shape, and/or spacing of the holes 3136 may be selected to provide a cushion 3130 having one or more properties tailored to a specific user, for example based on facial data representing one or more features of the user's face.

A cushion 3130 formed by a lattice structure may advantageously be able to be designed and produced with fine control over certain properties, at least in comparison to a uniform mass of foam in some examples. In examples of the present technology, the lattice structure forming the cushion 3130 can be designed and produced with greater softness or compliance in specific regions of contact on the face where compliance is more desirable than other regions. The properties of a cushion 3130 formed from a uniform mass of foam for example may be more dependent on the overall cross sectional shape and overall stiffness, making it more difficult to achieve fine control of properties in particular locations, whereas properties of a lattice structure may be varied at specific locations within the cushion 3130, in examples of the present technology. In some examples, the lattice structure may be configured to optimise contact pressure based on tissue spring rate and expected dynamic movements. In some examples, the cushion 3130 may apply pressure for a static seal whilst the face engaging membrane 3118 provides an inflatable dynamic seal.

Some patients may dislike the idea of a foam cushion in their patient interface 3000, due to perceived issues with cleanability. In examples of the present technology, a cushion 3130 formed from a lattice structure may be at least as comfortable as a foam cushion but may be more appealing to those patients that dislike foam. In some examples the lattice structure is open cell and formed from a machine washable material. In some examples the entire cushion module 3150 may be formed from machine washable or sterilisable materials (e.g. face engaging membrane 3118 formed from silicone, lattice structure formed from TPU or silicone, chassis portion 3210 formed from silicone or polycarbonate or the like) meaning the cushion module 3150 can be disconnected from the rest of the patient interface 3000 and washed or sterilised easily.

### 5.3.13.1 Varying Properties

In some forms of the present technology, the cushion 3130 may comprise one or more characteristics that vary between different locations at which the seal-forming structure 3100 engages the patient's face. As an example, the one or more varying characteristics may include stiffness of the cushion 3130. That is, the cushion 3130 may be stiffer in a portion corresponding to one location on the patient's face than in a portion corresponding to another location on the patient's face. The one or more varying characteristics may include characteristics of the lattice structure, such as any one or more of shape, thickness, density, spacing, relative orientation and/or material of unit cells forming the lattice structure.

It is to be understood that the way that a lattice structure may be configured to provide for a stiffer region in one location in comparison to another location will depend on the particular lattice structure. In some examples the lattice structure may comprise a beam lattice structure, e.g. a lattice structure formed by a network of members behaving as beams or struts. In one such example the cushion 3130 may comprise a first region and a second region, the first region being stiffer due to increased thickness and/or density of struts. In another example a cushion 3130 may comprise a lattice structure formed from bendable beams. In a first region there may be a large number of readily bendable beams while in a second region there may be a small number of relatively non-bendable beams, such that the first region is more compliant or less stiff than the second region. It is to be understood that multiple parameters may be available for modification throughout a lattice structure to provide different behaviour in different regions of the cushion 3130. In some examples, more voids may be provided in a region of a lattice structure having a lesser stiffness than in a region having a higher stiffness.

In general, where a cushion 3130 is described herein as being more compliant or less stiff in one region in comparison to another, or stiffer in one region in comparison to another, the particular parameters/characteristics of the lattice structure from which that cushion 3130 is constructed may be varied as required to provide the desired differences in behaviour between the regions.

In some examples, the lattice structure may be provided with characteristics resulting in more complex behaviour than only being stiff or flexible. For example, in some forms of the technology the lattice structure may be formed from struts, but some or all of the struts may be curved (C-shaped or otherwise). The curved struts may bend in a controlled manner. In other examples some or all of the struts may be straight and may be configured to buckle in use. For example, the struts may function to resist load directed along their length, until a point at which the struts may buckle. At the time of buckling the stiffness of struts may be reduced and the struts may allow for further compression without excessive stiffness. Such an arrangement may provide for a long “travel” (e.g. high adaptability) without unduly large force required between the user's face and the cushion 3130.

In some examples, the lattice structure may be constructed to define voids which close (or move towards a more closed position) during compression of the cushion 3130. Upon closure of substantially all voids in a region, the cushion 3130 may stiffen significantly as it may have no more capacity for compression. In some examples the number, size, shape of the voids available may be selected to cause such stiffening in certain regions where a large amount of support may be required.

### 5.3.13.1.1 Variation in Nose-Only Masks

In some examples, the patient interface 3000 may comprise a seal-forming structure 3100 configured to seal to the patient's face at the patient's lip superior, on lateral sides of the patient's nose and at the patient's nasal ridge. This type of patient interface 3000 may be known as a nasal mask into which the tip of the patient's nose is inserted. FIGS. 7-10 and 12 show examples of cushion modules 3150 suitable for a nasal masks. In these examples the cushion 3130 is positioned around an entire periphery of the patient's nose in use, although in other examples the cushion 3130 may be positioned only within particular portions of the seal-forming structure 3100.

The cushion 3130 may comprise a lip superior portion provided within a portion of the seal-forming structure 3100 configured to seal to the patient's face at the patient's lip superior. In some examples, the cushion 3130 may comprise side of nose portions provided within respective portions of the seal-forming structure 3100 configured to seal to the patient's face at the sides of the patient's nose. In some examples, the cushion 3130 is stiffer at the sides of nose portions than at the lip superior portion. In some further examples the cushion 3130 may comprise a nasal ridge portion provided within a portion of the seal-forming structure 3100 configured to seal to the patient's face at the patient's nasal ridge. The cushion 3130 may be stiffer in some examples at the side of nose portions than at the nasal ridge portion. The cushion 3130 may advantageously be less stiff at the nasal ridge than at other portions because the nasal ridge may be a sensitive area and also a surface with high curvature where a high level of compliance is desirable to avoid leaks.

In another form, the seal-forming structure 3100 is configured to seal to the patient's face at the patient's lip superior, between the nasal alae and the nasolabial sulci, and at an inferior periphery of the patient's nose including the nasal alae and a pronasale region of the patient's nose. This type of patient interface 3000 may be known as a nasal cradle mask, under-the-nose mask or the like. The seal-forming structure 3100 may be configured not to engage the patient's nasal ridge. In such a patient interface 3000 the seal-forming structure 3100 may comprise either one or two nasal holes 3272.

The seal-forming structure 3100 may comprise a lip superior portion provided within a portion of the seal-forming structure 3100 configured to seal to the patient's face at the patient's lip superior. The cushion 3130 may further comprise a pair of posterior corner portions provided within a portion of the seal-forming structure 3100 configured to seal to the patient's face between the nasal alae and the nasolabial sulci. The cushion 3130 may be stiffer in the posterior corner portions than in the lip superior portion. In some examples the cushion 3130 may comprise an inferior nose periphery portion provided within a portion of the seal-forming structure configured to seal to the patient's face at the inferior periphery of the patient's nose.

In some particular examples, the cushion 3130 the cushion is stiffer in the posterior corner portions than in the inferior nose periphery portion.

### 5.3.13.1.2 Variation in Nose-and-Mouth Masks

In some examples, the patient interface 3000 may comprise a seal-forming structure 3100 that is configured to seal to the patient's face at the patient's lip inferior, at the patient's cheeks, on the lateral sides of the patient's nose and at the patient's nasal ridge. This type of patient interface 3000 may be known as a full face mask that seals around both the nose and mouth, and into which the tip of the patient's nose is inserted. FIG. 11 shows such a patient interface 3000. The seal-forming structure 3100 in this example comprises a single hole configured to provide the flow of air to both the nose and mouth of the patient. In this example the cushion 3130 is positioned around an entire periphery of the patient's nose and mouth in use, although in other examples the cushion 3130 may be positioned only within particular portions of the seal-forming structure 3100.

The cushion 3130 may comprise a lip inferior portion provided within a portion of the seal-forming structure 3100 configured to seal to the patient's face at the patient's lip inferior. In some forms, the cushion 3130 may comprise a pair of cheek portions provided within respective portions of the seal-forming structure 3100 configured to seal to the patient's face at the patient's cheeks. In some examples, the cushion 3130 may be stiffer in the cheek portions than in the lip inferior portion. The cushion 3130 may comprise side of nose portions provided within respective portions of the seal-forming structure 3100 configured to seal to the patient's face on the lateral sides of the patient's nose. In some examples the cushion 3130 comprises a nasal ridge region provided within a portion of the seal-forming structure 3100 configured to seal to the patient's face at the patient's nasal ridge. The cushion 3130 may be stiffer in the side of nose portions than the nasal ridge portion, in some examples. The cushion 3130 may advantageously be less stiff at the nasal ridge than at other portions because the nasal ridge may be a sensitive area and also a surface with high curvature where a high level of compliance is desirable to avoid leaks.

In some examples, such as those shown in FIGS. 13A-13B and 14A-14C, the seal-forming structure 3100 is configured to seal to the patient's face at the patient's lip inferior, at the patient's cheeks, at the patient's lip superior, and at an inferior periphery of the patient's nose including the nasal alae and a pronasale region of the patient's nose. This type of patient interface 3000 may be known as a compact full-face mask, ultra-compact full face mask, minimal-contact full face mask or an under-the-nose full face mask or the like. The patient interface 3000 may be configured to not engage the nasal ridge. The seal-forming structure 3100 may comprise a pair of nasal holes 3272 each configured to provide the flow of air to a respective one of the patient's nares, as shown in FIG. 14A-14C, or a single nasal hole 3272 configured to provide the flow of air to both nares as shown in FIGS. 13A-13B. The seal-forming structure 3100 may further comprise an oral hole to provide the flow of air to the patient's mouth, as shown in FIGS. 13A-13B and 14A-14C. In further examples the seal-forming structure 3100 may comprise a single hole to provide the flow of air to both nose and mouth of the patient. The cushion 3130 may be positioned around the entire periphery of the patient's nose and mouth or may only be positioned in particular portions of the seal-forming structure 3100.

The cushion 3130 may comprise a lip inferior portion provided within a portion of the seal-forming structure 3100 configured to seal to the patient's face at the patient's lip inferior, as shown in FIGS. 13A-13B and 14A-14C for example. The cushion 3130 may comprise cheek portions provided within respective portions of the seal-forming structure 3100 configured to seal to the patient's face at the patient's cheeks, as shown in FIGS. 13A-13B and 14A-14C for example. In some particular examples, the cushion 3130 is stiffer in the cheek portions than in the lip inferior portion. As shown in FIGS. 14A-14C, the cushion 3130 is provided only within a lip inferior portion and cheek portions of the seal-forming structure 3100.

The cushion 3130 may further comprise a lip superior portion provided within a portion of the seal-forming structure 3100 configured to seal to the patient's face at the patient's lip superior. In some examples, the cushion 3130 comprises an inferior nose periphery portion provided within a portion of the seal-forming structure 3100 configured to seal to the patient's face at the inferior periphery of the patient's nose. In some examples the cushion 3130 may further comprise a pair of posterior corner portions provided within a portion of the seal-forming structure 3100 configured to seal to the patient's face between the nasal alae and the nasolabial sulci. The cushion 3130 may be stiffer in the lip superior portion and/or the posterior corner portions than in the inferior nose periphery portion. Advantageously, the cushion 3130 may have lower stiffnesses at the inferior nose periphery and/or lip inferior than other regions, as these may be sensitive and/or may have complex surface geometry requiring a high level of compliance. As shown in FIG. 13A the cushion 3130 is provided within a lip inferior portion and cheek portions of the seal-forming structure 3100 and also extends into the lip superior portion of the seal-forming structure 3100 although does intersect the sagittal plane in use.

5.3.13.1.3 Variation between Patient-Facing and Non-Patient Facing Sides

In addition, or as an alternative, to variation between locations on the patient's face, in some examples the lattice structure comprises one or more characteristics that vary between a patient-facing side (also identified as a user-facing side) of the cushion 3130 corresponding to a side of the seal-forming structure 3100 configured to contact the patient's face in use and a non-patient facing side (also identified as a non-user facing side) of the cushion 3130 corresponding to a side of the seal-forming structure 3100 configured to face away from the patient's face in use. FIGS. 15A-15B and 16-18 schematically show cushions 3130 with this manner of variation in the lattice structure.

In some forms, the lattice structure on the patient-facing side of the cushion is configured to be highly compliant, comfortable and/or structured and arranged to avoid leaving red marks on the patient's face. The lattice structure on the non-patient facing side of the cushion may be configured to adapt readily to the overall size and shape of the patient's face. As shown by way of example in FIG. 15A, the lattice structure may comprise smaller voids 3168 on the patient-facing side than on the non-patient facing side. Small voids may present a low risk of face marking on the patient-facing side while the large voids on the non-patient facing side may allow the lattice structure of the seal-forming structure 3100 to readily adapt to different face shapes and sizes (e.g., by having increased flexibility due to the relatively larger voids). Facial marking may be considered unsightly and/or embarrassing and may also be associated with discomfort during use. In some examples, the lattice structure may comprise smaller unit cells on the patient-facing side than on the non-patient facing side.

FIG. 15B shows another example schematically in which a cushion 3130 comprises a cushion body 3131 formed by a lattice structure defining voids 3168. In this example the voids 3168 are smaller on a patient-facing side than on a non-patient facing side. The lattice structure comprises progressively smaller voids in the direction from the non-patient facing side to the patient facing side. The density of the cushion 3130 in this example progressively increases towards the patient-facing side, eventually forming a uniform surface 3133 on the patient-facing side to provide for a smooth and comfortable interface.

The variation in the one or more characteristics of the lattice structure may make the cushion 3130 more compliant or less stiff on the patient-facing side of the cushion than on the non-patient facing side of the cushion. Such an arrangement may advantageously allow the patient-facing side to readily conform to complex geometry on the surface of the patient's face (e.g. to form a good seal) while on the non-patient facing side the cushion is able to resist compressive forces and make larger adjustments to adapt to the overall shape and size of the patient's face.

As shown by way of example in FIGS. 16 and 17, the material (e.g., the struts 3166) forming the unit cells of the lattice structure may be thinner on the patient-facing side of a cushion 3130 than on the non-patient facing side of the cushion 3130. In some examples, the material forming the unit cells of the lattice structure has a thickness within the range of 0.3-0.5 mm on the patient-facing side of the cushion 3130. In a particular example, the material forming the unit cells of the lattice structure has a thickness within the range of 0.8 mm-1.2 mm, such as a thickness of approximately 1 mm on the non-patient facing side of the cushion 3130.

In some examples, such as in the cushion 3130 shown in FIG. 16, the patient-facing side of the cushion 3130 (e.g. the cushion body 3131 thereof) is defined by unit cells of the lattice structure. The unit cells may be exposed to contact the face engaging membrane 3118. The cushion 3130 in this example, or at least the patient-facing side thereof, may advantageously be highly compliant. In this example the cushion 3130 may comprise a uniform surface 3132 defining the non-patient-facing side of the cushion 3130. In other examples the cushion 3130 may not comprise a uniform surface 3132 on the non-patient facing side and instead the unit cells may be exposed.

In other examples, such as in the cushion 3130 shown in FIG. 17, the cushion 3130 comprises a uniform surface 3133 on the patient-facing side of the cushion 3130. The uniform surface may cover unit cells of the lattice structure. This may advantageously provide for a low risk of facial marking during use.

In some examples, the uniform surfaces 3132 and/or 3133 on the patient-facing side and/or non-patient facing side, as the case may be, may be integrally formed with unit cells of the lattice structure. For example, the uniform surfaces 3132 and/or 3133 may be 3D printed together with and connected to the lattice structure.

In other examples, the uniform surfaces 3132 and/or 3133, as the case may be, may be formed separately and attached to the lattice structure. In some examples the uniform surface 3133 on the non-patient facing side may be provided only in one or more predetermined locations, for example where the cushion 3130 abuts another portion of the cushion module 3150, e.g. a chassis portion 3210.

As described above, in some examples the lattice structure may be formed from foam having a plurality of holes formed therein (e.g. holes in the macroscopic structure of the cushion 3130 distinct from the microscopic cells/pores of the foam material). The holes may be formed by laser cutting, for example or the cushion 3130 may be formed, e.g. moulded, with holes. The size, shape and/or spacing of the holes may be varied within the cushion 3130 in order to vary one or more properties (e.g. stiffness, compliance). FIG. 18 shows schematically a foam cushion 3130 having a cushion body 3131 having a plurality of holes 3136 formed in the cushion body 3131. The holes 3136 in this example are smaller on a first side (left side in FIG. 18) than on a second side (right side in FIG. 18) of the cushion 3130, to provide the first side of the cushion 3130 with a different stiffness than the second side 3130. Alternatively or additionally, the holes 3136 may vary in size, shape and/or spacing along the length of the cushion 3130. In the example shown in FIG. 18 the holes are circular but in other examples the holes may have a shape other than a circle.

### 5.3.13.1.4 Variation Proximate Sensitive Facial Feature

In some examples of the present technology, the cushion 3130 may comprise a lattice structure which comprises one or more characteristics that vary at and/or proximate a location corresponding to a sensitive facial feature on the user's face. A variation in characteristics may, for example, be a variation in any one or more of the shape, thickness, density, spacing, relative orientation and/or material of unit cells forming the lattice structure.

FIG. 19A shows schematically a cushion 3130 in contact with a patient's face in the region of a sensitive facial feature and on either side of the sensitive facial feature. The sensitive facial feature may be a protruding/raised facial feature, such as a nose bridge, pronasale or cheek bone, for example. The cushion 3130 is receiving a uniformly distributed load on the non-patient facing side of the cushion 3130, which for example may be a load transmitted to the cushion 3130 from tension in headgear straps of the positioning and stabilising structure 3300 transmitted through components of the patient interface 3000. FIG. 19B shows a plot of the force or contact pressure on the patient's face in the region shown in FIG. 19A.

Two curves are plotted in FIG. 19B. The solid line curve represents force or pressure applied to the user's face by a cushion 3130 with a uniform lattice structure (which may be identified as an “unoptimised” cushion 3130). As shown in FIG. 19B, there is an increase in the force/pressure applied to the face by the unoptimised cushion 3130 at and proximate the sensitive facial feature, due to the extra compression of the lattice structure caused by the protruding/raised facial feature. The broken line represents force or pressure applied to the user's face by a cushion 3130 having one or more characteristics that vary at or proximate the sensitive facial feature. This cushion 3130 may be considered an “optimised” cushion 3130. The term “optimised” is to be understood to mean “more optimal” in the context of some outcome, such as comfort, stability etc. As shown in FIG. 19B, there is no increase in force/pressure on the user's face at the sensitive facial feature, due to the varying characteristics of the lattice structure. In one example, the lattice structure may be configured to have a lesser stiffness in the region configured to contact the sensitive facial feature than the in the regions on either side of the sensitive facial feature. The lesser stiffness in this illustrated example then results in the force between the sensitive facial feature and the cushion 3130 being no more than the force between the other regions and the cushion 3130.

More generally, the variation in the one or more characteristics of the lattice structure may cause the cushion 3130 to apply less pressure on the sensitive facial feature in use than would be applied without the variation. In FIG. 19B, this is shown by the optimised cushion 3130 applying a lesser force on the face at the sensitive facial feature than the unoptimised cushion 3130. The optimised cushion 3130 applies a more uniform load over the surface of the user's face, as some of the load that would otherwise be applied to the sensitive facial feature is instead applied to the user's face on either side of the sensitive facial feature (where the face is less sensitive). As a result, less load is applied to the sensitive facial feature, in comparison to the unoptimised cushion 3130, despite the cushion 3130 as a whole bearing the same load. This may advantageously adequately support the patient interface 3000 on the user's face while maintaining a good seal and without creating a sore point at the sensitive facial feature.

In further examples, the variation of the one or more characteristics may cause the cushion 3130 to apply less pressure on the sensitive facial feature in use than the cushion 3130 applies to the user's face around the sensitive facial feature. The variation of the one or more characteristics of the lattice structure may result in greater compliance in the cushion 3130 at and/or proximate the location corresponding to the sensitive facial feature. FIG. 20A shows schematically a cushion 3130 in contact with a user's face in the region of a sensitive facial feature and on either side of the sensitive facial feature. The cushion 3130 is receiving a uniformly distributed load on the non-user facing side of the cushion 3130. FIG. 20B shows a plot of the force or contact pressure on the user's face in the region shown in FIG. 20A.

The solid line curve in FIG. 20B is substantially the same as the solid line curve in FIG. 19B and represents the force/pressure across the user's face applied by an unoptimised cushion 3130, showing an increase in force at the sensitive facial feature. The broken line curve in FIG. 20B represents the force/pressure across the user's face applied by an optimised cushion 3130 according to another example of the present technology. In this example, despite the presence of a raised sensitive facial feature, the variation in the lattice structure causes the force applied to the user's face at the sensitive facial feature to be less than that applied on either side of the sensitive facial feature. Advantageously, this may provide for a particularly comfortable seal-forming structure 3100 as almost all the load is applied to the less sensitive regions around the sensitive facial feature. While in this example and in the example of an optimised cushion 3130 described with reference to FIG. 19B, the optimised cushion 3130 applies a greater load on either side of the sensitive facial feature than the non-optimised cushions 3130, i.e. the optimisation of the lattice structure increases the force on either side of the sensitive facial feature, this may be desirable as these regions may be able to more comfortably support loads than the region of the sensitive facial feature. The load at the sensitive facial feature may be limited to the force required to prevent leaks at the sensitive facial feature, or close to it with a factor of safety.

FIG. 19C shows an example of a cushion 3130 having a lattice structure with a characteristic that varies along the length of the cushion 3130. In this example the cushion 3130 comprise uniform surfaces 3132 and 3133 on the non-patient facing and patient-facing sides of the cushion 3130, respectively. The cushion 3130 further comprises a recess 3134 configured to engage a sensitive facial feature such as a nose bridge, cheek bone or the like. The grid-like pattern depicting the cushion body 3131 represents the lattice structure schematically. In this example the orientation of cells forming the lattice structure vary proximate the recess 3134 to provide for a different behaviour at and proximate the recess 3134. For example, the variation in the orientation of the lattice structure at and proximate the recess 3134 may result in a lesser stiffness at the recess 3134, which may in turn provide for comfortable engagement between the cushion 3130 and the sensitive facial feature.

FIG. 22C shows another example of a cushion 3130 comprising a lattice structure with a variation in a characteristic of the lattice structure configured to provide for user comfort. As illustrated schematically, the cushion 3130 comprises a stiffened region 3139 within the cushion being stiffer than one or more adjacent regions within the cushion (e.g. the left and right side regions of the cushion 3130, as well as a region between the stiffened region 3139 and the recess 3134). In this example the stiffened region 3139 is positioned to span from a first region of the cushion 3130 located on a first side of the sensitive facial feature (e.g. the left side in FIG. 22C) through a second region of the cushion 3130 overlying the sensitive facial feature and into a third region of the cushion 3130 on a second side of the sensitive facial feature (e.g. the right side in FIG. 22C). The stiffened region 3139 may be stiffened by a variation in one or more characteristics of the lattice structure at the stiffened region 3139. As illustrated in FIG. 22C the lattice structure is denser in the stiffened region 3139 than a surrounding compliant region 3138. The increased density may be formed by, for example, more material, smaller voids, additional struts and/or smaller and more numerous unit cells, for example. The actual parameter(s) which may be varied to increase stiffness will depend on the particular lattice structure used in various examples.

FIG. 22D shows yet another example of a cushion 3130 comprising a lattice structure with a variation in a characteristic of the lattice structure resulting in less stiffness in the cushion 3130 at and around a location corresponding to a sensitive facial feature than other regions. In this example, the cushion 3130 comprises a stiffened region 3139 and a compliant region 3138 (i.e., the compliant region having greater flexibility/less stiffness as compared to the stiffened region), each formed by variation in characteristics of the lattice structure, such as variation in one or more of shape, thickness, density, spacing, relative orientation and/or material of unit cells forming the lattice structure. Also, in this example the stiffened region 3139 spans from a first region (e.g. on the left in FIG. 22D) of the cushion 3130 on a first side of the sensitive facial feature through a second region of the cushion 3130 overlying the sensitive facial feature and into a third region (e.g. on the right in FIG. 22D) of the cushion 3130 on a second side of the sensitive facial feature. In this example the cushion 3130 is stiffer proximate the patient's face in the first region and in the third region than in the second region. That is, the stiffened region 3139 is provided all the way up to the side of the cushion 3130 which engages the patient's face in use in the regions on either side of the sensitive facial feature. Additionally, the cushion 3130 comprises a compliant region 3138 surrounding the sensitive facial feature, configured to provide a region of lesser stiffness at the sensitive facial feature for comfort, while the stiffened region 3139 is stiffer to transfer a majority of the overall force on the cushion 3130 to the less sensitive regions of the patient's face on either side of the sensitive facial feature.

FIGS. 22G-22J show, schematically, four different ways a lattice structure formed from a network of struts around voids may be configured, in order to provide different stiffness and adaptability. Each shows a cushion 3130 comprising a cushion body 3131 comprising a lattice structure.

In FIG. 22G the lattice structure is formed from relatively thick struts 3166 spaced relatively far apart from each other (high relative spacing) thereby forming relatively large voids 3168. This structure may provide for a cushion 3130 having a medium stiffness and high adaptation distance.

In FIG. 22H the lattice structure is formed from relatively thin struts 3166 spaced relatively far apart from each other (high relative spacing) thereby forming relatively large voids 3168. This structure may provide for a cushion 3130 having a low stiffness and high adaptation distance.

In FIG. 22I the lattice structure is formed from relatively thick struts 3166 spaced relatively close to each other (low relative spacing) thereby forming relatively small voids 3168. This structure may provide for a cushion 3130 having a high stiffness and lower adaptation distance.

In FIG. 22J the lattice structure is formed from relatively thin struts 3166 spaced relatively close to each other (low relative spacing) thereby forming relatively small voids 3168. This structure may provide for a cushion 3130 having a medium stiffness and medium adaptation distance. As shown in FIG. 22J, a patient-facing side of the cushion 3130 may comprise a uniform surface 3133 that may be thicker than the struts to provide for a comfortable surface in use. In some examples the uniform surface 3133 may be between 1.5-3 times as thick as the thinnest portion of some or all of the struts, such as between 1.7 and 2.5 times as thick, such as twice as thick.

As described elsewhere herein, a cushion 3130 may comprise a lattice structure with one or more characteristics that vary to provide for differing properties in different locations within the cushion 3130. FIGS. 22C and 22D, described above in more detail, comprise stiffened regions 3139 and compliant regions 3138 formed by variations in characteristics of the lattice structures, such as strut thickness.

FIG. 22E shows a further example of a cushion 3130 comprising a cushion body 3131 comprising a lattice structure. The cushion is shown schematically against a patient's face. The patient's face has a protrusion, which represents a sensitive facial feature such as a nose bridge or other sensitive feature. In this example, the cushion 3130 comprises a stiffened region 3139 on each lateral side of the sensitive facial feature, connected to each other proximate a non-patient facing side of the cushion 3130. The stiffened regions 3139 may be formed, for example, by the structure shown in FIG. 22I, as this structure provides a high stiffness. Proximate the sensitive facial feature, the cushion 3130 comprises a compliant region 3138, which may be less stiff than the stiffened regions 3139. The compliant region 3138 may be formed from the structure shown in FIG. 22H, as this structure provides a low stiffness and high adaptation/compliance distance. Along the patient-facing side of the cushion 3130, the cushion body 3131 may be formed by the lattice structure shown in FIG. 22J, which has a medium stiffness and medium adaptation distance. The smaller voids and thinner struts may provide for a comfortable feel against the patient's face. The cushion 3130 may comprise a uniform layer of material along the patient-facing side to provide for a smooth surface.

FIG. 22F shows a further example of a cushion 3130 similar to that shown in FIG. 22E. In this example, the compliant region 3138 formed by the structure shown in FIG. 22H extends all of the way to the surface of the patient-facing side of the cushion 3130 at the sensitive facial feature. The structure (being that of FIG. 22J) that is provided along the patient-facing side of the cushion 3130 shown in FIG. 22E is, in FIG. 22F, not provided directly over the sensitive facial feature and is instead provided on either lateral side of the sensitive facial feature. The compliant region 3138 formed by the structure shown in FIG. 22H extending substantially all the way to the surface of the cushion 3130 may allow the patient-facing side of the cushion 3130 to be highly stretchable in directions parallel to the surface (indicated by the arrows in FIG. 22F) at the sensitive facial feature. The structure with smaller voids (shown in FIG. 22J) provided along the patient-facing surface of the cushion 3130 may allow less stretch in the surface.

While certain variations in a lattice structure forming a cushion 3130 are described above (and elsewhere) in the context of reducing load on a sensitive facial feature, it is to be understood that the variations may be applied in any cushion 3130 which has one or more regions stiffer than one or more other regions, even where there is not one specific facial feature considered to be a sensitive facial feature.

FIG. 21A shows schematically another example of a cushion 3130 comprising a lattice structure in contact with a user's face in the vicinity of a raised/protruding sensitive facial feature such as a nose bridge, pronasale, cheek bone or the like. In this example the load on the non-user facing side of the cushion 3130 is a non-uniform distributed load. As illustrated, the distributed load is greater on the left side than on the right side. There is also a recess 3134 in the cushion 3130, which will be described in more detail below.

FIG. 21B shows a plot of force/pressure across the user's face in the vicinity of the sensitive facial feature. The solid-line curve shows the force on the face exerted by an unoptimized cushion 3130 receiving the non-uniform distributed load shown in FIG. 21A. As shown in FIG. 21B, the force transmitted to the face is large on the left side of the sensitive facial feature and smaller on the right side thereof, corresponding to the non-uniform distributed load applied to the cushion 3130. However, the broken-line curve in FIG. 21B shows the force transmitted to the face by an optimised cushion 3130. As illustrated, the force applied to the face on either side of the sensitive facial feature is substantially the same due to variation in the lattice structure forming the cushion 3130, despite the non-uniform distributed load applied to the non-user facing side of the cushion 3130.

Evident in the examples described with reference to FIGS. 19A-22D, is that in some examples in which the lattice structure comprises one or more characteristics that vary along a length of the cushion 3130, the cushion 3130 may receive a distributed load applied to a non-user facing side of the cushion 3130, and yet due to the variation in the lattice structure, the cushion 3130 may apply a different distributed load to the user's face along the length of the cushion 3130. In some particular examples, the cushion 3130 may receive a non-uniform distributed load along said length of the cushion 3130 applied to a non-user facing side of the cushion 3130, and yet due to the variation in the one or more characteristics the cushion 3130 applies a uniform load to the user's face along said length of the cushion. Advantageously, the cushion 3130 may be optimised to receive a non-uniform distributed load but apply a smoothed, more even (e.g. closer to uniform) load to the user's face which has a maximum force less than a maximum force of the distributed load on the user's face. This may make the patient interface 3000 particularly comfortable to wear while still able to form a good seal in use.

### 5.3.13.2 Features Proximate Sensitive Facial Features

FIG. 21A schematically shows a cushion 3130 in an undeformed state as it is being brought into contact with a user's face at and on either side of a sensitive facial feature, which may be a nose bridge, pronasale, cheek bone or other raised or sensitive feature. The cushion 3130 in this example comprises a recess 3134 configured to be aligned in use with the sensitive facial feature. The recess 3134 may be shaped to receive the sensitive facial feature, as shown in FIG. 21A.

In some examples the recess 3134 is shaped to provide a clearance between the cushion 3130 and the sensitive facial feature in an undeformed state, as shown in FIG. 21A. That is, in an undeformed state the recess may be larger than the sensitive facial feature such that the cushion 3130 does not contact the sensitive facial feature. However, in use when the cushion 3130 is pulled into contact with the user's face the cushion 3130 may compress and conform to the user's face such that there is no longer clearance at the recess 3134 and a good seal to the sensitive facial feature is formed. However, the presence of the recess 3134 and its clearance in an undeformed state may result in a particularly low force being applied on the sensitive facial feature in use, since there may be minimal compression of the cushion in the region of the recess 3134, or at least less compression than if there was no recess 3134.

In other examples the recess 3134 may not be so large that there is a clearance around the sensitive facial feature even in an undeformed state. The recess 3134 may substantially match a shape of the sensitive facial feature, for example, or may even be smaller than the sensitive facial feature. However, the presence of even a small recess 3134 may go some way to reducing the force applied to sensitive facial feature, as the recess 3134 may act as a relief, reducing some amount of compression required of the cushion 3130 at the sensitive facial feature. Advantageously the recess 3134 may provide for a particularly comfortable patient interface 3000.

FIG. 21B shows two force/pressure curves across the user's face in the vicinity of the sensitive facial feature. The solid-line curve shows the force applied to the user's face by an unoptimized cushion 3130 without a recess 3134 and the broken-line curve shows the force applied to the user's face by an optimised cushion 3130 also having a recess 3134 as shown in FIG. 21A. As shown in FIG. 21B the force transmitted to the sensitive facial feature by the cushion 3130 with the recess 3134 is less than the force transmitted to the sensitive facial feature by the cushion 3130 without the recess 3134. Furthermore, at least partially due to the recess 3134 the force applied to the sensitive facial feature by the cushion 3130 with the recess 3134 is less than the force applied to the user's face on either side of the sensitive facial feature.

In some examples of the present technology, the cushion 3130 may comprise one or more force redistribution features configured to in use redirect forces received on a non-user facing side of the cushion 3130 in a region of the cushion 3130 aligned with a sensitive facial feature into one or more regions of the cushion 3130 alongside or spaced from the sensitive facial feature. A force redistribution feature may be a variation in a characteristic of a lattice structure or may be an additional or alternative feature to a lattice structure property.

FIG. 22A schematically shows a cushion 3130 according to a further example of the present technology in contact with a user's face in the vicinity of a sensitive facial feature and receiving a distributed load on a non-user facing side thereof. FIG. 22B shows a plot of the force or pressure applied to the user's face by the cushion 3130 in use. The cushion 3130 shown in FIG. 22A comprises a force redistribution feature in the form a beam structure 3137 within the cushion 3130 (e.g. internal of the cushion body 3131 of the cushion 3130). The beam structure 3137 is positioned to in use span from a first region A of the cushion 3130 located on a first side of the sensitive facial feature through a second region B of the cushion 3130 overlying the sensitive facial feature and into a third region C of the cushion 3130 on a second side of the sensitive facial feature.

The beam structure 3137 is configured to redirect forces received at region B, which is aligned with the sensitive facial feature, into regions A and C, which are alongside and spaced from the sensitive facial feature. As shown in FIG. 22B the force transmitted to the user's face in region B, at the location of the sensitive facial feature, is lower than the force transmitted to the user's face in regions A and C, due to the beam structure 3137 redirecting forces to the regions A and C where they are able to be better tolerated by the user. In this example the cushion 3130 also comprises a recess 3134 like the example shown in FIGS. 21A and 21B, which also has an effect on reducing the force applied to the sensitive facial feature. However, as shown in FIG. 22B the reduction in force throughout region B may be more substantial and may affect a wider region (e.g. substantially all of region B) than the reduction in force resulting from the presence of the recess 3134 alone.

In some examples the cushion 3130 comprises a void adjacent the beam structure 3137 on a user-facing side of the beam structure 3137 in region B but not in regions A and C, which may result the beam structure 3137 transferring force received at region B into the cushion 3130 at regions A and C. Additionally or alternatively, the cushion 3130 may have a lesser stiffness in region B (e.g. due to variations in the lattice structure), which may also enable force to be more readily transferred to regions A and C of the cushion 3130 instead of at region B.

FIG. 22C, already discussed above in the context of a lattice structure having regions of differing stiffnesses, shows another example of a cushion 3130 with a force redistribution feature. In this example the force redistribution feature comprises the stiffened region 3139 within the cushion 3130, being stiffer than one or more adjacent regions within the cushion 3130. In this example the stiffened region 3139 is stiffened by a variation in one or more characteristics of the lattice structure at the stiffened region 3139. As illustrated schematically, the stiffened region 3139 is positioned to span from a first region of the cushion 3130 located on a first side of the sensitive facial feature (e.g. the left side in FIG. 22C) through a second region of the cushion 3130 overlying the sensitive facial feature and into a third region of the cushion 3130 on a second side of the sensitive facial feature (e.g. the right side in FIG. 22C). As illustrated in FIG. 22C the lattice structure is denser in the stiffened region 3139 than a surrounding compliant region 3138. The increased density may be formed by, for example, more material, smaller voids, additional struts and/or smaller and more numerous unit cells, for example. The actual parameter(s) which may be varied to increase stiffness will depend on the particular lattice structure used in various examples. The stiffened region 3139 in this example provides a similar effect to the beam structure 3137 described above and may function like a beam to protect the sensitive facial feature. The stiffened region 3139 may form a force redistribution feature to transmit loads on cushion 3130 at least partially away from the sensitive facial feature and into the adjacent regions which engage less sensitive areas. The stiffened region 3139 may be formed from a finer or denser lattice structure and the surrounding compliant region(s) 3138 may be formed from a coarser, less dense lattice structure to provide less stiffness and reduce weight. The surface layers of the cushion 3130 in this example may be formed from the same material as the lattice structure or may be a different material, such as textile, foam, silicone, etc.

FIG. 22D already discussed above in the context of a lattice structure having regions of differing stiffnesses, shows another example of a cushion 3130 with a force redistribution feature. In this example, the cushion 3130 comprises a stiffened region 3139 and a compliant region 3138, each formed by variation in characteristics of the lattice structure, such as variation in one or more of shape, thickness, density, spacing, relative orientation and/or material of unit cells forming the lattice structure. The stiffened region 3139 in this example forms a force redistribution feature. Also in this example, the stiffened region 3139 spans from a first region (e.g. on the left in FIG. 22D) of the cushion 3130 on a first side of the sensitive facial feature through a second region of the cushion 3130 overlying the sensitive facial feature and into a third region (e.g. on the right in FIG. 22D) of the cushion 3130 on a second side of the sensitive facial feature. In this example the cushion 3130 is stiffer proximate the patient's face in the first region and in the third region than in the second region. That is, the stiffened region 3139 is provided all the way up to the side of the cushion 3130 which engages the patient's face in use in the regions on either side of the sensitive facial feature. Additionally, the cushion 3130 comprises a compliant region 3138 surrounding the sensitive facial feature, configured to provide a region of lesser stiffness at the sensitive facial feature for comfort. In this example, the stiffened region 3139 is stiffer to form a force redistribution feature that transfers a majority of the overall force on the cushion 3130 to the less sensitive regions of the patient's face on either side of the sensitive facial feature. The portion of the stiffened region 3139 that spans between the two side regions may act as a bridge connecting the portions of the stiffened region 3139 on the sides. The central, bridge or beam-like portion may transfer load to either side of the sensitive facial feature while the side portions may form the main load paths to transfer force to the less sensitive regions on either side of the sensitive facial feature. In some examples the bridge-like central portion of the stiffened region 3139 may be stiffer than the side portions of the stiffened region 3139. It is to be understood that in some examples there are three or more regions of differing stiffnesses within the cushion 3130.

### 5.3.13.3 Personalisation

In some examples, the lattice structure of the cushion 3130 is 3D printed in a size or shape corresponding to a unique user's face. For example, facial data, which may represent a three-dimensional shape of some or all of a user's face, or one or more characteristics of a user's face, may be obtained using known methods or methods described herein (e.g. with reference to FIGS. 24, 25 and 26A-26D). The lattice structure may then be 3D printed in a shape corresponding to the user's face based on the facial data.

In some examples, the lattice structure may be formed with thicknesses (e.g. overall thickness of the cushion body 3131) based on the intended user's facial data. For example, the relative thicknesses of the cushion 3130 in various regions corresponding to different regions of the patient's face may be determined based on the facial data.

Advantageously, 3D printing of a lattice structure may be particularly suited to personalisation based on unique facial data, as it may be cost effective to produce a cushion having a customised shape, at least in comparison to other techniques such as injection moulding. Forming a cushion 3130 from foam, as described with reference to FIG. 18, for example, may also be suited to personalisation as the size, shape, spacing, positions and/or number of holes may be easily varied based on facial data during a laser cutting or other computer controlled cutting operation to produce optimal or tailored size and performance characteristics.

In some examples, the lattice structure of the cushion 3130 is constructed to optimise contact pressure for a unique individual. The lattice structure may be constructed based on facial data corresponding to the unique individual such that the cushion 3130 provides less contact pressure in one or more regions than it would without use of the facial data. The lattice structure may be tuned to optimise contact pressure in use for a particular user.

In some examples, the cushion 3130 comprises one or more personalised characteristics and is formed in a three-dimensional curved shape based on facial data. In other examples the cushion 3130 comprises one or more personalised characteristics based on facial data and is formed in a flat shape. It is to be understood that in some examples the cushion 3130 may not be personalised and may be formed in either a three-dimensional curved shape or a flat shape.

The resulting comfort and/or performance of a cushion 3130 formed (e.g. 3D printed) to a three-dimensional personalised shape may be better than cushion 3130 produced flat and without personalisation, although a cushion 3130 produced flat may be considered useful for some applications as it may be able to be provided at a lower cost.

In some examples, the cushion 3130 may be formed in a flat configuration but may have one or more features or characteristics that are personalised such as the overall thickness of the cushion 3130 or one or more properties of the lattice structure, such as thickness, spacing, density, shape, size, orientation etc. of the unit cells forming the lattice structure. A cushion 3130 formed in a three-dimensional curved shape may additionally or alternatively be personalised in the three-dimensional curved profile of the cushion 3130 (e.g. the space curve along the length of the cushion 3130).

Either or both of the overall (e.g. macroscopic) shape of the cushion 3130 and characteristics of the lattice structure forming the cushion 3130 may be personalised based on facial data. FIGS. 19A-22D show examples of “optimised” cushions 3130 having features configured to avoid excessive forces being applied to a sensitive facial feature. In some examples of the present technology, facial data of a unique user's face may include details identifying shape and/or location of a sensitive facial feature (e.g. nose bridge, cheek bone or other sensitive area). The facial data may then be used to personalise a cushion 3130 such that it behaves in the manner described with reference to any of FIGS. 19A-22D. For example, the facial data may be used to form a lattice structure with varying characteristics in the construction of the unit cells such that the lattice structure has a lower stiffness in a correct region, corresponding to the vicinity of the sensitive facial feature, than in other regions. Alternatively, or additionally, the facial data may be used to form a recess in the correct location and/or having a correct/sufficient size to correspond to the sensitive facial feature of the particular user from which the facial data has been acquired.

### 5.4 Automatic Sizing

Seal-forming structures 3100 (which may also be known as “facial interfaces”, “interfaces”, “user interfaces” and the like) according to examples of the present technology (e.g. the examples shown in FIGS. 7-22D or in any other example disclosed herein), may be provided in a range of sizes so that patients or clinicians can select a most optimal size from the range of sizes when purchasing or prescribing a patient interface 3000. Described below are systems and methods to assist users in determining the correct or most optimal size seal-forming structure 3100. It is to be understood that in some examples the systems and methods may be applied to selection of sub-components of a seal-forming structure 3100, such as a cushion 3130 (e.g. formed from a lattice structure) or other components of a patient interface 3000, such as a positioning and stabilising structure 3300. It is also to be understood that in practice the seal-forming structure 3100 may form part of a removable cushion module 3150, meaning that size selection or customisation of a seal-forming structure 3100 may require selecting or customising a cushion module 3150 having the seal-forming structure 3100. Furthermore, references to sizing of an interface are to be understood to alternatively be references to sizing of a cushion 3130 formed from a lattice structure for the seal-forming structure, or cushion module 3150 comprising such a cushion 3130.

In a beneficial embodiment, the present technology may employ an application downloadable from a manufacturer or third party server to a smartphone or tablet with an integrated camera. When launched, the application may provide visual and/or audio instructions. When prompted or otherwise, the user may activate a process using an image sensor (such as a camera function) to scan or capture one or more images of the user's face, and a facial interface size may be recommended based on an analysis of the captured image or video by a processor of the phone or a cloud. In an alternative embodiment, instead of capturing images of a subject in real-time, the user may be prompted to select and/or upload a pre-exiting image of the user's face for image processing and analysis for sizing. In one example, the image is a 2D image of the user's face. In another example, the image is a 3D image (i.e. contains depth information on selected portion) of the face. This may allow for a correct or optimal size of the facial interface identified quickly and conveniently for a user which improves user fit and comfort.

As described further below, the present technology allows a user to capture an image or series of images of their facial structure. Instructions provided by an application stored on a computer-readable medium, such as when executed by a processor, detect various facial landmarks within the images, measure and scale the distance between such landmarks, compare these distances to a data record, and recommend an appropriate facial interface size. Thus, an automated device of a consumer may permit accurate facial interface selection, such as in the home, to permit customers to determine sizing without trained associates or fitting.

### 5.4.1 System

FIG. 24 depicts an example system 200 that may be implemented for automatic facial feature measuring and facial interface sizing. System 200 may generally include one or more of servers 210, a communication network 220, and a computing device 230. Server 210 and computing device 230 may communicate via communication network 220, which may be a wired network 222, wireless network 224, or wired network with a wireless link 226. In some versions, server 210 may communicate one-way with computing device 230 by providing information to computing device 230, or vice versa. In other embodiments, server 210 and computing device 230 may share information and/or processing tasks. The system may be implemented, for example, to permit automated purchase of facial interfaces where the process may include automatic sizing processes described in more detail herein. For example, a customer may order a facial interface online after running a facial interface selection process that automatically identifies a suitable facial interface size by image analysis of the customer's facial features.

### 5.4.1.1 Computing Device

Computing device 230 can be a desktop or laptop computer 232 or a mobile device, such as a smartphone 234 or tablet 236. FIG. 25 depicts the general architecture 300 of computing device 230. Device 230 may include one or more processors 310. Device 230 may also include a display interface 320, user control/input interface 331, sensor 340 and/or a sensor interface for one or more sensor(s), inertial measurement unit (IMU) 342 and non-volatile memory/data storage 350.

Sensor 340 may be one or more cameras (e.g., a CCD charge-coupled device or active pixel sensors) that are integrated into computing device 230, such as those provided in a smartphone or in a laptop. Alternatively, where computing device 230 is a desktop computer, device 230 may include a sensor interface for coupling with an external camera, such as the webcam 233 depicted in FIG. 24. Other exemplary sensors that could be used to assist in the methods described herein that may either be integral with or external to the computing device include stereoscopic cameras, for capturing three-dimensional images, or a light detector capable of detecting reflected light from a laser or strobing/structured light source. In one embodiment, the sensor 340 comprises an Apple iphone's 3D TrueDepth Camera or similar sensors employed in other mobile devices capable of 3D facial scanning.

User control/input interface 331 allows the user to provide commands or respond to prompts or instructions provided to the user. This could be a touch panel, keyboard, mouse, microphone, and/or speaker, for example.

Display interface 320 may include a monitor, LCD panel, or the like to display prompts, output information (such as facial measurements or interface size recommendations), and other information, such as a capture display, as described in further detail below.

Memory/data storage 350 may be the computing device's internal memory, such as RAM, flash memory or ROM. In some embodiments, memory/data storage 350 may also be external memory linked to computing device 230, such as an SD card, server, USB flash drive or optical disc, for example. In other embodiments, memory/data storage 350 can be a combination of external and internal memory. Memory/data storage 350 includes stored data 354 and processor control instructions 352 that instruct processor 310 to perform certain tasks. Stored data 354 can include data received by sensor 340, such as a captured image, and other data that is provided as a component part of an application. Processor control instructions 352 can also be provided as a component part of an application.

### 5.4.1.2 Application for Facial Feature Measuring and Facial Interface Sizing

One such application is an application for facial feature measuring and/or facial interface sizing 360, which may be an application downloadable to a mobile device, such as smartphone 234 and/or tablet 236. The application 360, which may be stored on a computer-readable medium, such as memory/data storage 350, includes programmed instructions for processor 310 to perform certain tasks related to facial feature measuring and/or facial interface sizing. The application also includes data that may be processed by the algorithm of the automated methodology. Such data may include a data record, reference feature, and correction factors, as explained in additional detail below.

### 5.4.2 Method for Automatic Measuring and Sizing

As illustrated in the flow diagrams of FIGS. 26A-26D, one aspect of the present technology is a method for controlling a processor, such as processor 310, to measure user's facial features using two-dimensional or three-dimensional images and to recommend or select an appropriate facial interface size, such as from a group of standard sizes, based on the resultant measurements. The method may generally be characterized as including three or four different phases: a pre-capture phase 400, a capture phase 500, a post-capture image processing phase 600, and a comparison and output phase 700.

In some cases, the application for facial feature measuring and facial interface sizing may control a processor 310 to output a visual display that includes a reference feature on the display interface 320. The user may position the feature adjacent to their facial features, such as by movement of the camera. The processor may then capture and store one or more images of the facial features in association with the reference feature when certain conditions, such as alignment conditions are satisfied. This may be done with the assistance of a mirror 330. The mirror 330 reflects the displayed reference feature and the user's face to the camera. The application then controls the processor 310 to identify certain facial features within the images and measure distances therebetween. By image analysis processing a scaling factor may then be used to convert the facial feature measurements, which may be pixel counts, to standard facial interface measurement values based on the reference feature. Such values may be, for example, standardized unit of measure, such as a meter or an inch, and values expressed in such units suitable for interface sizing. Additional correction factors may be applied to the measurements. The facial feature measurements may be compared to data records that include measurement ranges corresponding to different interface sizes for particular interface forms. The recommended size may then be chosen and be output to the user/based on the comparison(s) as a recommendation. Such a process may be conveniently effected within the comfort of the user's own home, if the user so chooses. The application may perform this method within seconds. In one example, the application performs this method in real time. A manufacturer or supplier may arrange for the facial interface of the recommended size to be shipped to a user nominated address automatically.

### 5.5 Methods and Systems for Producing a Customised Patient Interface

Described below are systems and methods according to additional examples of the present technology, for production of a lattice structure of a patient interface or component thereof. The systems and methods below may be used together with the automatic sizing and personalisation examples above, or as alternatives. References to a patient interface that is customised, tailored, personalised, optimised etc. are to be understood to refer to a patient interface that has at least one component that is customised (e.g. a cushion 3130 by way of a customised lattice structure), even if some or all of the other components of the patient interface are not customised.

### 5.5.1 System Architecture

Examples of the system(s) outlined herein may include one or more computing devices with one or more processor(s) programmed or configured to perform the various functions described herein. While examples may describe certain information being stored and/or processing tasks being performed by a particular device, it will be appreciated that alternative embodiments are contemplated in which such information and/or processing tasks are shared.

FIG. 27 shows a schematic view of an exemplary system 100 that may be used to perform various aspects of the present technology as described herein. It will be appreciated that system 100 may receive data from, and send data to, external systems, and may control the operation of components outside of the system 100. The system 100 may generally include a customisation server 102 that manages the collection and processing of data relating to the design and production of a customised component for a patient interface 3000. The customisation server 102 has processing facilities represented by one or more processors 104, memory 106, and other components typically present in such computing devices. It should be appreciated that the server 102, processors 104, and memory 106 may take any suitable form known in the art, for example a “cloud-based” distributed server architecture or a dedicated server architecture. In the exemplary embodiment illustrated the memory 106 stores information accessible by processor 104, the information including instructions 108 that may be executed by the processor 104 and data 110 that may be retrieved, manipulated or stored by the processor 104. The memory 106 may be of any suitable means known in the art, capable of storing information in a manner accessible by the processor 104, including a computer-readable medium, or other medium that stores data that may be read with the aid of an electronic device.

The processor 104 may be any suitable device known to a person skilled in the art. Although the processor 104 and memory 106 are illustrated as being within a single unit, it should be appreciated that this is not intended to be limiting, and that the functionality of each as herein described may be performed by multiple processors and memories, that may or may not be remote from each other and other components of the system 100. The instructions 108 may include any set of instructions suitable for execution by the processor 104. For example, the instructions 108 may be stored as computer code on the computer-readable medium. The instructions may be stored in any suitable computer language or format. Data 110 may be retrieved, stored or modified by processor 104 in accordance with the instructions 110. The data 110 may also be formatted in any suitable computer readable format. The data 110 may also include a record 112 of control routines or algorithms for implementing aspects of the system 100.

Although the server 102 in FIG. 27 is shown only to include memory 106, the server 102 may further be capable of accessing other external memories, data stores, or databases (not shown). For example, information processed at the server 102 may be sent to an external data store (or database) to be stored, or may be accessed by the server 102 from the external data store (or database) for further processing. Additionally, the system 100 may include multiple such data stores and/or databases. In some cases, the data stores or databases may be separately accessible, such as each being accessible to a different server. In other cases, the data stores or databases described herein may not necessarily be separate, but may be stored together but as part of separate files, folders, columns of a table in a common file, etc.

The server 102 may communicate with an operator workstation 114 to provide an operator with access to various functions and information. Such communication may be performed via network 120. The network 120 may comprise various configurations and protocols including the Internet, intranets, virtual private networks, wide area networks, local networks, private networks using communication protocols proprietary to one or more companies, whether wired or wireless, or a combination thereof.

The server 102 performing one or more operations may include using artificial intelligence and/or machine learning algorithms. The server 102 may be configured to generate training datasets and/or employ trained datasets (by the server 102 or external to the server 102) to make certain decisions.

The exemplary system 100 includes one or more user devices 130 equipped to obtain data relating to shape and/or size of a user's face, head or features thereof, as will be described further below. By way of example, the user devices 130 may include a mobile computing device such as smart phone 130A or tablet computer 130B, or personal computing device such as a laptop or desktop computer 130C, each equipped with an image sensor such as a camera. While the present technology will be described herein as utilising image data obtained using a camera, alternative embodiments are contemplated in which other sensors are used to obtain the data relating to shape and/or size of a user's head or features thereof. For example, such sensors may include stereoscopic cameras for capturing three-dimensional images, or a light detector capable of detecting reflected light from a laser or strobing/structured light source.

The exemplary system 100 may include one or more manufacturing systems 140, configured to manufacture customised patient interfaces or components thereof. The manufacturing system 140 may include one or more manufacturing apparatus 142 configured to physically produce a component of a patient interface 3000. In some examples, the manufacturing apparatus 142 is a 3D printer, knitting machine, weaving machine, laser cutting machine or other additive manufacturing apparatus. In examples, the manufacturing system 140 may include multiple types of manufacturing apparatus 142 for manufacture of different components of a patient interface 3000. The manufacturing apparatus 142 may comprise one or more controllers 144 for control of the operative hardware 146 (e.g. knitting hardware or 3D printing hardware), and dedicated user interfaces for operator input/monitoring of the manufacturing apparatus 142. The manufacturing apparatus 142 may also communicate with other components of the manufacturing system, for example a manufacturing server 150 managing production of custom patient interfaces or components thereof in communication with the customisation server 102, and/or a manufacturing operator workstation 152.

In some examples, one or more of the manufacturing apparatus 142 is a laser cutter configured to cut out one or more components of the patient interface and/or modify produced one or more components (e.g., a component produced by another manufacturing apparatus 142). The laser cutter may provide flexibility to provide complex shapes with precision, repeatability, speed and/or automation. The laser cutter may also allow for components generated in large numbers to be customized with speed by modifying a length and/or a shape of the component based on the analysis results of the patient.

In some examples, one or more of the manufacturing apparatuses may be provided at a manufacturing plant, clinician's office, and/or at a patient's home. In some examples, a component may be produced at one location by one or more of the manufacturing apparatuses and then further modified by one or more of the manufacturing apparatuses at another location. In some examples, the one or more manufacturing apparatuses disposed at different location may receive instructions from the same manufacturing server 150, the same customisation server 102, and/or the same manufacturing operator workstation 152. In some examples, the one or more manufacturing apparatuses disposed at different location may report results of producing and/or modifying a component to the manufacturing server 150, the customisation server 102, and/or the manufacturing operator workstation 152.

In some examples, one or more of the devices in the exemplary system 100 may include communication circuitry configured to communicate with one or more other devices in the system 100 directly and/or via the network 120.

### 5.5.2 Methods for Customised Manufacture of Lattice Structure

As illustrated in the flow diagrams of FIGS. 28A-28E, one aspect of the present technology is a method 7000 of producing at least one customised component of a patient interface 3000 for treatment of sleep disordered breathing. The customised component may be, for example, a component of a seal-forming structure 3100 such as a cushion 3130 formed from a lattice structure. The customised component may be customised to an individual patient in one or more ways, such as in shape, size or by another property, for example a property described above in relation to personalisation and/or optimisation of a lattice structure.

Referring to FIG. 28A, examples of the method 7000 may generally be characterized as including three phases: a user data capture phase 7100, a specification phase 7200, and a production phase 7300.

### 5.5.2.1 User Data Capture Phase

In order to produce a patient interface that provides effective treatment and is comfortable for the user to wear, it is desirable to customize the patient interface, or at least components thereof, to conform with the size and/or shape of the user's head (and more particularly facial features). In order to provide such customization, it is often necessary to collect information about the size and/or shape of the user's head—in a number of cases including the user's facial features.

In examples of the present technology, the user data capture phase 7100 includes obtaining information representative of one or more landmark feature locations for a user's head. As used herein, the term “landmark” shall refer to particular points, a region or a feature on a human head associated with elements of the head, including facial features. The location of a landmark may be defined, for example, relative to other landmarks or a fixed reference point. Examples of head landmarks may include, without limitation: a subnasale, a sellion, a tragion, a posterior-most point of the patient's head, a superior-most point of the patient's head, a lateral-most point of the orbital margin, an inferior-most point of the orbital margin, the Frankfort horizontal plane, the sagittal plane and a coronal plane aligned with the tragion. Other examples of landmarks may be those features illustrated in any one of FIGS. 2B-2F.

### 5.5.2.1.1 Image Data Capture

In examples, obtaining the relevant information in the user capture phase 7100 may include capturing image data of at least a portion of a user's head at 7102 of FIG. 28B, and identifying the landmark feature locations based on the image data at 7104. By way of example, the image data may be captured using a camera of the smart phone 130A, tablet 130B, or computer 130C.

U.S. Patent Publication No. 2018/0117272, U.S. Patent Publication No. 2019/0167934, U.S. Pat. Nos. 7,827,038, 8,254,637, and 10,157,477 describe exemplary methods and systems for capturing data (e.g., image data) of at least a portion of a user's head, determining patient features, and/or fitting features of a mask to a patient, the contents of each of which are hereby incorporated herein by reference in their entirety. Other exemplary software tools for producing a three-dimensional model of a user's head, or portion thereof, may include: the “Capture” application available from Standard Cyborg, the “Scandy Pro” application available from Scandy, LLC; the “Beauty 3D” available from Guangzhou Zhimei Co., Ltd; the “Unre 3D FaceApp” available from UNRE AI LIMITED; and the “Bellus3D FaceApp” available from Bellus3D, Inc. Furthermore, any of the technology described elsewhere herein in relation to automatic sizing may be applied together with or as an alternative to the facial data acquisition technology described in this section.

In alternative examples, the relevant information may be obtained by a user or clinician performing a series of measurements on the user's head, and a record of these measurements created and entered into the system 100—i.e. circumventing the requirement to capture image data.

### 5.5.2.1.2 Landmark Feature Identification

In examples, identifying landmark features of the user at 7104 may be based on two-dimensional image data. An exemplary method and system for determining landmark features of a user, and locations of same, based on two-dimensional image data is described in U.S. Patent Publication No. 2018/0117272.

In examples, identifying landmark features based on the image data at 7104 may include producing a three-dimensional model of the user's face and/or head (at 7110 of FIG. 28C). The three-dimensional model may be analysed to identify landmark features of the user and determine locations of same at 7112. An exemplary method and system for identifying landmark features and locations of same from a three-dimensional model is described in U.S. Patent Publication No. 2019/0167934. As an example, the three-dimensional model may be generated based on data received from a 3D scanner, a stereo camera, and/or a plurality of images captured of the user's face and/or head from different positions and/or orientations of the capturing device and/or the patient.

In examples, local processing facilities at the point of capturing the image data (e.g. the smart phone 130A, tablet 130B, or computer 130C) may be used to identify the landmark features (including generation of the three-dimensional model in examples). In alternative examples, the image data may be communicated to remote processing facilities (e.g. customisation server 102) for further processing.

5.5.2.1.3 Relationships between Landmark Features

In some forms of the present technology, the method 7000 may include identifying relationships between landmark features. Such relationships may provide information regarding anthropometric measurements of the user to inform customisation of the patient interface, or component thereof, for the user. By way of example, a relationship between landmark features may include distance (i.e. spacing between the features), and relative angle.

In examples, identifying a relationship between landmark features may include determining distance between two or more of a subnasale, a sellion, a tragion, a posterior-most point of the patient's head, a superior-most point of the patient's head, a lateral-most point of the right orbital margin, a lateral-most point of the left orbital margin, an inferior-most point of the orbital margin, the Frankfort horizontal plane, and a coronal plane aligned with the tragion.

It will be appreciated that the landmark features (and their associated relationships) to be identified may be influenced by the design or configuration of the patient interface, or component thereof, to be manufactured-i.e. some landmark features will be relevant to certain designs or components, but not others. In examples, only select landmark features and their relationships may be assessed. In alternative examples, an entire set of landmark features from a list of possible landmark features that are capable of being identified may be assessed in order to allow use of the data set across a range of patient interfaces, or components thereof.

FIG. 29 shows a side view of a patient's head with a number of landmark feature spacings identified, described below. Each feature spacing is between a pair of landmark feature locations. Each of the spacings may be useful in determining the size and shape of the patient's head and locations of features thereof, for use in tailoring a cushion 3130 to the patient.

In examples a distance D1 between the subnasale and the coronal plane aligned with the tragion may be determined, the distance D1 being normal to said coronal plane. This landmark feature spacing may enable the spacing in the anterior-posterior axis between the patient's lip superior and the patient's ears to be accounted for in the design of a customised component for a patient interface 3000.

In examples a distance D2 in the sagittal plane between the subnasale and the tragion may be determined. The distance D2 may be a direct distance in the sagittal plane including both the vertical component and a horizontal component (e.g. a diagonal distance in the sagittal plane between the subnasale and vertically superior tragion). Together with the horizontal distance D1 between the subnasale and the tragion, this distance D2 may enable the height of the ear with respect to the lower periphery of the patient's nose to be taken into account in the design of a customised component for a patient interface 3000.

In examples a vertical distance D3 in the sagittal plane between the subnasale and the sellion may be determined. This distance D3 may enable the height of the patient's nose and/or the spacing between the lower periphery of the patient's nose and the patient's eyes to be accounted for in the design of customised component for a patient interface 3000. In particular, this spacing may be particularly useful in determining the shape and/or size of a customised cushion 3130 of seal forming structure 3100, for example.

In examples a distance D4 between the lateral-most point of the orbital margin and the coronal plane aligned with the tragion may be determined, the distance D4 being normal to said coronal plane. This spacing may enable the distance between the patient's ear and the patient's eye to be taken into account in the design of a customised component for a patient interface 3000.

In examples a vertical distance D5 between the subnasale and the superior-most point of the patient's head may be determined. This feature spacing may enable the height of the patient's head and the spacing between the lower periphery of the patient's nose and the top of the patient's head to be taken into account in the design of a customised component for a patient interface 3000. This feature spacing may be useful in determining the shape and/or size of a customised cushion 3130, for example.

In examples a vertical distance D6 between the superior-most point of the patient's head and the Frankfort horizontal plane may be determined. This feature spacing may enable the distance between top the patient's head and the patient's ear or lower orbital margin to be taken into account in the design of a customised component for a patient interface 3000. This distance may be useful in determining the shape and/or size of a customised cushion 3130, for example.

In examples a distance D7 between the rearmost point of the head and a coronal plane aligned with the tragion may be determined, the distance D7 being normal to said coronal plane. This feature spacing may enable the size of the patient's head and/or the distance between the patient's ear and the rear of the patient's head to be taken into account in the design of a customised component for a patient interface 3000.

The preceding relationships are given by example only, and are not intended to be limiting to all forms of the present technology.

### 5.5.2.2 Specification Phase

In the specification phase 7200, examples of the method 7000 include determining a set of manufacturing specifications for production of a patient interface, or one or more components thereof such as a cushion 3130 formed from a lattice structure or the lattice structure thereof, based on the one or more landmark feature locations and/or relationships between same.

In examples, such specifications are determined based on one or more performance requirements of the component. Examples of such performance requirements may include one or more of: stiffness, contact pressure, compliance, forces to be applied by or to the component, elasticity, dimensions (including size and relative angles of features of the component), tactile feel, breathability, heat dissipation, and/or positioning on the user's head. Such performance criteria may be influenced by one or more of: efficacy of delivery of the treatment (for example, sealing of the patient interface and/or reducing the likelihood of occlusion during use), user comfort (for example, the feel of the component to the touch, and relative positioning to avoid more sensitive areas of the user's head), and manufacturing considerations (for example, material costs and/or complexity of manufacture). It will be appreciated that the performance requirements for a component will be influenced by the one or more landmark feature locations and/or relationships between same, examples of which are described further below. In examples, the customised component specifications may be determined based in part on non-performance characteristics such as colour.

In examples, the performance requirements may be based on functional requirements which are not derived from the landmark feature locations and/or relationships between same, as described above.

In some examples the step of determining the at least one performance requirement comprises receiving and analysing facial movement data representing changes in shape and/or size of the patient's face during facial movement. In one form of the present technology the patient may be prompted to record themself making different facial expressions as part of acquiring facial movement data, which may be used in optimising the response of the lattice structure of the cushion 3130 to facial movement. For example, the cushion 3130 may be tuned to respond to the way a unique user's face tends to move.

FIGS. 30A and 30B show two facial expressions. In one example the patient may record or otherwise image or video capture themselves making a closed mouth expression as shown in FIG. 30A and then changing to an open mouth expression in FIG. 30B. Changes in the face size and/or shape between the two expressions may be analysed and used to optimise the response of the lattice structure 3130, for example to enable the cushion 3130 to maintain sufficient and preferably highly comfortable levels of contact pressure at the sealing surface during facial movement.

In some examples the patient may be prompted to record additional or alternative facial expressions to those shown in FIGS. 30A-30B, such as clenched jaw, relaxed jaw, partially open mouth, pursed lips, smiling, frowning, bared teeth, inflated cheeks expressions and/or in positions such as looking up, looking down, looking to the side, lying on a pillow looking upwards, side sleeping on a pillow, among others. In some examples the facial movement data may be acquired when the patient is talking.

In some examples the changes in locations and/or relative spacings of landmark features on the patient's face during facial movement may be analysed and manufacturing specifications may be produced at least partially on said changes.

The performance requirements and resulting manufacturing specifications will depend on the particular type or style of customised component to be produced.

In examples, the customised component may include a cushion 3130 or lattice structure thereof, as described herein. References to a production of a customised cushion 3130 are to be understood to be references to at least a lattice structure thereof, whether or not the lattice structure forms the entire completed cushion 3130 or not. The cushion 3130 may be customised to a particular patient by being formed in a particular shape and/or size, based on the landmark feature locations and/or relationships, that results in a comfortable and stable fit for that particular patient. Exemplary cushions 3130 are described above with reference to FIGS. 7-23F.

In examples, the performance requirements of one component of the patient interface may be influenced by properties or characteristics of another component. By way of example, for a customised cushion 3130 certain performance requirements may be determined in part by dimensions and/or configurations of a chassis portion 3210 or plenum chamber 3200 to which the cushion 3130 is to be attached.

In examples, the manufacturing specifications may comprise material specifications. A particular material, or blend of materials, may be selected based on a performance requirement such as stiffness, hardness, flexibility, compliance, or tactile feel. In some examples a material may be selected based on preferences of the patient for whom the customised component is being produced.

In examples, the manufacturing specifications may comprise construction technique specifications. For example, in a cushion 3130 comprising a lattice structure, a particular type/pattern of lattice structure (e.g. one of the example structures in FIGS. 23A-23F) may be selected based on performance requirements for the component. Where the lattice structure is formed by knitting then knitting stitch(es) may be specified.

In examples, determining a set of manufacturing specifications may comprise selecting a set of manufacturing specifications from a plurality of pre-existing sets of manufacturing specifications. In examples, determining a set of manufacturing specifications may comprise selecting a plurality of manufacturing specifications to form the set of manufacturing specifications from a plurality of pre-existing manufacturing specifications. Selection of pre-existing manufacturing specifications may be based on similarities between the one or more landmark feature locations and/or relationships determined for the user, and those associated with the pre-existing manufacturing specification.

Identifying the landmark features and/or their location (e.g., at 7104 and/or 7112), identifying relationships between the landmark features, determining functional requirements (e.g., for a patient interface and/or one or more components thereof) (e.g., at 7202), and/or determining manufacturing specifications (e.g., at 7204) may include using artificial intelligence and/or machine learning algorithms.

For example, a trained dataset may be used to identify the landmark features and/or their location. In some examples, the captured image data and/or the three dimensional models used to identify the landmark features may be used to train datasets. In another example, a trained data set may be used to identify manufacturing specifications based on the landmark features, their locations, and/or functional requirements.

### 5.5.2.3 Producing the Customised Component

In examples, producing the patient interface or component thereof (e.g. cushion 3130) based on the set of manufacturing specifications at 7300 comprises producing manufacturing machine programming instructions for production of the patient interface or component thereof based on the set of manufacturing specifications at 7302 (see FIG. 28E). The manufacturing machines 142 are programmed with the manufacturing machine programming instructions at 7304, and are operated according to the manufacturing machine programming instructions to produce the patient interface or component thereof at 7306.

In some examples, producing the customised component at 7300 comprises additive manufacturing (for example, 3D printing) of the customised component. The manufacturing machines 142 may comprise a 3D printer to print the customised component, for example the cushion 3130 or lattice structure thereof. The manufacturing machines 142 may comprise a laser cutter to cut-out and/or modify a customised component, for example a cushion 3130 formed from foam and having laser cut holes to form it into a lattice structure.

### 5.5.2.3.1 Producing Manufacturing Machine Programming Instructions

In examples, the manufacturing machine programming instructions for production of the patient interface or component thereof (e.g. cushion 3130) may be generated automatically based on the set of manufacturing specifications. In examples, the manufacturing machine programming instructions may be generated from a model of the patient interface or component embodying the set of manufacturing specifications. Software tools are known for producing manufacturing machine programming instructions from two-dimensional and three-dimensional models. Some aspects of the programming instructions may be determined automatically.

In examples, producing manufacturing machine programming instructions for production of the patient interface or component thereof based on the set of manufacturing specifications at 7302 comprises generating a map representing the one or more manufacturing specifications at 7310 (see FIG. 28F). In such examples, producing the manufacturing machine programming instructions at 7302 comprises generating the instructions based on the map representing the manufacturing specifications at 7312.

In an example, the map may comprise a two-dimensional model of the patient interface or component thereof, e.g. one or more two-dimensional images. In examples, details of the manufacturing specifications may be supplied by visually coding the model—i.e. certain manufacturing specifications may be obtained by visual recognition of characteristics of the map. In an example, the map may comprise a three-dimensional model of the patient interface or component thereof. In such an example, details of the manufacturing specifications may be encoded into the three-dimensional model.

In examples the map may be generated at a first processing facility, for example customisation server 102, and communicated to the appropriate manufacturer system 140 for generation of the manufacturing machine programming instructions.

In other examples, the generation of the map and the manufacturing machine programming instructions may be performed at a single processing facility, for example by generating the map using a first software application, and generating the manufacturing machine programming instructions using a second software application.

In examples, the map may be converted into a model from which the manufacturing machine programming instructions may be generated. In an alternative example, the manufacturing specifications may be embodied in a map configured to be converted directly into the manufacturing machine programming instructions. In examples, the set of manufacturing specifications may be converted into the manufacturing machine programming instructions without an intermediary model or map being generated.

In examples, the set of manufacturing specifications may be used to modify a pre-existing template from which the manufacturing machine programming instructions are generated. Such templates may have predefined baseline rules associated with them, for example relating to manufacturing constraints, or universal performance requirements for a particular component design. In exemplary embodiments, such templates may include predefined regions of the component design, wherein the manufacturing specifications are used to modify parameters of each predefined region.

### 5.5.2.4 Distribution of Customised Component

In examples, following production of the customised patient interface or component thereof, an automated distribution system may be used to manage delivery to the user. In examples, the customised patient interface or component thereof (e.g. cushion 3130) may be delivered directly to the user from the facilities of the manufacturing system 140, or to a designated collection point or address.

In examples in which multiple components are to be produced, or at least supplied together with at least one customised component, an assembly phase may be performed. In examples, assembly may be performed by the vendor of the patient interface. Where the manufacturer of the customised component is a third party, the customised component may be delivered to a facility of the vendor for assembly with other components prior to delivery to the user.

### 5.5.2.5 Matching of User to Existing Products

According to an aspect of the present technology, user-specific data (e.g. measurements obtained from the user, or user profile information) may be used to select a patient interface component from a group of pre-existing component configurations having associated manufacturing specifications and programming instructions. For example, the selection may be based on a comparison between user-specific data and a data record relating to information associated with the pre-existing component configurations.

In examples, the pre-existing component configurations may be developed based on one or more sets of data representative of landmark features of heads representative of a user base. For example, a set of data may comprise a model of a human head having characteristics associated with profile categories such as gender, age, or build. Such models may be trained, for example using artificial intelligence and/or machine learning algorithms. Manufacturing specifications may be developed based on analysis of such representative models, and programming instructions generated from same.

5.5.2.6 Using Feedback to Modify Specification and/or Update Models

According to an aspect of the present technology, feedback from the user, clinician and/or manufacturing operator may be used to update parameters and/or models used to perform one or more of the above discussed operations (e.g., identifying the landmark features and/or their location, identifying relationships between landmark features, determining functional requirements, and/or determining manufacturing specifications). The feedback may be received via a user interface displayed on the RPT device, the user devices 130, the operator workstation 114, and/or the manufacturing operator workstation 152.

The user may provide feedback after receiving the customized patient interface. The use may input information indicating how well the patient interface fits when the patient interface is first used, after a predetermined period of time (e.g., after receiving or starting to use the patient interface), and/or after a predetermined amount of use. The user may be asked predefined questions about different aspects of the patient interface and/or asked to rate different features of the mask. The clinician may input feedback received from the user and/or feedback based on observing the user using the patient interface. The manufacturing operator may provide feedback based on the customized patient interfaces being produced by the manufacturing apparatus 142. For example, the manufacturing operator may inspect the manufactured patient interface and input defects in the patient interface caused by the manufacturing process.

The feedback from the user, clinician and/or manufacturing operator may be used to modify manufacturing specifications and/or update models used (e.g., by artificial intelligence and/or machine learning algorithms) to identify the landmark features and/or their location, to identify relationships between landmark features and/or to identify manufacturing specifications.

### 5.6 RPT Device

An RPT device 4000 in accordance with one aspect of the present technology comprises mechanical, pneumatic, and/or electrical components and is configured to execute one or more algorithms, such as any of the methods, in whole or in part, described herein. The RPT device 4000 may be configured to generate a flow of air for delivery to a patient's airways, such as to treat one or more of the respiratory conditions described elsewhere in the present document.

In one form, the RPT device 4000 is constructed and arranged to be capable of delivering a flow of air in a range of −20 L/min to +150 L/min while maintaining a positive pressure of at least 6 cmH2O, or at least 10 cmH2O, or at least 20 cmH2O

The RPT device may have an external housing 4010, formed in two parts, an upper portion 4012 and a lower portion 4014. Furthermore, the external housing 4010 may include one or more panel(s) 4015. The RPT device 4000 comprises a chassis 4016 that supports one or more internal components of the RPT device 4000. The RPT device 4000 may include a handle 4018.

The pneumatic path of the RPT device 4000 may comprise one or more air path items, e.g., an inlet air filter 4112, an inlet muffler 4122, a pressure generator 4140 capable of supplying air at positive pressure (e.g., a blower 4142), an outlet muffler 4124 and one or more transducers 4270, such as pressure sensors and flow rate sensors.

One or more of the air path items may be located within a removable unitary structure which will be referred to as a pneumatic block 4020. The pneumatic block 4020 may be located within the external housing 4010. In one form a pneumatic block 4020 is supported by, or formed as part of the chassis 4016.

The RPT device 4000 may have an electrical power supply 4210, one or more input devices 4220, a central controller, a therapy device controller, a pressure generator 4140, one or more protection circuits, memory, transducers 4270, a data communication interface and one or more output devices. Electrical components 4200 may be mounted on a single Printed Circuit Board Assembly (PCBA) 4202. In an alternative form, the RPT device 4000 may include more than one PCBA 4202.

### 5.6.1 Rpt Device Mechanical & Pneumatic Components

An RPT device may comprise one or more of the following components in an integral unit. In an alternative form, one or more of the following components may be located as respective separate units.

5.6.1.1 Air filter(s)

An RPT device in accordance with one form of the present technology may include an air filter 4110, or a plurality of air filters 4110.

In one form, an inlet air filter 4112 is located at the beginning of the pneumatic path upstream of a pressure generator 4140.

In one form, an outlet air filter 4114, for example an antibacterial filter, is located between an outlet of the pneumatic block 4020 and a patient interface 3000.

### 5.6.1.2 Muffler(s)

An RPT device in accordance with one form of the present technology may include a muffler 4120, or a plurality of mufflers 4120.

In one form of the present technology, an inlet muffler 4122 is located in the pneumatic path upstream of a pressure generator 4140.

In one form of the present technology, an outlet muffler 4124 is located in the pneumatic path between the pressure generator 4140 and a patient interface 3000.

### 5.6.1.3 Pressure Generator

In one form of the present technology, a pressure generator 4140 for producing a flow, or a supply, of air at positive pressure is a controllable blower 4142. For example, the blower 4142 may include a brushless DC motor 4144 with one or more impellers. The impellers may be located in a volute. The blower may be capable of delivering a supply of air, for example at a rate of up to about 120 litres/minute, at a positive pressure in a range from about 4 cmH2O to about 20 cmH2O, or in other forms up to about 30 cmH2O when delivering respiratory pressure therapy. The blower may be as described in any one of the following patents or patent applications the contents of which are incorporated herein by reference in their entirety: U.S. Pat. Nos. 7,866,944; 8,638,014; 8,636,479; and PCT Patent Application Publication No. WO 2013/020167.

The pressure generator 4140 may be under the control of the therapy device controller.

In other forms, a pressure generator 4140 may be a piston-driven pump, a pressure regulator connected to a high pressure source (e.g. compressed air reservoir), or a bellows.

### 5.6.1.4 Anti-Spill Back Valve

In one form of the present technology, an anti-spill back valve 4160 is located between the humidifier 5000 and the pneumatic block 4020. The anti-spill back valve is constructed and arranged to reduce the risk that water will flow upstream from the humidifier 5000, for example to the motor 4144.

### 5.6.2 RPT Device Algorithms

As mentioned above, in some forms of the present technology, a central controller of the RPT device 4000 may be configured to implement one or more algorithms expressed as computer programs stored in a non-transitory computer readable storage medium, such as memory. The algorithms are generally grouped into groups referred to as modules.

In other forms of the present technology, some portion or all of the algorithms may be implemented by a controller of an external device such as the local external device or the remote external device. In such forms, data representing the input signals and/or intermediate algorithm outputs necessary for the portion of the algorithms to be executed at the external device may be communicated to the external device via the local external communication network or the remote external communication network. In such forms, the portion of the algorithms to be executed at the external device may be expressed as computer programs, such as with processor control instructions to be executed by one or more processor(s), stored in a non-transitory computer readable storage medium accessible to the controller of the external device. Such programs configure the controller of the external device to execute the portion of the algorithms.

In such forms, the therapy parameters generated by the external device via the therapy engine module (if such forms part of the portion of the algorithms executed by the external device) may be communicated to the central controller to be passed to the therapy control module.

### 5.7 Air Circuit

An air circuit 4170 in accordance with an aspect of the present technology is a conduit or a tube constructed and arranged to allow, in use, a flow of air to travel between two components such as RPT device 4000 and the patient interface 3000.

In particular, the air circuit 4170 may be in fluid connection with the outlet of the pneumatic block 4020 and the patient interface. The air circuit may be referred to as an air delivery tube. In some cases there may be separate limbs of the circuit for inhalation and exhalation. In other cases a single limb is used.

### 5.8 Humidifier

5.8.1 Humidifier overview

In one form of the present technology there is provided a humidifier 5000 (e.g. as shown in FIG. 5A) to change the absolute humidity of air or gas for delivery to a patient relative to ambient air. Typically, the humidifier 5000 is used to increase the absolute humidity and increase the temperature of the flow of air (relative to ambient air) before delivery to the patient's airways.

The humidifier 5000 may comprise a humidifier reservoir 5110, a humidifier inlet 5002 to receive a flow of air, and a humidifier outlet 5004 to deliver a humidified flow of air. In some forms, as shown in FIG. 5A and FIG. 5B, an inlet and an outlet of the humidifier reservoir 5110 may be the humidifier inlet 5002 and the humidifier outlet 5004 respectively. The humidifier 5000 may further comprise a humidifier base 5006, which may be adapted to receive the humidifier reservoir 5110 and comprise a heating element 5240.

### 5.8.2 Humidifier Components

### 5.8.2.1 Water Reservoir

According to one arrangement, the humidifier 5000 may comprise a water reservoir 5110 configured to hold, or retain, a volume of liquid (e.g. water) to be evaporated for humidification of the flow of air. The water reservoir 5110 may be configured to hold a predetermined maximum volume of water in order to provide adequate humidification for at least the duration of a respiratory therapy session, such as one evening of sleep. Typically, the reservoir 5110 is configured to hold several hundred millilitres of water, e.g. 300 millilitres (ml), 325 ml, 350 ml or 400 ml. In other forms, the humidifier 5000 may be configured to receive a supply of water from an external water source such as a building's water supply system.

According to one aspect, the water reservoir 5110 is configured to add humidity to a flow of air from the RPT device 4000 as the flow of air travels therethrough. In one form, the water reservoir 5110 may be configured to encourage the flow of air to travel in a tortuous path through the reservoir 5110 while in contact with the volume of water therein.

According to one form, the reservoir 5110 may be removable from the humidifier 5000, for example in a lateral direction as shown in FIG. 5A and FIG. 5B.

The reservoir 5110 may also be configured to discourage egress of liquid therefrom, such as when the reservoir 5110 is displaced and/or rotated from its normal, working orientation, such as through any apertures and/or in between its sub-components. As the flow of air to be humidified by the humidifier 5000 is typically pressurised, the reservoir 5110 may also be configured to prevent losses in pneumatic pressure through leak and/or flow impedance.

### 5.8.2.2 Conductive Portion

According to one arrangement, the reservoir 5110 comprises a conductive portion 5120 configured to allow efficient transfer of heat from the heating element 5240 to the volume of liquid in the reservoir 5110. In one form, the conductive portion 5120 may be arranged as a plate, although other shapes may also be suitable. All or a part of the conductive portion 5120 may be made of a thermally conductive material such as aluminium (e.g. approximately 2 mm thick, such as 1 mm, 1.5 mm, 2.5 mm or 3 mm), another heat conducting metal or some plastics. In some cases, suitable heat conductivity may be achieved with less conductive materials of suitable geometry.

### 5.8.2.3 Humidifier Reservoir Dock

In one form, the humidifier 5000 may comprise a humidifier reservoir dock 5130 (as shown in FIG. 5B) configured to receive the humidifier reservoir 5110. In some arrangements, the humidifier reservoir dock 5130 may comprise a locking feature such as a locking lever 5135 configured to retain the reservoir 5110 in the humidifier reservoir dock 5130.

### 5.8.2.4 Water Level Indicator

The humidifier reservoir 5110 may comprise a water level indicator 5150 as shown in FIG. 5A-5B. In some forms, the water level indicator 5150 may provide one or more indications to a user such as the patient 1000 or a care giver regarding a quantity of the volume of water in the humidifier reservoir 5110. The one or more indications provided by the water level indicator 5150 may include an indication of a maximum, predetermined volume of water, any portions thereof, such as 25%, 50% or 75% or volumes such as 200 ml, 300 ml or 400 ml.

### 5.8.2.5 Heating Element

A heating element 5240 may be provided to the humidifier 5000 in some cases to provide a heat input to one or more of the volume of water in the humidifier reservoir 5110 and/or to the flow of air. The heating element 5240 may comprise a heat generating component such as an electrically resistive heating track. One suitable example of a heating element 5240 is a layered heating element such as one described in the PCT Patent Application Publication No. WO 2012/171072, which is incorporated herewith by reference in its entirety.

In some forms, the heating element 5240 may be provided in the humidifier base 5006 where heat may be provided to the humidifier reservoir 5110 primarily by conduction as shown in FIG. 5B.

### 5.9 Breathing Waveforms

FIG. 6A shows a model typical breath waveform of a person while sleeping. The horizontal axis is time, and the vertical axis is respiratory flow rate.
While the parameter values may vary, a typical breath may have the following approximate values: tidal volume Vt 0.5 L, inhalation time Ti 1.6s, peak inspiratory flow rate Qpeak 0.4 L/s, exhalation time Te 2.4s, peak expiratory flow rate Qpeak −0.5 L/s. The total duration of the breath, Ttot, is about 4s. The person typically breathes at a rate of about 15 breaths per minute (BPM), with Ventilation Vent about 7.5 L/min. A typical duty cycle, the ratio of Ti to Ttot, is about 40%.

### 5.10 Glossary

For the purposes of the present technology disclosure, in certain forms of the present technology, one or more of the following definitions may apply. In other forms of the present technology, alternative definitions may apply.

### 5.10.1 General

Air: In certain forms of the present technology, air may be taken to mean atmospheric air, and in other forms of the present technology air may be taken to mean some other combination of breathable gases, e.g. oxygen enriched air.

Ambient: In certain forms of the present technology, the term ambient will be taken to mean (i) external of the treatment system or patient, and (ii) immediately surrounding the treatment system or patient.

For example, ambient humidity with respect to a humidifier may be the humidity of air immediately surrounding the humidifier, e.g. the humidity in the room where a patient is sleeping. Such ambient humidity may be different to the humidity outside the room where a patient is sleeping.

In another example, ambient pressure may be the pressure immediately surrounding or external to the body.

In certain forms, ambient (e.g., acoustic) noise may be considered to be the background noise level in the room where a patient is located, other than for example, noise generated by an RPT device or emanating from a mask or patient interface. Ambient noise may be generated by sources outside the room.

Automatic Positive Airway Pressure (APAP) therapy: CPAP therapy in which the treatment pressure is automatically adjustable, e.g. from breath to breath, between minimum and maximum limits, depending on the presence or absence of indications of SDB events.

Continuous Positive Airway Pressure (CPAP) therapy: Respiratory pressure therapy in which the treatment pressure is approximately constant through a respiratory cycle of a patient. In some forms, the pressure at the entrance to the airways will be slightly higher during exhalation, and slightly lower during inhalation. In some forms, the pressure will vary between different respiratory cycles of the patient, for example, being increased in response to detection of indications of partial upper airway obstruction, and decreased in the absence of indications of partial upper airway obstruction.

Flow rate: The volume (or mass) of air delivered per unit time. Flow rate may refer to an instantaneous quantity. In some cases, a reference to flow rate will be a reference to a scalar quantity, namely a quantity having magnitude only. In other cases, a reference to flow rate will be a reference to a vector quantity, namely a quantity having both magnitude and direction. Flow rate may be given the symbol Q. ‘Flow rate’is sometimes shortened to simply ‘flow’or ‘airflow’.

In the example of patient respiration, a flow rate may be nominally positive for the inspiratory portion of a breathing cycle of a patient, and hence negative for the expiratory portion of the breathing cycle of a patient. Device flow rate, Qd, is the flow rate of air leaving the RPT device. Total flow rate, Qt, is the flow rate of air and any supplementary gas reaching the patient interface via the air circuit. Vent flow rate, Qv, is the flow rate of air leaving a vent to allow washout of exhaled gases. Leak flow rate, Ql, is the flow rate of leak from a patient interface system or elsewhere. Respiratory flow rate, Qr, is the flow rate of air that is received into the patient's respiratory system.

Flow therapy: Respiratory therapy comprising the delivery of a flow of air to an entrance to the airways at a controlled flow rate referred to as the treatment flow rate that is typically positive throughout the patient's breathing cycle.

Humidifier: The word humidifier will be taken to mean a humidifying apparatus constructed and arranged, or configured with a physical structure to be capable of providing a therapeutically beneficial amount of water (H₂O) vapour to a flow of air to ameliorate a medical respiratory condition of a patient.

Leak: The word leak will be taken to be an unintended flow of air. In one example, leak may occur as the result of an incomplete seal between a mask and a patient's face. In another example leak may occur in a swivel elbow to the ambient.

Noise, conducted (acoustic): Conducted noise in the present document refers to noise which is carried to the patient by the pneumatic path, such as the air circuit and the patient interface as well as the air therein. In one form, conducted noise may be quantified by measuring sound pressure levels at the end of an air circuit.

Noise, radiated (acoustic): Radiated noise in the present document refers to noise which is carried to the patient by the ambient air. In one form, radiated noise may be quantified by measuring sound power/pressure levels of the object in question according to ISO 3744.

Noise, vent (acoustic): Vent noise in the present document refers to noise which is generated by the flow of air through any vents such as vent holes of the patient interface.

Oxygen enriched air: Air with a concentration of oxygen greater than that of atmospheric air (21%), for example at least about 50% oxygen, at least about 60% oxygen, at least about 70% oxygen, at least about 80% oxygen, at least about 90% oxygen, at least about 95% oxygen, at least about 98% oxygen, or at least about 99% oxygen. “Oxygen enriched air” is sometimes shortened to “oxygen”.

Medical Oxygen: Medical oxygen is defined as oxygen enriched air with an oxygen concentration of 80% or greater.

Patient: A person, whether or not they are suffering from a respiratory condition.

Pressure: Force per unit area. Pressure may be expressed in a range of units, including cmH₂O, g-f/cm² and hectopascal. 1 cmH₂O is equal to 1 g-f/cm² and is approximately 0.98 hectopascal (1 hectopascal=100 Pa=100 N/m²=1 millibar ~0.001 atm). In this specification, unless otherwise stated, pressure is given in units of cmH₂O.

The pressure in the patient interface is given the symbol Pm, while the treatment pressure, which represents a target value to be achieved by the interface pressure Pm at the current instant of time, is given the symbol Pt.

Respiratory Pressure Therapy: The application of a supply of air to an entrance to the airways at a treatment pressure that is typically positive with respect to atmosphere.

Ventilator: A mechanical device that provides pressure support to a patient to perform some or all of the work of breathing.

### 5.10.1.1 Materials

Silicone or Silicone Elastomer: A synthetic rubber. In this specification, a reference to silicone is a reference to liquid silicone rubber (LSR) or a compression moulded silicone rubber (CMSR). One form of commercially available LSR is SILASTIC (included in the range of products sold under this trademark), manufactured by Dow Corning. Another manufacturer of LSR is Wacker. Unless otherwise specified to the contrary, an exemplary form of LSR has a Shore A (or Type A) indentation hardness in the range of about 35 to about 45 as measured using ASTM D2240.

Polycarbonate: a thermoplastic polymer of Bisphenol-A Carbonate.

### 5.10.1.2 Mechanical Properties

Resilience: Ability of a material to absorb energy when deformed elastically and to release the energy upon unloading.

Resilient: Will release substantially all of the energy when unloaded. Includes e.g. certain silicones, and thermoplastic elastomers.

Hardness: The ability of a material per se to resist deformation (e.g. described by a Young's Modulus, or an indentation hardness scale measured on a standardised sample size).
‘Soft’ materials may include silicone or thermo-plastic elastomer (TPE), and may, e.g. readily deform under finger pressure.
‘Hard’ materials may include polycarbonate, polypropylene, steel or aluminium, and may not e.g. readily deform under finger pressure.

Stiffness (or rigidity) of a structure or component: The ability of the structure or component to resist deformation in response to an applied load. The load may be a force or a moment, e.g. compression, tension, bending or torsion. The structure or component may offer different resistances in different directions. The inverse of stiffness is flexibility.

Floppy structure or component: A structure or component that will change shape, e.g. bend, when caused to support its own weight, within a relatively short period of time such as 1 second.

Rigid structure or component: A structure or component that will not substantially change shape when subject to the loads typically encountered in use. An example of such a use may be setting up and maintaining a patient interface in sealing relationship with an entrance to a patient's airways, e.g. at a load of approximately 20 to 30 cmH2O pressure.

As an example, an I-beam may comprise a different bending stiffness (resistance to a bending load) in a first direction in comparison to a second, orthogonal direction. In another example, a structure or component may be floppy in a first direction and rigid in a second direction.

### 5.10.2 Respiratory Cycle

Apnea: According to some definitions, an apnea is said to have occurred when flow falls below a predetermined threshold for a duration, e.g. 10 seconds. An obstructive apnea will be said to have occurred when, despite patient effort, some obstruction of the airway does not allow air to flow. A central apnea will be said to have occurred when an apnea is detected that is due to a reduction in breathing effort, or the absence of breathing effort, despite the airway being patent. A mixed apnea occurs when a reduction or absence of breathing effort coincides with an obstructed airway.

Expiratory portion of a breathing cycle: The period from the start of expiratory flow to the start of inspiratory flow.

Hypopnea: According to some definitions, a hypopnea is taken to be a reduction in flow, but not a cessation of flow. In one form, a hypopnea may be said to have occurred when there is a reduction in flow below a threshold rate for a duration. A central hypopnea will be said to have occurred when a hypopnea is detected that is due to a reduction in breathing effort. In one form in adults, either of the following may be regarded as being hypopneas:
(3272) a 30% reduction in patient breathing for at least 10 seconds plus an associated 4% desaturation; or
(ii) a reduction in patient breathing (but less than 50%) for at least 10 seconds, with an associated desaturation of at least 3% or an arousal.

Hyperpnea: An increase in flow to a level higher than normal.

Inspiratory portion of a breathing cycle: The period from the start of inspiratory flow to the start of expiratory flow will be taken to be the inspiratory portion of a breathing cycle.

### 5.10.3 Anatomy

### 5.10.3.1 Anatomy of the Face

Ala: the external outer wall or “wing” of each nostril (plural: alar)

Alare: The most lateral point on the nasal ala.

Alar curvature (or alar crest) point: The most posterior point in the curved base line of each ala, found in the crease formed by the union of the ala with the cheek.

Auricle: The whole external visible part of the ear.

(nose) Bony framework: The bony framework of the nose comprises the nasal bones, the frontal process of the maxillae and the nasal part of the frontal bone.

(nose) Cartilaginous framework: The cartilaginous framework of the nose comprises the septal, lateral, major and minor cartilages.

Columella: the strip of skin that separates the nares and which runs from the pronasale to the upper lip.

Columella angle: The angle between the line drawn through the midpoint of the nostril aperture and a line drawn perpendicular to the Frankfort horizontal while intersecting subnasale.

Frankfort horizontal plane: A line extending from the most inferior point of the orbital margin to the left tragion. The tragion is the deepest point in the notch superior to the tragus of the auricle.

Glabella: Located on the soft tissue, the most prominent point in the midsagittal plane of the forehead.

Lateral nasal cartilage: A generally triangular plate of cartilage. Its superior margin is attached to the nasal bone and frontal process of the maxilla, and its inferior margin is connected to the greater alar cartilage.

Greater alar cartilage: A plate of cartilage lying below the lateral nasal cartilage. It is curved around the anterior part of the naris. Its posterior end is connected to the frontal process of the maxilla by a tough fibrous membrane containing three or four minor cartilages of the ala.

Nares (Nostrils): Approximately ellipsoidal apertures forming the entrance to the nasal cavity. The singular form of nares is naris (nostril). The nares are separated by the nasal septum.

Naso-labial sulcus or Naso-labial fold: The skin fold or groove that runs from each side of the nose to the corners of the mouth, separating the cheeks from the upper lip.

Naso-labial angle: The angle between the columella and the upper lip, while intersecting subnasale.

Otobasion inferior: The lowest point of attachment of the auricle to the skin of the face.

Otobasion superior: The highest point of attachment of the auricle to the skin of the face.

Pronasale: the most protruded point or tip of the nose, which can be identified in lateral view of the rest of the portion of the head.

Philtrum: the midline groove that runs from lower border of the nasal septum to the top of the lip in the upper lip region.

Pogonion: Located on the soft tissue, the most anterior midpoint of the chin.

Ridge (nasal): The nasal ridge is the midline prominence of the nose, extending from the Sellion to the Pronasale.

Sagittal plane: A vertical plane that passes from anterior (front) to posterior (rear). The midsagittal plane is a sagittal plane that divides the body into right and left halves.

Sellion: Located on the soft tissue, the most concave point overlying the area of the frontonasal suture.

Septal cartilage (nasal): The nasal septal cartilage forms part of the septum and divides the front part of the nasal cavity.

Subalare: The point at the lower margin of the alar base, where the alar base joins with the skin of the superior (upper) lip.

Subnasal point: Located on the soft tissue, the point at which the columella merges with the upper lip in the midsagittal plane.

Supramenton: The point of greatest concavity in the midline of the lower lip between labrale inferius and soft tissue pogonion

### 5.10.3.2 Anatomy of the Skull

Frontal bone: The frontal bone includes a large vertical portion, the squama frontalis, corresponding to the region known as the forehead.

Mandible: The mandible forms the lower jaw. The mental protuberance is the bony protuberance of the jaw that forms the chin.

Maxilla: The maxilla forms the upper jaw and is located above the mandible and below the orbits. The frontal process of the maxilla projects upwards by the side of the nose, and forms part of its lateral boundary.

Nasal bones: The nasal bones are two small oblong bones, varying in size and form in different individuals; they are placed side by side at the middle and upper part of the face, and form, by their junction, the “bridge” of the nose.

Nasion: The intersection of the frontal bone and the two nasal bones, a depressed area directly between the eyes and superior to the bridge of the nose.

Occipital bone: The occipital bone is situated at the back and lower part of the cranium. It includes an oval aperture, the foramen magnum, through which the cranial cavity communicates with the vertebral canal. The curved plate behind the foramen magnum is the squama occipitalis.

Orbit: The bony cavity in the skull to contain the eyeball.

Parietal bones: The parietal bones are the bones that, when joined together, form the roof and sides of the cranium.

Temporal bones: The temporal bones are situated on the bases and sides of the skull, and support that part of the face known as the temple.

Zygomatic bones: The face includes two zygomatic bones, located in the upper and lateral parts of the face and forming the prominence of the cheek.

### 5.10.3.3 Anatomy of the Respiratory System

Diaphragm: A sheet of muscle that extends across the bottom of the rib cage. The diaphragm separates the thoracic cavity, containing the heart, lungs and ribs, from the abdominal cavity. As the diaphragm contracts the volume of the thoracic cavity increases and air is drawn into the lungs.

Larynx: The larynx, or voice box houses the vocal folds and connects the inferior part of the pharynx (hypopharynx) with the trachea.

Lungs: The organs of respiration in humans. The conducting zone of the lungs contains the trachea, the bronchi, the bronchioles, and the terminal bronchioles. The respiratory zone contains the respiratory bronchioles, the alveolar ducts, and the alveoli.

Nasal cavity: The nasal cavity (or nasal fossa) is a large air filled space above and behind the nose in the middle of the face. The nasal cavity is divided in two by a vertical fin called the nasal septum. On the sides of the nasal cavity are three horizontal outgrowths called nasal conchae (singular “concha”) or turbinates. To the front of the nasal cavity is the nose, while the back blends, via the choanae, into the nasopharynx.

Pharynx: The part of the throat situated immediately inferior to (below) the nasal cavity, and superior to the oesophagus and larynx. The pharynx is conventionally divided into three sections: the nasopharynx (epipharynx) (the nasal part of the pharynx), the oropharynx (mesopharynx) (the oral part of the pharynx), and the laryngopharynx (hypopharynx).

### 5.10.4 Patient Interface

Anti-asphyxia valve (AAV): The component or sub-assembly of a mask system that, by opening to atmosphere in a failsafe manner, reduces the risk of excessive CO2 rebreathing by a patient.

Elbow: An elbow is an example of a structure that directs an axis of flow of air travelling therethrough to change direction through an angle. In one form, the angle may be approximately 90 degrees. In another form, the angle may be more, or less than 90 degrees. The elbow may have an approximately circular cross-section. In another form the elbow may have an oval or a rectangular cross-section. In certain forms an elbow may be rotatable with respect to a mating component, e.g. about 360 degrees. In certain forms an elbow may be removable from a mating component, e.g. via a snap connection. In certain forms, an elbow may be assembled to a mating component via a one-time snap during manufacture, but not removable by a patient.

Frame: Frame will be taken to mean a mask structure that bears the load of tension between two or more points of connection with a headgear. A mask frame may be a non-airtight load bearing structure in the mask. However, some forms of mask frame may also be air-tight.

Headgear: Headgear will be taken to mean a form of positioning and stabilizing structure designed for use on a head. For example the headgear may comprise a collection of one or more struts, ties and stiffeners configured to locate and retain a patient interface in position on a patient's face for delivery of respiratory therapy. Some ties are formed of a soft, flexible, elastic material such as a laminated composite of foam and fabric.

Membrane: Membrane will be taken to mean a typically thin element that has, preferably, substantially no resistance to bending, but has resistance to being stretched.

Plenum chamber: a mask plenum chamber will be taken to mean a portion of a patient interface having walls at least partially enclosing a volume of space, the volume having air therein pressurised above atmospheric pressure in use. A shell may form part of the walls of a mask plenum chamber.

Seal: May be a noun form (“a seal”) which refers to a structure, or a verb form (“to seal”) which refers to the effect. Two elements may be constructed and/or arranged to ‘seal’ or to effect ‘sealing’ therebetween without requiring a separate ‘seal’element per se.

Shell: A shell will be taken to mean a curved, relatively thin structure having bending, tensile and compressive stiffness. For example, a curved structural wall of a mask may be a shell. In some forms, a shell may be faceted. In some forms a shell may be airtight. In some forms a shell may not be airtight.

Stiffener: A stiffener will be taken to mean a structural component designed to increase the bending resistance of another component in at least one direction.

Strut: A strut will be taken to be a structural component designed to increase the compression resistance of another component in at least one direction.

Swivel (noun): A subassembly of components configured to rotate about a common axis, preferably independently, preferably under low torque. In one form, the swivel may be constructed to rotate through an angle of at least 360 degrees. In another form, the swivel may be constructed to rotate through an angle less than 360 degrees. When used in the context of an air delivery conduit, the sub-assembly of components preferably comprises a matched pair of cylindrical conduits. There may be little or no leak flow of air from the swivel in use.

Tie (noun): A structure designed to resist tension.

Vent: (noun): A structure that allows a flow of air from an interior of the mask, or conduit, to ambient air for clinically effective washout of exhaled gases. For example, a clinically effective washout may involve a flow rate of about 10 litres per minute to about 100 litres per minute, depending on the mask design and treatment pressure.

### 5.10.5 Shape of Structures

Products in accordance with the present technology may comprise one or more three-dimensional mechanical structures, for example a mask cushion or an impeller. The three-dimensional structures may be bounded by two-dimensional surfaces. These surfaces may be distinguished using a label to describe an associated surface orientation, location, function, or some other characteristic. For example a structure may comprise one or more of an anterior surface, a posterior surface, an interior surface and an exterior surface. In another example, a seal-forming structure may comprise a face-contacting (e.g. outer) surface, and a separate non-face-contacting (e.g. underside or inner) surface. In another example, a structure may comprise a first surface and a second surface.

To facilitate describing the shape of the three-dimensional structures and the surfaces, we first consider a cross-section through a surface of the structure at a point, p. See FIG. 3B to FIG. 3F, which illustrate examples of cross-sections at point p on a surface, and the resulting plane curves. FIGS. 3B to 3F also illustrate an outward normal vector at p. The outward normal vector at p points away from the surface. In some examples we describe the surface from the point of view of an imaginary small person standing upright on the surface.

### 5.10.5.1 Curvature in One Dimension

The curvature of a plane curve at p may be described as having a sign (e.g. positive, negative) and a magnitude (e.g. 1/radius of a circle that just touches the curve at p).

Positive curvature: If the curve at p turns towards the outward normal, the curvature at that point will be taken to be positive (if the imaginary small person leaves the point p they must walk uphill). See FIG. 3B (relatively large positive curvature compared to FIG. 3C) and FIG. 3C (relatively small positive curvature compared to FIG. 3B). Such curves are often referred to as concave.

Zero curvature: If the curve at p is a straight line, the curvature will be taken to be zero (if the imaginary small person leaves the point p, they can walk on a level, neither up nor down). See FIG. 3D.

Negative curvature: If the curve at p turns away from the outward normal, the curvature in that direction at that point will be taken to be negative (if the imaginary small person leaves the point p they must walk downhill). See FIG. 3E (relatively small negative curvature compared to FIG. 3F) and FIG. 3F (relatively large negative curvature compared to FIG. 3E). Such curves are often referred to as convex.

### 5.10.5.2 Curvature of Two Dimensional Surfaces

A description of the shape at a given point on a two-dimensional surface in accordance with the present technology may include multiple normal cross-sections. The multiple cross-sections may cut the surface in a plane that includes the outward normal (a “normal plane”), and each cross-section may be taken in a different direction. Each cross-section results in a plane curve with a corresponding curvature. The different curvatures at that point may have the same sign, or a different sign.

Each of the curvatures at that point has a magnitude, e.g. relatively small. The plane curves in FIGS. 3B to 3F could be examples of such multiple cross-sections at a particular point.

Principal curvatures and directions: The directions of the normal planes where the curvature of the curve takes its maximum and minimum values are called the principal directions. In the examples of FIG. 3B to FIG. 3F, the maximum curvature occurs in FIG. 3B, and the minimum occurs in FIG. 3F, hence FIG. 3B and FIG. 3F are cross sections in the principal directions. The principal curvatures at p are the curvatures in the principal directions.

Region of a surface: A connected set of points on a surface. The set of points in a region may have similar characteristics, e.g. curvatures or signs.

Saddle region: A region where at each point, the principal curvatures have opposite signs, that is, one is positive, and the other is negative (depending on the direction to which the imaginary person turns, they may walk uphill or downhill).

Dome region: A region where at each point the principal curvatures have the same sign, e.g. both positive (a “concave dome”) or both negative (a “convex dome”).

Cylindrical region: A region where one principal curvature is zero (or, for example, zero within manufacturing tolerances) and the other principal curvature is non-zero.

Planar region: A region of a surface where both of the principal curvatures are zero (or, for example, zero within manufacturing tolerances).

Edge of a surface: A boundary or limit of a surface or region.

Path: In certain forms of the present technology, ‘path’ will be taken to mean a path in the mathematical—topological sense, e.g. a continuous space curve from f(0) to f(1) on a surface. In certain forms of the present technology, a ‘path’ may be described as a route or course, including e.g. a set of points on a surface. (The path for the imaginary person is where they walk on the surface, and is analogous to a garden path).

Path length: In certain forms of the present technology, ‘path length’ will be taken to mean the distance along the surface from f(0) to f(1), that is, the distance along the path on the surface. There may be more than one path between two points on a surface and such paths may have different path lengths. (The path length for the imaginary person would be the distance they have to walk on the surface along the path).

Straight-line distance: The straight-line distance is the distance between two points on a surface, but without regard to the surface. On planar regions, there would be a path on the surface having the same path length as the straight-line distance between two points on the surface. On non-planar surfaces, there may be no paths having the same path length as the straight-line distance between two points. (For the imaginary person, the straight-line distance would correspond to the distance ‘as the crow flies’.)

5.10.5.3 Space curves Space curves: Unlike a plane curve, a space curve does not necessarily lie in any particular plane. A space curve may be closed, that is, having no endpoints. A space curve may be considered to be a one-dimensional piece of three-dimensional space. An imaginary person walking on a strand of the DNA helix walks along a space curve. A typical human left ear comprises a helix, which is a left-hand helix, see FIG. 3Q. A typical human right ear comprises a helix, which is a right-hand helix, see FIG. 3R. FIG. 3S shows a right-hand helix. The edge of a structure, e.g. the edge of a membrane or impeller, may follow a space curve. In general, a space curve may be described by a curvature and a torsion at each point on the space curve. Torsion is a measure of how the curve turns out of a plane. Torsion has a sign and a magnitude. The torsion at a point on a space curve may be characterised with reference to the Tangent, normal and binormal vectors at that point.

Tangent unit vector (or unit tangent vector): For each point on a curve, a vector at the point specifies a direction from that point, as well as a magnitude. A tangent unit vector is a unit vector pointing in the same direction as the curve at that point. If an imaginary person were flying along the curve and fell off her vehicle at a particular point, the direction of the tangent vector is the direction she would be travelling.

Unit normal vector: As the imaginary person moves along the curve, this tangent vector itself changes. The unit vector pointing in the same direction that the tangent vector is changing is called the unit principal normal vector. It is perpendicular to the tangent vector.

Binormal unit vector: The binormal unit vector is perpendicular to both the tangent vector and the principal normal vector. Its direction may be determined by a right-hand rule (see e.g. FIG. 3P), or alternatively by a left-hand rule (FIG. 3O).

Osculating plane: The plane containing the unit tangent vector and the unit principal normal vector. See FIGS. 3O and 3P.

Torsion of a space curve: The torsion at a point of a space curve is the magnitude of the rate of change of the binormal unit vector at that point. It measures how much the curve deviates from the osculating plane. A space curve which lies in a plane has zero torsion. A space curve which deviates a relatively small amount from the osculating plane will have a relatively small magnitude of torsion (e.g. a gently sloping helical path). A space curve which deviates a relatively large amount from the osculating plane will have a relatively large magnitude of torsion (e.g. a steeply sloping helical path). With reference to FIG. 3S, since T2>T1, the magnitude of the torsion near the top coils of the helix of FIG. 3S is greater than the magnitude of the torsion of the bottom coils of the helix of FIG. 3S

With reference to the right-hand rule of FIG. 3P, a space curve turning towards the direction of the right-hand binormal may be considered as having a right-hand positive torsion (e.g. a right-hand helix as shown in FIG. 3S). A space curve turning away from the direction of the right-hand binormal may be considered as having a right-hand negative torsion (e.g. a left-hand helix).

Equivalently, and with reference to a left-hand rule (see FIG. 3O), a space curve turning towards the direction of the left-hand binormal may be considered as having a left-hand positive torsion (e.g. a left-hand helix). Hence left-hand positive is equivalent to right-hand negative. See FIG. 3T.

### 5.10.5.4 Holes

A surface may have a one-dimensional hole, e.g. a hole bounded by a plane curve or by a space curve. Thin structures (e.g. a membrane) with a hole, may be described as having a one-dimensional hole. See for example the one dimensional hole in the surface of structure shown in FIG. 3I, bounded by a plane curve.

A structure may have a two-dimensional hole, e.g. a hole bounded by a surface. For example, an inflatable tyre has a two dimensional hole bounded by the interior surface of the tyre. In another example, a bladder with a cavity for air or gel could have a two-dimensional hole. See for example the cushion of FIG. 3L and the example cross-sections therethrough in FIG. 3M and FIG. 3N, with the interior surface bounding a two dimensional hole indicated. In a yet another example, a conduit may comprise a one-dimension hole (e.g. at its entrance or at its exit), and a two-dimension hole bounded by the inside surface of the conduit. See also the two dimensional hole through the structure shown in FIG. 3K, bounded by a surface as shown.

### 5.11 Other Remarks

A portion of the disclosure of this patent document contains material which is subject to copyright protection. The copyright owner has no objection to the facsimile reproduction by anyone of the patent document or the patent disclosure, as it appears in Patent Office patent files or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated herein by reference in their entirety to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

The terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular examples, it is to be understood that these examples are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative examples and that other arrangements may be devised without departing from the spirit and scope of the technology.

1.-69. (canceled)

70. A patient interface for delivering a flow of air to a patient for treatment of sleep disordered breathing, comprising:
a plenum chamber pressurisable to a therapeutic pressure of at least 4 cmH2O above ambient air pressure, said plenum chamber including a plenum chamber inlet port sized and structured to receive a flow of air at the therapeutic pressure for breathing by a patient; and
a seal-forming structure constructed and arranged to form a seal with a region of the patient's face surrounding an entrance to the patient's airways, said seal-forming structure having a hole therein such that the flow of air at said therapeutic pressure is delivered to at least an entrance to the patient's nares, the seal-forming structure constructed and arranged to maintain said therapeutic pressure in the plenum chamber throughout the patient's respiratory cycle in use; and
wherein the seal-forming structure includes a cushion having a plurality of interconnected struts forming a plurality of voids, and
wherein, in use, when the seal-forming structure is in engagement with the patient's face, the struts are configured to flex thereby altering the size, shape and/or orientation of the voids to allow the cushion to conform to the patient's face.

71. The patient interface of claim 70, wherein the struts are resilient.

72. The patient interface of claim 70,
wherein a characteristic of the cushion varies across the cushion such that in a first portion of the cushion the characteristic is different than in a second portion of the cushion, the first portion of the cushion having a level of flexibility that is different than the second portion of the cushion.

73. The patient interface of claim 72, wherein the characteristic of the cushion is 1) a thickness of the struts, 2) a density of the struts, 3) an orientation of the struts, 4) a spacing of the struts, 5) a size of the voids, 6) an orientation of the voids, and/or 7) a density of the voids.

74. The patient interface of claim 73, wherein the thickness of the struts in a first portion of the cushion is different than the thickness of the struts in a second portion of the cushion.

75. The patient interface of claim 73, wherein the size of the voids in the first portion of the cushion is different than the size of the voids in the second portion of the cushion.

76. The patient interface of claim 72, wherein the first portion of the cushion corresponds to a sensitive facial feature of the patient, and the second portion of the cushion does not correspond to a sensitive facial feature.

77. The patient interface of claim 76, wherein the sensitive facial feature is the patient's nasal ridge.

78. The patient interface of claim 72, wherein the first portion of the cushion has greater flexibility as compared to the second portion of the cushion.

79. The patient interface of claim 70, wherein the struts and voids form a lattice structure.

80. The patient interface of claim 70, wherein the cushion is not formed from a foam material.

81. The patient interface of claim 70, wherein the cushion is constructed from a foam material and has a plurality of macroscopic holes formed therein to form the voids.

82. The patient interface of claim 70, wherein the seal-forming structure is configured to seal to the patient's face at the patient's lip inferior, at the patient's cheeks, at the patient's lip superior, and at an inferior periphery of the patient's nose including the nasal alae and a pronasale region of the patient's nose.

83. The patient interface of claim 82, wherein the cushion comprises a lip inferior portion provided within a portion of the seal-forming structure configured to seal to the patient's face at the patient's lip inferior, the cushion comprises cheek portions provided within respective portions of the seal-forming structure configured to seal to the patient's face at the patient's cheeks, the cushion is stiffer in the cheek portions than in the lip inferior portion.

84. The patient interface of claim 70, wherein the struts and voids form a lattice structure, and
wherein the lattice structure comprises a plurality of unit cells, the lattice structure including one or more characteristics that vary between a patient-facing side of the cushion corresponding to a side of the seal-forming structure configured to contact the patient's face in use and a non-patient facing side of the cushion corresponding to a side of the seal-forming structure configured to face away from the patient's face in use.

85. The patient interface of claim 84, wherein the variation in the one or more characteristics of the lattice structure causes the cushion to be less stiff on the patient-facing side of the cushion than on the non-patient facing side of the cushion.

86. The patient interface of claim 70, wherein the patient interface comprises a positioning and stabilising structure configured to provide a force to hold the seal-forming structure in a therapeutically effective position on the patient's head when in use.

87. The patient interface of claim 86, wherein the positioning and stabilising structure comprises a pair of gas delivery tubes configured to provide the flow of air at therapeutic pressure to the plenum chamber and configured to provide a force to hold the seal-forming structure in sealing position, in use.

88. The patient interface of claim 70, wherein the cushion is configured to be removable from the patient interface.

89. The patient interface of claim 70, wherein the cushion forms a heat and moisture exchanger.
